(19) United States
(12) Patent Application Publication
(10) Pub. No.: US 2026/0186411 A1
(43) Pub. Date: Jul. 2, 2026

(54) PHOTORESIST COMPOSITIONS AND PATTERN FORMATION METHODS

(71) Applicant: DuPont Electronic Materials International, LLC, Marlborough, MA (US)

(72) Inventors: Li Cui, Westborough, MA (US); Vageesha Warnajith Liyana Gunawardana, Marlborough, MA (US); Emad Aqad, Northborough, MA (US); Jong Keun Park, Shrewsbury, MA (US)

(21) Appl. No.: 19/425,502

(22) Filed: Dec. 18, 2025

Related U.S. Application Data
(60) Provisional application No. 63/738,954, filed on Dec. 26, 2024.

(51) Int. Cl. G03F 7/004; C07C 381/12; G03F 7/038; G03F 7/039

(52) U.S. Cl. CPC G03F 7/0045; C07C 381/12; G03F 7/0382; G03F 7/0388; G03F 7/0392

(57) ABSTRACT

A photoresist composition including a first component; a second component; and a solvent, wherein the first component comprises a non-polymeric onium salt acid generator, and a cation of the non-polymeric onium salt generator comprises an acid labile group, wherein the second component comprises a hydroxyaryl group, and the second component does not comprise an acid labile group, and wherein the photoresist composition does not comprise a polymer that comprises an acid labile group.

### CROSS-REFERENCE TO RELATED APPLICATIONS

This application claims priority to and the benefit of U.S. Provisional Application Ser. No. 63/738,954, filed on Dec. 26, 2024, in the U.S. Patent and Trademark Office, the entire content of which is incorporated by reference herein.

### FIELD

The present invention relates to photoresist compositions including photoacid generator salts, and pattern formation methods. The invention finds particular applicability in lithographic applications in the semiconductor manufacturing industry.

### BACKGROUND

Photoresist materials are photosensitive compositions typically used for transferring an image to one or more underlying layers such as a metal, semiconductor or dielectric layer disposed on a semiconductor substrate. To increase the integration density of semiconductor devices and allow for the formation of structures having dimensions in the nanometer range, photoresists and photolithography processing tools having high-resolution capabilities have been and continue to be developed.

Chemically amplified resists (CAR) remain the mainstay technology in high resolution lithography. The past two decades of innovation within this platform has allowed the industry to print highly uniform features on the nanometer scale. This in turn has driven resist performance to meet the need for increasingly powerful semiconductors demanded by modern technological advancements.

There is a continuing need for photoresist compositions that improve multiple aspects of lithographic performance and for patterning methods using such photoresist compositions.

### SUMMARY

An aspect provides a photoresist composition including a first component; a second component; and a solvent, wherein the first component comprises a non-polymeric onium salt acid generator, and a cation of the non-polymeric onium salt generator comprises an acid labile group, wherein the second component comprises a hydroxyaryl group, and the second component does not comprise an acid labile group, and wherein the photoresist composition does not comprise a polymer that comprises an acid labile group.

Another aspect provides a method for patterning that includes applying a layer of a photoresist composition as described herein on a substrate to provide a photoresist composition layer; pattern-wise exposing the photoresist composition layer to activating radiation to provide an exposed photoresist composition layer; and developing the exposed photoresist composition layer to provide the pattern.

### DETAILED DESCRIPTION

Reference will now be made in detail to exemplary embodiments, examples of which are illustrated in the present description. In this regard, the present exemplary embodiments may have different forms and should not be construed as being limited to the descriptions set forth herein. Accordingly, the exemplary embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

As used herein, the terms “a,” “an,” and “the” do not denote a limitation of quantity and are to be construed to cover both the singular and the plural, unless otherwise indicated herein or clearly contradicted by context. “Or” means “and/or” unless clearly indicated otherwise. The modifier “about” used in connection with a quantity is inclusive of the stated value and has the meaning dictated by the context (e.g., includes the degree of error associated with measurement of the particular quantity). All ranges disclosed herein are inclusive of the endpoints, and the endpoints are independently combinable with each other. The suffix “(s)” is intended to include both the singular and the plural of the term that it modifies, thereby including at least one of that term. “Optional” or “optionally” means that the subsequently described event or circumstance can or cannot occur, and that the description includes instances where the event occurs and instances where it does not. The terms “first,” “second,” and the like, herein do not denote an order, quantity, or importance, but rather are used to distinguish one element from another. When an element is referred to as being “on” another element, it may be directly in contact with the other element or intervening elements may be present therebetween. In contrast, when an element is referred to as being “directly on” another element, there are no intervening elements present. It is to be understood that the described components, elements, limitations, and/or features of aspects may be combined in any suitable manner in the various aspects.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “actinic rays” or “radiation” means, for example, a bright line spectrum of a mercury lamp, far ultraviolet rays represented by an excimer laser, extreme ultraviolet rays (EUV light), X-rays, particle rays such as electron beams and ion beams, or the like. In addition, in the present invention, “light” means actinic rays or radiation. The krypton fluoride laser (KrF laser) is a particular type of excimer laser, which is sometimes referred to as an exciplex laser. “Excimer” is short for “excited dimer,” while “exciplex” is short for “excited complex.” An excimer laser uses a mixture of a noble gas (argon, krypton, or xenon) and a halogen gas (fluorine or chlorine), which under suitable conditions of electrical stimulation and high pressure, emits coherent stimulated radiation (laser light) in the ultraviolet range. Furthermore, “exposure” in the present specification includes, unless otherwise specified, not only exposure by a mercury lamp, far ultraviolet rays represented by an excimer laser, X-rays, extreme ultraviolet rays (EUV light), or the like, but also writing by particle rays such as electron beams and ion beams.

As used herein, the term “hydrocarbon” refers to an organic compound or group having at least one carbon atom and at least one hydrogen atom; “alkyl” refers to a straight or branched chain saturated hydrocarbon group having the specified number of carbon atoms and having a valence of one; “alkylene” refers to an alkyl group having a valence of two; “hydroxyalkyl” refers to an alkyl group substituted with at least one hydroxyl group (—OH); “alkoxy” refers to “alkyl-O—”; “carboxyl” and “carboxylic acid group” refer to a group having the formula “—C(O)—OH”; “cycloalkyl” refers to a monovalent group having one or more saturated rings in which all ring members are carbon; “cycloalkylene” refers to a cycloalkyl group having a valence of two; “alkenyl” refers to a straight or branched chain, monovalent hydrocarbon group having at least one carbon-carbon double bond; “alkenoxy” refers to “alkenyl-O—”; “alkenylene” refers to an alkenyl group having a valence of two; “cycloalkenyl” refers to a non-aromatic cyclic divalent hydrocarbon group having at least three carbon atoms, with at least one carbon-carbon double bond; “alkynyl” refers to a monovalent hydrocarbon group having at least one carbon-carbon triple bond; the term “aromatic group” refers to a monocyclic or polycyclic aromatic ring system that satisfies Huckel's Rule (4n+2 π electrons) and includes carbon atoms in the ring; the term “heteroaromatic group” refers to an aromatic group that includes one or more heteroatoms (e.g., 1-4 heteroatoms) selected from N, O, and S instead of a carbon atom in the ring; “aryl” refers to a monovalent monocyclic or polycyclic aromatic ring system where every ring member is carbon, and may include a group with an aromatic ring fused to at least one cycloalkyl or heterocycloalkyl ring; “arylene” refers to an aryl group having a valence of two; “alkylaryl” refers to an aryl group that has been substituted with an alkyl group; “arylalkyl” refers to an alkyl group that has been substituted with an aryl group; “aryloxy” refers to “aryl-O—”; and “arylthio” refers to “aryl-S—”.

The prefix “hetero” means that the compound or group includes at least one member that is a heteroatom (e.g., 1, 2, 3, or 4 or more heteroatom(s)) instead of a carbon atom, wherein the heteroatom(s) is each independently N, O, S, Si, or P; “heteroatom-containing group” refers to a substituent group that includes at least one heteroatom; “heteroalkyl” refers to an alkyl group having at least one heteroatom instead of carbon; “heterocycloalkyl” refers to a cycloalkyl group having 1-4 heteroatoms as ring members instead of carbon; “heterocycloalkylene” refers to a heterocycloalkyl group having a valence of two; “heteroaryl” refers to an aromatic 4-8 membered monocyclic, 8-12 membered bicyclic, or 11-14 membered tricyclic ring system having 1-4 heteroatoms (if monocyclic), 1-6 heteroatoms (if bicyclic), or 1-9 heteroatoms (if tricyclic) that are each independently selected from N, O, S, Si, or P (e.g., carbon atoms and 1-3, 1-6, or 1-9 heteroatoms of N, O, or S, if monocyclic, bicyclic, or tricyclic, respectively). Examples of heteroaryl groups include pyridyl, furyl (furyl or furanyl), imidazolyl, benzimidazolyl, pyrimidinyl, thiophenyl or thienyl, quinolinyl, indolyl, thiazolyl, and the like; and “heteroarylene” refers to a heteroaryl group having a valence of two.

The term “halogen” means a monovalent substituent that is fluorine (fluoro), chlorine (chloro), bromine (bromo), or iodine (iodo). The prefix “halo” means a group including one or more of a fluoro, chloro, bromo, or iodo substituent instead of a hydrogen atom. A combination of halo groups (e.g., bromo and fluoro), or only fluoro groups may be present. For example, the term “haloalkyl” refers to an alkyl group substituted with one or more halogens. As used herein, “substituted C₁₋₈ haloalkyl” refers to a C₁₋₈ alkyl group substituted with at least one halogen, and is further substituted with one or more other substituent groups that are not halogens. It is to be understood that substitution of a group with a halogen atom is not to be considered a heteroatom-containing group, because a halogen atom does not replace a carbon atom.

Each of the foregoing substituent groups optionally may be substituted unless expressly provided otherwise. The term “optionally substituted” refers to being substituted or unsubstituted. “Substituted” means that at least one hydrogen atom of the chemical structure or group is replaced with another terminal substituent group that is typically monovalent, provided that the designated atom's normal valence is not exceeded. When the substituent is oxo (i.e., O), then two geminal hydrogen atoms on the carbon atom are replaced with the terminal oxo group. It is further noted that the oxo group is bonded to carbon via a double bond to form a carbonyl (C═O), where the carbonyl group is represented herein as —C(O)—. Combinations of substituents or variables are permissible. Exemplary substituent groups that may be present on a “substituted” position include, but are not limited to, nitro (—NO₂), cyano (—CN), hydroxyl (—OH), oxo (O), amino (—NH₂), mono- or di-(C₁₋₆)alkylamino, alkanoyl (such as a C₂₋₆ alkanoyl group such as acyl), formyl (—C(O) H), carboxylic acid or an alkali metal or ammonium salt thereof; esters (including acrylates, methacrylates, and lactones) such as C₂₋₆ alkyl esters (—C(O)O-alkyl or —OC(O)-alkyl) and C₇₋₁₃ aryl esters (—C(O)O-aryl or —OC(O)-aryl); amido (—C(O)NR₂ wherein R is hydrogen or C₁₋₆ alkyl), carboxamido (—CH₂C(O)NR₂ wherein R is hydrogen or C₁₋₆ alkyl), halogen, thiol (—SH), C₁₋₆ alkylthio (—S-alkyl), thiocyano (—SCN), C₁₋₆ alkyl, C₂-6 alkenyl, C₂₋₆ alkynyl, C₁₋₆ haloalkyl, C₁₋₉ alkoxy, C₁₋₆ haloalkoxy, C₃₋₁₂ cycloalkyl, C₅₋₁₈ cycloalkenyl, C₂₋₁₈ heterocycloalkenyl, C₆₋₁₂ aryl having at least one aromatic ring (e.g., phenyl, biphenyl, naphthyl, or the like, each ring either substituted or unsubstituted aromatic), C₇₋₁₉ arylalkyl having 1 to 3 separate or fused rings and from 6 to 18 ring carbon atoms, arylalkoxy having 1 to 3 separate or fused rings and from 6 to 18 ring carbon atoms, C₇₋₁₂ alkylaryl, C₃₋₁₂ heterocycloalkyl, C₃-12 heteroaryl, C₁₋₆ alkyl sulfonyl (—S(O)₂-alkyl), C₆₋₁₂ arylsulfonyl (—S(O)₂-aryl), or tosyl (CH₃C₆H₄SO₂—). When a group is substituted, the indicated number of carbon atoms is the total number of carbon atoms in the group, excluding those of any substituents. For example, the group —CH₂CH₂CN is a cyano-substituted C₂ alkyl group.

As used herein, when a definition is not otherwise provided, a “divalent linking group” refers to a divalent group including one or more of —O—, —S—, —Te—, —Se—, —C(O)—, C(O)O—, —N(R′)—, —C(O)N(R′)—, —S(O)—, —S(O)₂—, —C(S)—, —C(Te)—, —C(Se)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein each R′ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. Typically, the divalent linking group includes one or more of —O—, —S—, —C(O)—, —N(R′)—, —S(O)—, —S(O)₂—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. More typically, the divalent linking group includes at least one of —O—, —C(O)—, —C(O)O—, —N(R′)—, —C(O)N(R′)—, substituted or unsubstituted C₁₋₁₀ alkylene, substituted or unsubstituted C₃₋₁₀ cycloalkylene, substituted or unsubstituted C₃₋₁₀ heterocycloalkylene, substituted or unsubstituted C₆₋₁₀ arylene, substituted or unsubstituted C₃₋₁₀ heteroarylene, or a combination thereof, wherein R is hydrogen, substituted or unsubstituted C₁₋₁₀ alkyl, substituted or unsubstituted C₁₋₁₀ heteroalkyl, substituted or unsubstituted C₆₋₁₀ aryl, or substituted or unsubstituted C₃₋₁₀ heteroaryl.

As used herein, an “acid-labile group” refers to a group in which a bond is cleaved by the action of an acid, optionally and typically with thermal treatment, resulting in formation of a polar group, such as a carboxylic acid or alcohol group. In some instances, the acid-labile group may be formed on a polymer, and optionally and typically with a moiety connected to the cleaved bond becoming disconnected from the polymer. In other systems, a non-polymeric compound may include an acid-labile group that may be cleaved by the action of an acid, resulting in formation of a polar group, such as a carboxylic acid or alcohol group on a cleaved portion of the non-polymeric compound. Such acid is typically a photo-generated acid with bond cleavage occurring during post-exposure baking (PEB); however, embodiments are not limited thereto, and, for example, such acid may be thermally generated. Suitable acid-labile groups include, for example: tertiary alkyl ester groups, secondary or tertiary ester groups having aryl groups, secondary or tertiary ester groups having a combination of alkyl and aryl groups, tertiary alkoxy groups, acetal groups, or ketal groups. Acid-labile groups are also commonly referred to in the art as “acid-cleavable groups,” “acid-cleavable protecting groups,” “acid-labile protecting groups,” “acid-leaving groups,” “acid-decomposable groups,” and “acid-sensitive groups.”

Improving feature uniformity remains one of the primary challenges of photolithography. Given that CAR materials are comprised of multiple components-typically, polymer, photoacid generator (PAG), photo destroyable quencher (PDQ), and other additives-differences in affinities that each of them has with the surrounding matrix can result in non-unform distribution within the film. Additionally, the dispersity associated with the polymer itself can result in inhomogeneities, since the differences in the chain length and compositions can have drastically different physical properties. Therefore, film homogeneity remains a key attribute for improving feature uniformity in high resolution lithography.

The present inventors have discovered a molecular photoresist platform for providing homogeneous photoresists, which may eliminate the need for a polymer having separate acid switchable functionality by incorporating this functionality into the photoacid generator (PAG) itself. In the molecular photoresists described herein, the acid generating component and the solubility switching component are part of the same molecule. By decreasing the number of components added, the inventors have provided a photoresist composition that is inherently more homogeneous as compared to photoresist compositions that include an acid switchable polymer.

Provided is a photoresist composition that includes a first component; a second component; and a solvent, wherein the first component includes a non-polymeric onium salt acid generator, and a cation of the non-polymeric onium salt generator includes an acid labile group, wherein the second component includes a hydroxyaryl group, and the second component does not include an acid labile group, and wherein the photoresist composition does not include a polymer that includes an acid labile group.

The first component includes a non-polymeric onium salt acid generator, and a cation of the non-polymeric onium salt generator includes an acid labile group. In some embodiments, the first component may be a salt that includes a cation of Formula (1) or (2):

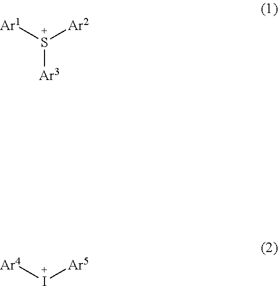

In Formulae (1) and (2), Ar¹ to Ar⁵ are each independently substituted or unsubstituted C₆₋₃₀ aryl, or substituted or unsubstituted C₃₋₃₀ heteroaryl. The C₆₋₃₀ aryl and the C₃₋₃₀ heteroaryl groups may each be monocyclic or polycyclic. It is to be understood that when the “monocyclic or polycyclic C₃₋₃₀ aryl group” is polycyclic, the number of carbon atoms is sufficient for the group to be chemically feasible. For example, the “monocyclic or polycyclic C₆₋₃₀ aryl” may refer to “a monocyclic C₆ aryl group or a polycyclic C₁₀₋₃₀ aryl group”. Similarly, when the “monocyclic or polycyclic C₃₋₃₀ heteroaryl” is polycyclic, the number of carbon atoms is sufficient for the group to be chemically feasible. For example, the “monocyclic or polycyclic C₃₋₃₀ heteroaryl” may refer to “a monocyclic C₃₋₆ heteroaryl or a polycyclic C₅₋₃₀ heteroaryl”. Exemplary groups for Ar¹ to Ar⁵ include, but are not limited to, benzene, naphthalene, anthracene, phenanthrene, pyrene, coronene, triphenylene, chrysene, phenalene, benz[a]anthracene, dibenz[a,h]anthracene, or benzo[a]pyrene, each of which may be substituted or unsubstituted. Typically, Ar¹ to Ar⁵ are benzene, which may be substituted or unsubstituted.

When the C₆₋₃₀ aryl and the C₃₋₃₀ heteroaryl groups are polycyclic, the ring or ring groups can be fused (such as naphthyl or the like), directly linked (such as biaryls, biphenyl, or the like), and/or bridged by a heteroatom (such as triphenylamino or diphenylene ether).

In Formula (1), each of Ar¹ to Ar³ may be either separate or connected to another group Ar¹ to Ar³ via a single bond or a divalent linking group to form a ring. For example, two or more of Ar¹ to Ar³ may be connected to each other via a single bond or a divalent linking group to form a ring. In some embodiments, Ar¹ and Ar² may be connected to each other via a single bond.

In Formula (2), each of Ar⁴ to Ar⁵ may be either separate or connected to another group Ar⁴ to Ar⁵ via a single bond or a divalent linking group to form a ring. For example, Ar⁴ and Ar⁵ may be connected to each other via a single bond or a divalent linking group to form a ring.

In Formulae (1) and (2), at least one of Ar¹ to Ar³ in Formula (1) and at least one of Ar⁴ to Ar⁵ in Formula (2) includes an acid labile group. Exemplary acid labile groups include tertiary alkyl ester groups, secondary or tertiary aryl ester groups, secondary or tertiary ester groups having a combination of alkyl and aryl groups, tertiary alkoxy groups, acetal groups, or ketal groups.

The acid labile group may be bonded directly to any of Ar¹ to Ar⁵, or may be bonded to any of Ar¹ to Ar⁵ through one or more divalent linking groups. Exemplary divalent linking groups may include, but are not limited to, —O—, —S—, —C(O)—, —C(O)O—, —N(R′)—, —C(O)N(R′)—, —S(O)—, —S(O)₂—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In some embodiments, each of Ar¹ to Ar⁵ optionally may include as part of its structure one or more groups chosen from —O—, —C(O)—, —C(O)—O—, —C₁₋₁₂ hydrocarbylene-, —O—(C₁₋₁₂ hydrocarbylene)-, —C(O)—O—(C₁₋₁₂ hydrocarbylene)-, and —C(O)—O—(C₁₋₁₂ hydrocarbylene)-O—.

Exemplary cations of Formula (1) and (2) include the following:

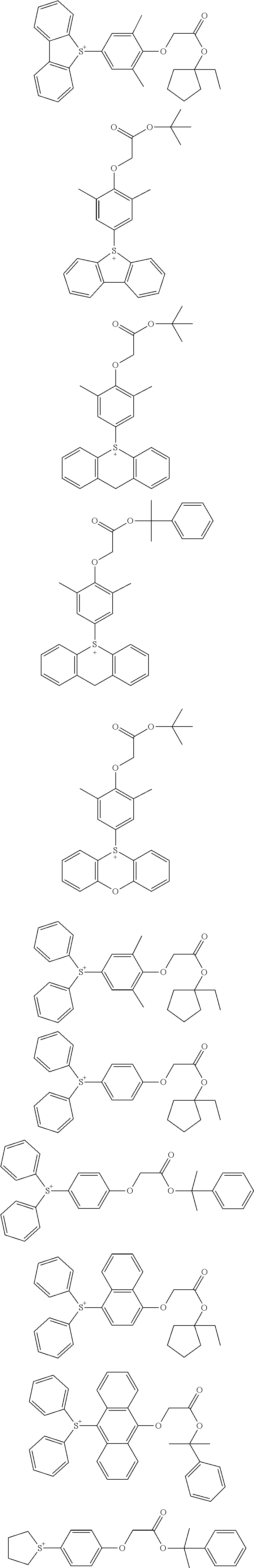

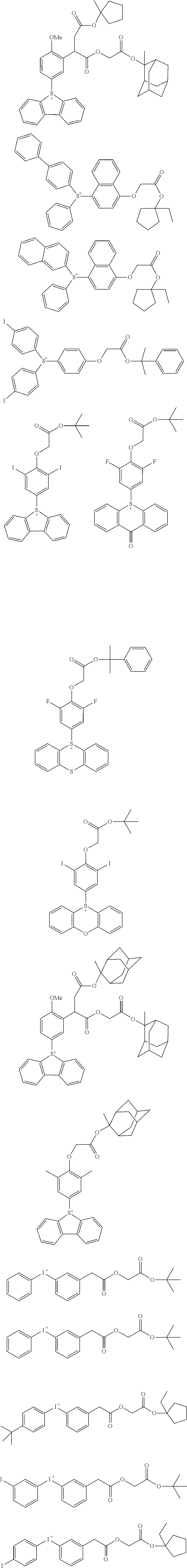

In some embodiments, in Formulae (1) and (2), at least one of Ar¹ to Ar³ in Formula (1) and at least one of Ar⁴ to Ar⁵ in Formula (2) is of Formula (A):

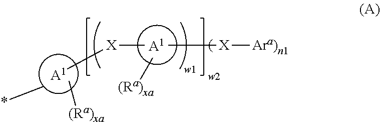

In Formula (A), each ring A¹ is independently C₆-30 aryl or C₃₋₃₀ heteroaryl. The C₆₋₃₀ aryl and the C₃₋₃₀ heteroaryl groups may each be monocyclic or polycyclic, as described above for Ar¹ to Ar⁵. Exemplary groups for ring A¹ include, but are not limited to, benzene, naphthalene, anthracene, phenanthrene, pyrene, coronene, triphenylene, chrysene, phenalene, benz[a]anthracene, dibenz[a,h]anthracene, or benzo[a]pyrene, each of which may be substituted or unsubstituted. Typically, ring A¹ is benzene.

In Formula (A), each R^{a} is independently a non-hydrogen substituent. For example, each R^{a} may be independently deuterium, halogen, substituted or unsubstituted C₁₋₃₀ alkyl, substituted or unsubstituted C₃₋₃₀ cycloalkyl, substituted or unsubstituted C₃₋₃₀ cycloalkene, substituted or unsubstituted C₃₋₃₀ heterocycloalkyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₆₋₃₀ aryloxy, substituted or unsubstituted C₃₋₃₀ heteroaryl, substituted or unsubstituted C₄₋₃₀ alkylheteroaryl, substituted or unsubstituted C₄₋₃₀ heteroarylalkyl, or substituted or unsubstituted C₃₋₃₀ heteroaryloxy.

In Formula (A), each R^{a} optionally further comprises one or more divalent linking groups as part of its structure. Each of the one or more divalent linking groups may be substituted or unsubstituted. Exemplary divalent linking groups may be selected from —O—, —C(O)—, —C(O)O—, —S—, —S(O)—, —S(O)₂—, —N(R′)—, —C(O)N(R′)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ may be hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formula (A), each X is independently a single bond or one or more of substituted or unsubstituted C₁₋₁₀ alkylene, substituted or unsubstituted C₃₋₁₀ cycloalkylene, substituted or unsubstituted C₃₋₁₀ heterocycloalkylene, substituted or unsubstituted C₆₋₁₀ arylene, substituted or unsubstituted C₃₋₁₀ heteroarylene, O, S, S(O), C(O), C(O)O, OC(O), C(O)N(R′), or NR′, wherein R′ is hydrogen, deuterium, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₃₀ aryl, substituted or unsubstituted C₃₋₃₀ heteroaryl, or Ar^{a}. In one or more embodiments, each X is independently O, S, or NR′, wherein R′ is hydrogen, deuterium, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₃₀ aryl, substituted or unsubstituted C₃₋₃₀ heteroaryl, or Ar^{a}. It is to be understood that when R′ is Ar^{a}, then this represents a second or additional group Ar^{a}, which is in addition to the group Ar^{a} depicted in Formula (A). Typically, each X is independently O or S.

In Formula (A), n1 is an integer from 1 to 4. Typically, n1 is 1 or 2.

In Formula (A), each xa is independently an integer from 0 to 10. For example, each xa may independently be 0, 1, or 2.

In Formula (A), w1 is an integer from 1 to 4.

In Formula (A), w2 is an integer from 0 to 4. Typically, w2 is 0.

In Formula (A), * indicates a binding site to an adjacent atom.

In Formula (A), each Ar^{a} is independently a group of Formula (B) or (C):

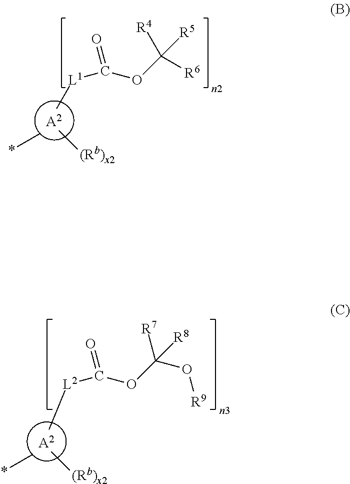

In Formulae (B) and (C), ring A² is C₆₋₃₀ aryl or C₃₋₃₀ heteroaryl. The C₆₋₃₀ aryl and the C₃₋₃₀ heteroaryl groups may each be monocyclic or polycyclic, as described above for Ar¹ to Ar⁵. Exemplary groups for ring A² include, but are not limited to, benzene, naphthalene, anthracene, phenanthrene, pyrene, coronene, triphenylene, chrysene, phenalene, benz[a]anthracene, dibenz[a,h]anthracene, or benzo[a]pyrene, each of which may be substituted or unsubstituted. Typically, ring A² is benzene.

In Formulae (B) and (C), L¹ and L² are each independently a single bond or a divalent linking group selected from substituted or unsubstituted C₁₋₁₀ alkylene, substituted or unsubstituted C₃₋₂₀ cycloalkylene, substituted or unsubstituted C₆₋₃₀ arylene group, substituted or unsubstituted C₃₋₃₀ heteroarylene, or a combination thereof. Typically, L¹ and L² are each independently a single bond.

In Formulae (B) and (C), each R^{b} is independently a non-hydrogen substituent. For example, each R^{b} may be independently deuterium, halogen, substituted or unsubstituted C₁₋₃₀ alkyl, substituted or unsubstituted C₃₋₃₀ cycloalkyl, substituted or unsubstituted C₃₋₃₀ cycloalkene, substituted or unsubstituted C₃₋₃₀ heterocycloalkyl, substituted or unsubstituted C₆-30 aryl, substituted or unsubstituted C₇₋₃₀ arylalkyl, substituted or unsubstituted C₇₋₃₀ alkylaryl, substituted or unsubstituted C₆₋₃₀ aryloxy, substituted or unsubstituted C₃₋₃₀ heteroaryl, substituted or unsubstituted C₄₋₃₀ alkylheteroaryl, substituted or unsubstituted C₄₋₃₀ heteroarylalkyl, or substituted or unsubstituted C₃₋₃₀ heteroaryloxy.

In Formulae (B) and (C), each R^{b} optionally further comprises one or more divalent linking groups as part of its structure. Each of the one or more divalent linking groups may be substituted or unsubstituted. Exemplary divalent linking groups may be selected from —O—, —C(O)—, —C(O)O—, —S—, —S(O)—, —S(O)₂—, —N(R′)—, —C(O)N(R′)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ may be hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In some embodiments, one or more R^{b} may each independently further include a lactone-containing group, hydroxyaryl group a base-solubilizing group, or the like, or a combination thereof as part of their structure.

In Formulae (B) and (C), x2 is independently an integer from 0 to 10. For example, each x2 may independently be 0, 1, or 2.

In Formula (B), R⁴ to R⁶ are each independently hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₂₋₂₀ alkenyl, substituted or unsubstituted C₃₋₂₀ cycloalkenyl, substituted or unsubstituted C₂-C₂₀ alkynyl, substituted or unsubstituted C₃₋₂₀ heterocycloalkenyl, substituted or unsubstituted C₆₋₂₀ aryl, or substituted or unsubstituted C₃₋₂₀ heteroaryl, provided that no more than one of R⁴ to R⁶ may be hydrogen and provided that if one of R⁴ to R⁶ is hydrogen, then at least one of the others from R⁴ to R⁶ is substituted or unsubstituted C₆₋₂₀ aryl or substituted or unsubstituted C₃₋₂₀ heteroaryl. Preferably, R⁴ to R⁶ are each independently substituted or unsubstituted C₁₋₆ alkyl or substituted or unsubstituted C₃₋₁₀ cycloalkyl. Each of R⁴ to R⁶ may optionally further comprise a divalent linking group as part of their structure.

For example, any one or more of R⁴ to R⁶ may be independently a group of the formula-CH₂C(O)CH₍₃₋ₙ₎Yₙ, or —CH₂C(O)OCH₍₃₋ₙ₎Yₙ, where each Y is independently substituted or unsubstituted C₃₋₁₀ heterocycloalkyl and n is 1 or 2. For example, each Y may be independently substituted or unsubstituted C₃₋₁₀ heterocycloalkyl including a group of the formula-O(C^{a1})(C^{a2})O—, wherein C^{a1} and C^{a2} are each independently hydrogen or substituted or unsubstituted alkyl, and where C^{a1} and C^{a2} together optionally form a ring.

In Formula (B), any two of R⁴ to R⁶ together optionally form a ring, which may further comprise a divalent linking group as part of its structure, and wherein the ring may be substituted or unsubstituted.

In Formula (C), R⁷ and R⁸ are each independently hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, or substituted or unsubstituted C₃₋₂₀ heteroaryl. Preferably, R⁷ and R⁸ each independently may be hydrogen, substituted or unsubstituted C₁₋₂₀ alkyl, substituted or unsubstituted C₃₋₂₀ cycloalkyl, or substituted or unsubstituted C₃₋₂₀ heterocycloalkyl. Each of R⁷ and R⁸ may optionally further comprise a divalent linking group as part of their structure.

In Formula (C), R⁹ is substituted or unsubstituted C₁₋₂₀ alkyl, substituted or unsubstituted C₃₋₂₀ cycloalkyl, or substituted or unsubstituted C₃₋₂₀ heterocycloalkyl.

In Formula (C), any two of R⁷ to R⁹ together optionally form a ring, which may further comprise a divalent linking group as part of its structure, wherein the ring group may be substituted or unsubstituted.

In some aspects, each of R⁴ to R⁹ optionally may include as part of their structure one or more divalent linking groups selected from —O—, —C(O)—, —C(O)—O—, —S—, —S(O)₂—, and N(R′)—S(O)₂—, wherein R′ may be hydrogen, substituted or unsubstituted C₁₋₂₀ alkyl, substituted or unsubstituted C₃₋₂₀ cycloalkyl, or substituted or unsubstituted C₃₋₂₀ heterocycloalkyl.

In Formula (B), n2 is an integer from 1 to 4. Typically, n2 is 1 or 2.

In Formula (C), n3 is an integer from 1 to 4. Typically, n3 is 1 or 2.

In Formulae (B) and (C), * indicates a binding site to an adjacent atom.

Non-limiting examples of the cations that include a group of Formula (A) include the following:

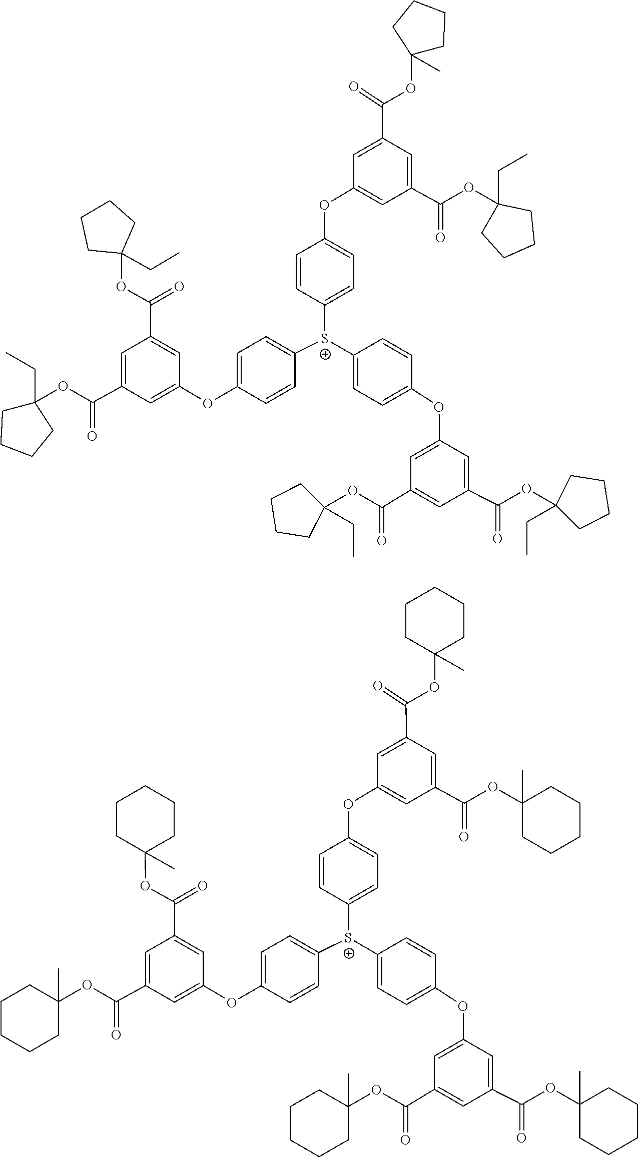 [SMILES: CCC1(OC(=O)c2cc(Oc3ccc([S+](c4ccc(Oc5cc(C(=O)OC6(CC)CCCC6)cc(C(=O)OC6(CC)CCCC6)c5)cc4)c4ccc(Oc5cc(C(=O)OC6(CC)CCCC6)cc(C(=O)OC6(CC)CCCC6)c5)cc4)cc3)cc(C(=O)OC3(C)CCCC3)c2)CCCC1 ; CC1(OC(=O)c2cc(Oc3ccc([S+](c4ccc(Oc5cc(C(=O)OC6(C)CCCCC6)cc(C(=O)OC6(C)CCCCC6)c5)cc4)c4ccc(Oc5cc(C(=O)OC6(C)CCCCC6)cc(C(=O)OC6(C)CCCCC6)c5)cc4)cc3)cc(C(=O)OC3(C)CCCCC3)c2)CCCCC1]

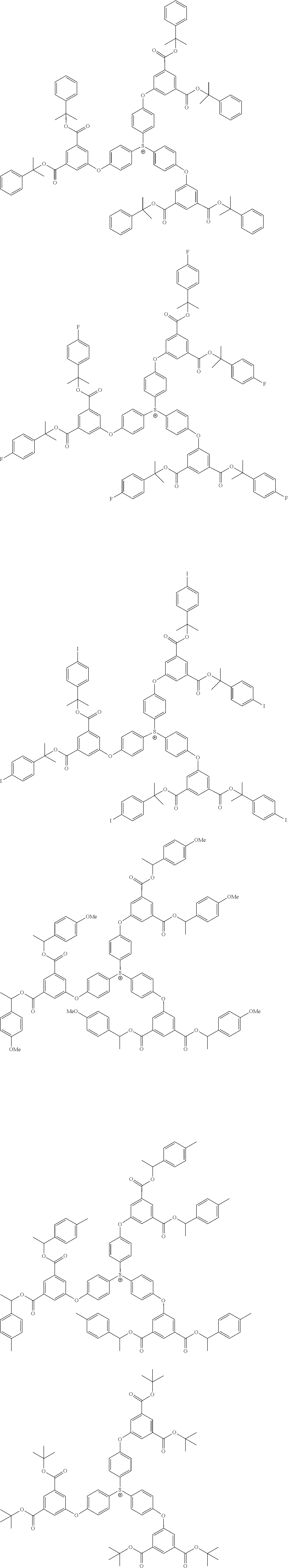

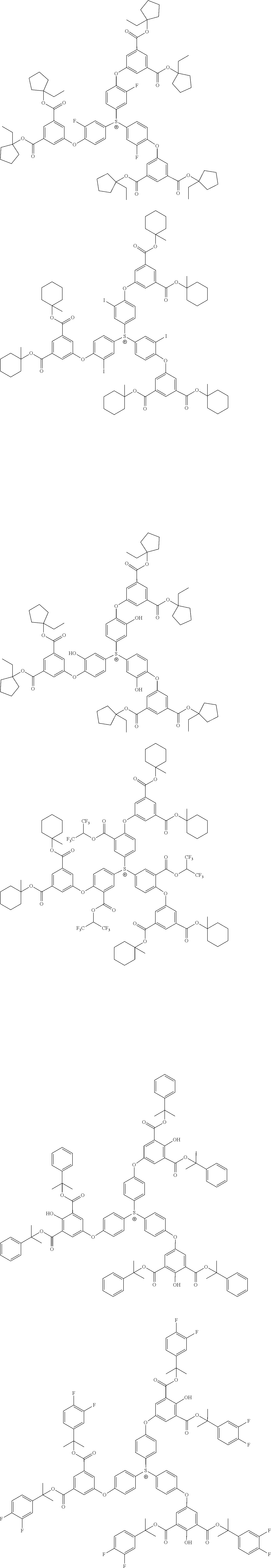

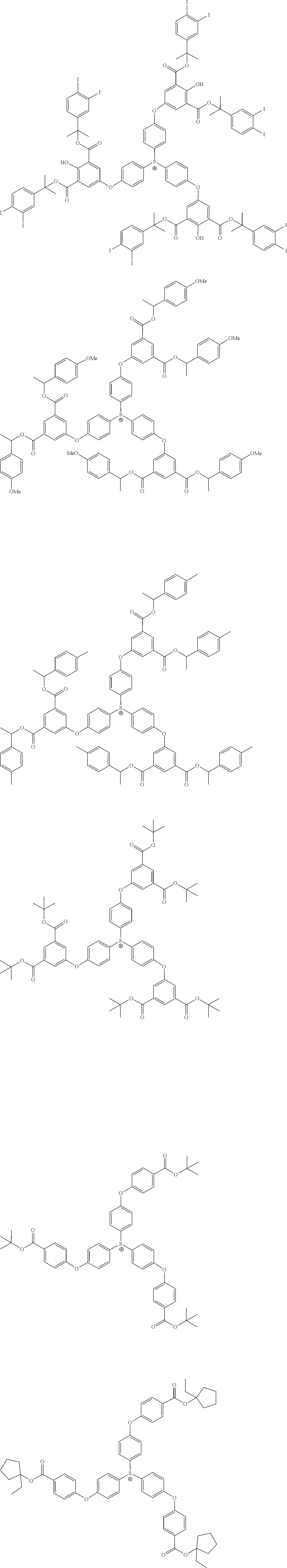

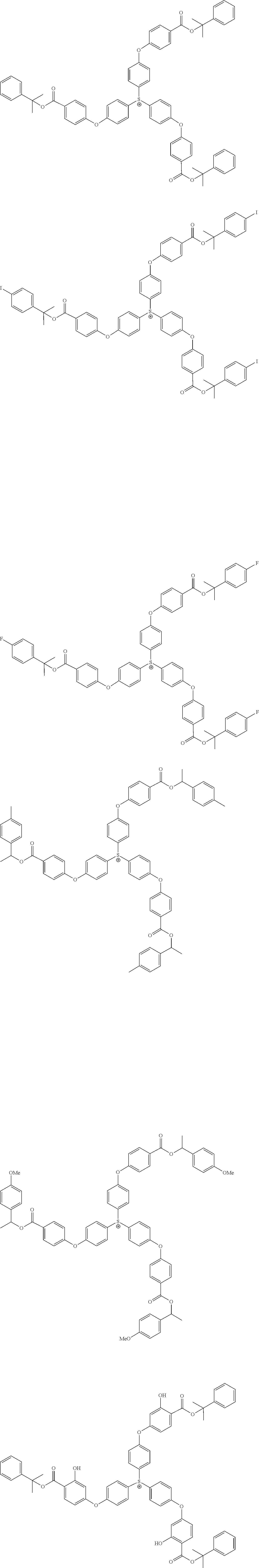

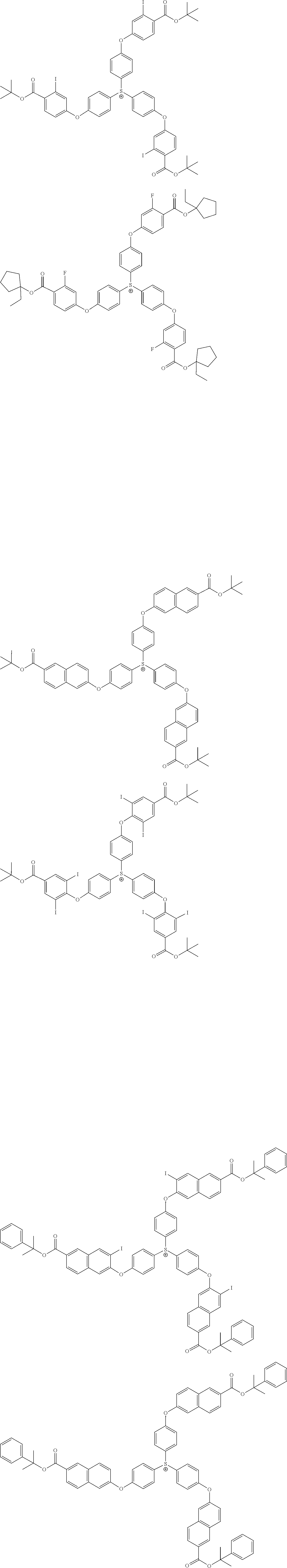

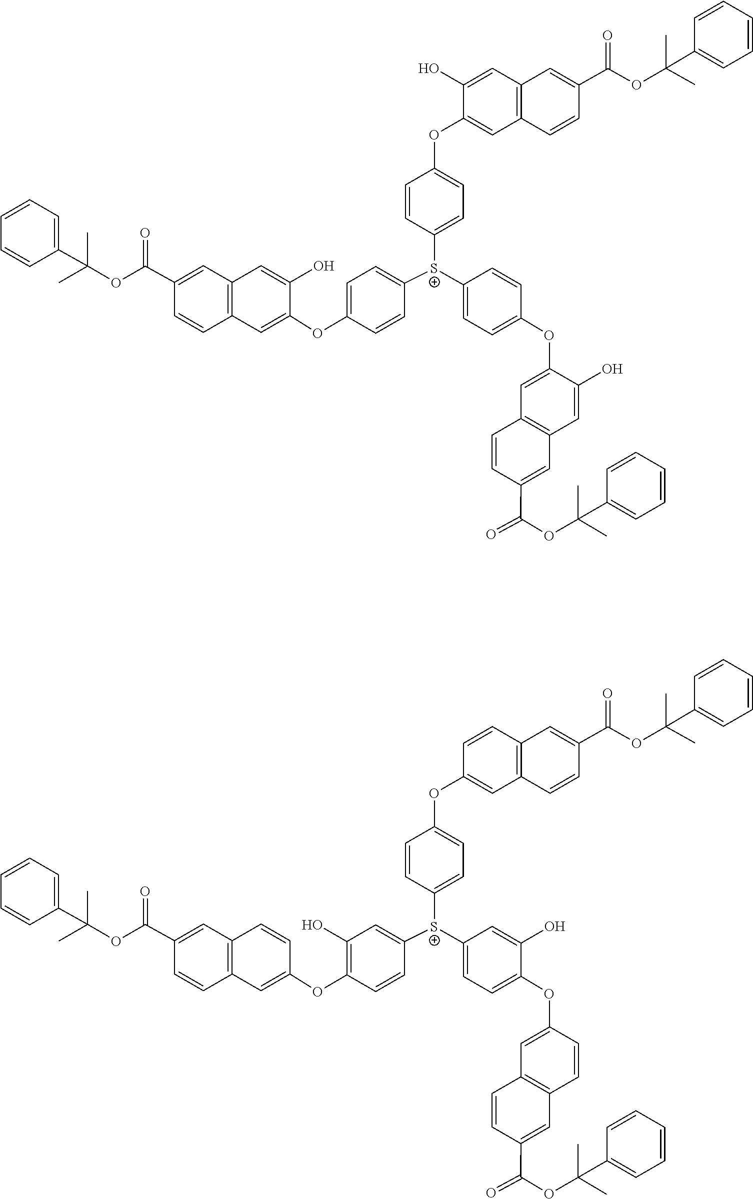 [SMILES: CC(C)(OC(=O)c1ccc2cc(Oc3ccc([S+](c4ccc(Oc5cc6ccc(C(=O)OC(C)(C)c7ccccc7)cc6cc5O)cc4)c4ccc(Oc5cc6ccc(C(=O)OC(C)(C)c7ccccc7)cc6cc5O)cc4)cc3)c(O)cc2c1)c1ccccc1 ; CC(C)(OC(=O)c1ccc2cc(Oc3ccc([S+](c4ccc(Oc5ccc6cc(C(=O)OC(C)(C)c7ccccc7)ccc6c5)c(O)c4)c4ccc(Oc5ccc6cc(C(=O)OC(C)(C)c7ccccc7)ccc6c5)c(O)c4)cc3)ccc2c1)c1ccccc1]

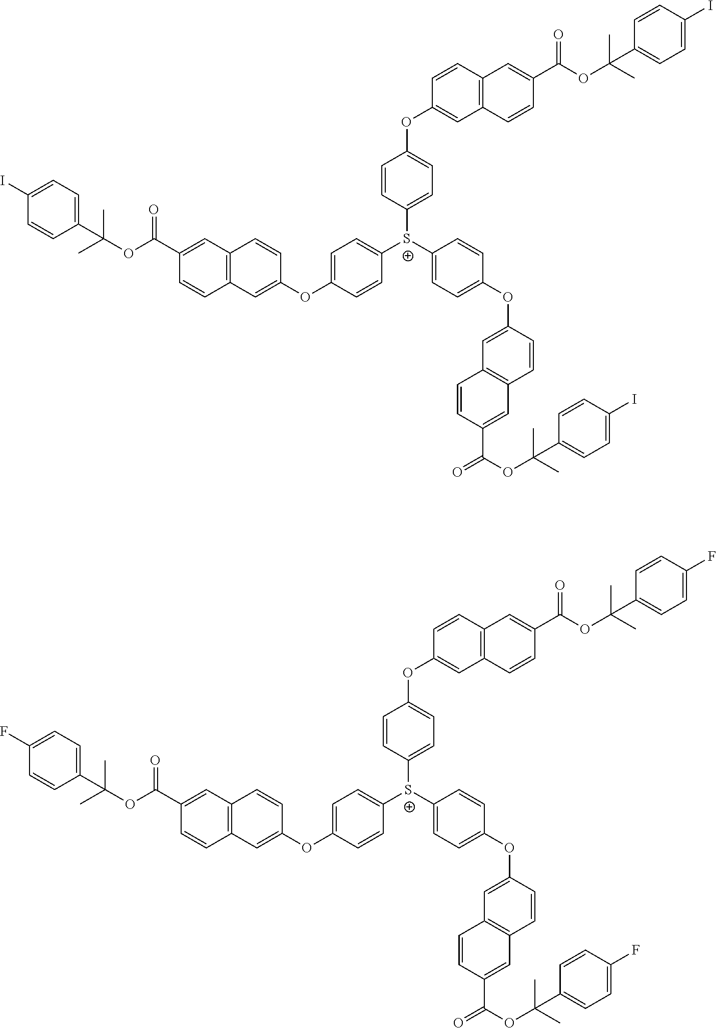 [SMILES: CC(C)(OC(=O)c1ccc2cc(Oc3ccc([S+](c4ccc(Oc5ccc6cc(C(=O)OC(C)(C)c7ccc(I)cc7)ccc6c5)cc4)c4ccc(Oc5ccc6cc(C(=O)OC(C)(C)c7ccc(I)cc7)ccc6c5)cc4)cc3)ccc2c1)c1ccc(I)cc1 ; CC(C)(OC(=O)c1ccc2cc(Oc3ccc([S+](c4ccc(Oc5ccc6cc(C(=O)OC(C)(C)c7ccc(F)cc7)ccc6c5)cc4)c4ccc(Oc5ccc6cc(C(=O)OC(C)(C)c7ccc(F)cc7)ccc6c5)cc4)cc3)ccc2c1)c1ccc(F)cc1]

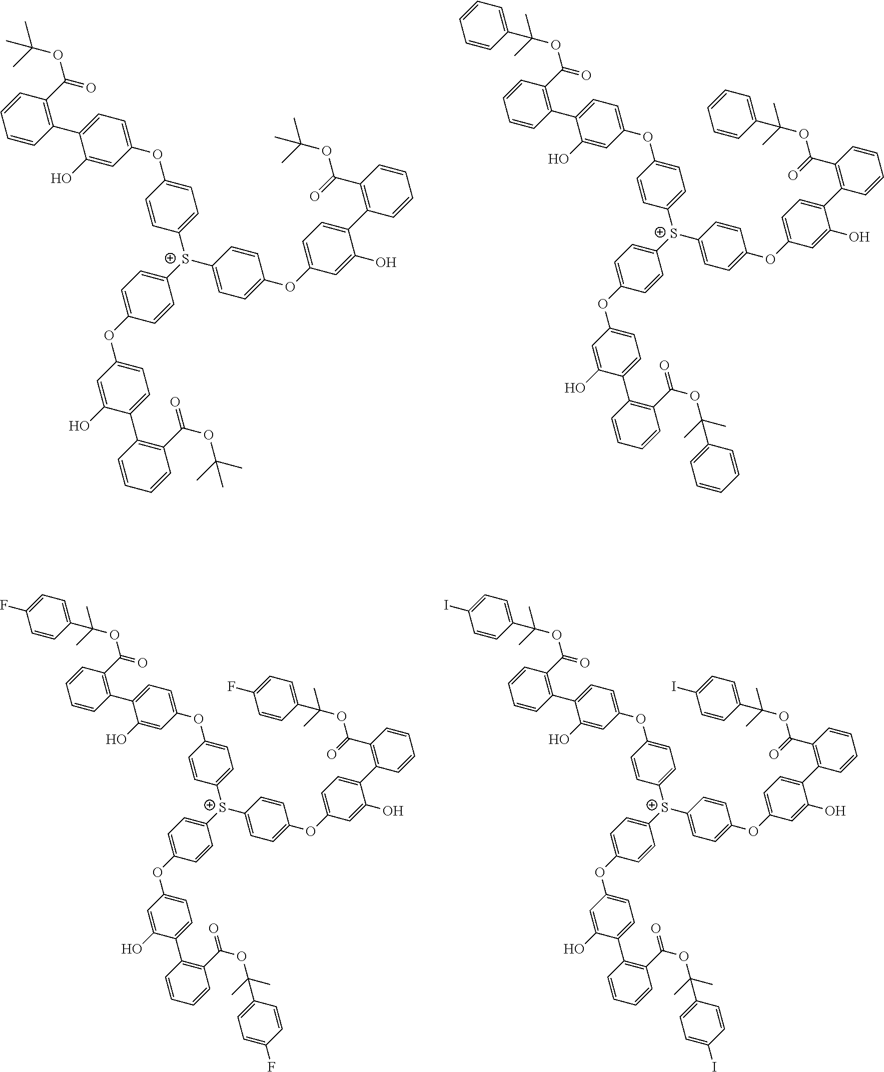

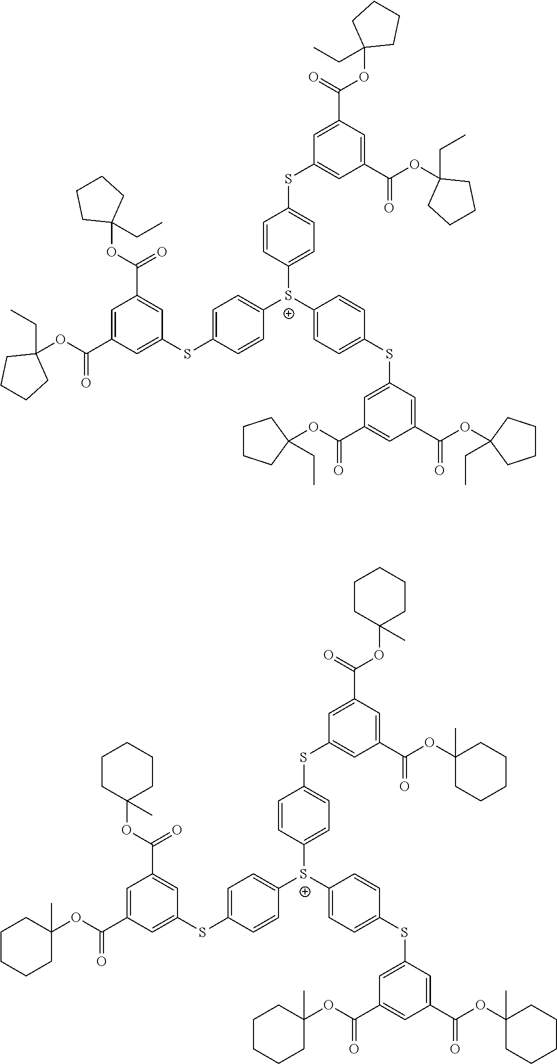 [SMILES: CCC1(OC(=O)c2cc(Sc3ccc([S+](c4ccc(Sc5cc(C(=O)OC6(CC)CCCC6)cc(C(=O)OC6(CC)CCCC6)c5)cc4)c4ccc(Sc5cc(C(=O)OC6(CC)CCCC6)cc(C(=O)OC6(CC)CCCC6)c5)cc4)cc3)cc(C(=O)OC3(CC)CCCC3)c2)CCCC1 ; CC1(OC(=O)c2cc(Sc3ccc([S+](c4ccc(Sc5cc(C(=O)OC6(C)CCCCC6)cc(C(=O)OC6(C)CCCCC6)c5)cc4)c4ccc(Sc5cc(C(=O)OC6(C)CCCCC6)cc(C(=O)OC6(C)CCCCC6)c5)cc4)cc3)cc(C(=O)OC3(C)CCCCC3)c2)CCCCC1]

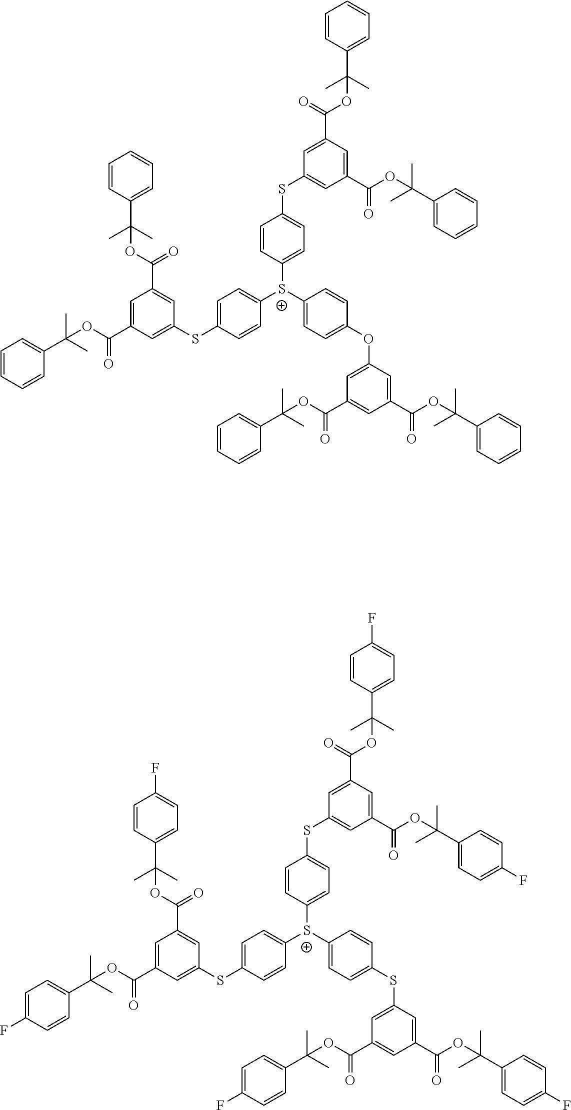 [SMILES: CC(C)(OC(=O)c1cc(Oc2ccc([S+](c3ccc(Sc4cc(C(=O)OC(C)(C)c5ccccc5)cc(C(=O)OC(C)(C)c5ccccc5)c4)cc3)c3ccc(Sc4cc(C(=O)OC(C)(C)c5ccccc5)cc(C(=O)OC(C)(C)c5ccccc5)c4)cc3)cc2)cc(C(=O)OC(C)(C)c2ccccc2)c1)c1ccccc1 ; CC(C)(OC(=O)c1cc(Sc2ccc([S+](c3ccc(Sc4cc(C(=O)OC(C)(C)c5ccc(F)cc5)cc(C(=O)OC(C)(C)c5ccc(F)cc5)c4)cc3)c3ccc(Sc4cc(C(=O)OC(C)(C)c5ccc(F)cc5)cc(C(=O)OC(C)(C)c5ccc(F)cc5)c4)cc3)cc2)cc(C(=O)OC(C)(C)c2ccc(F)cc2)c1)c1ccc(F)cc1]

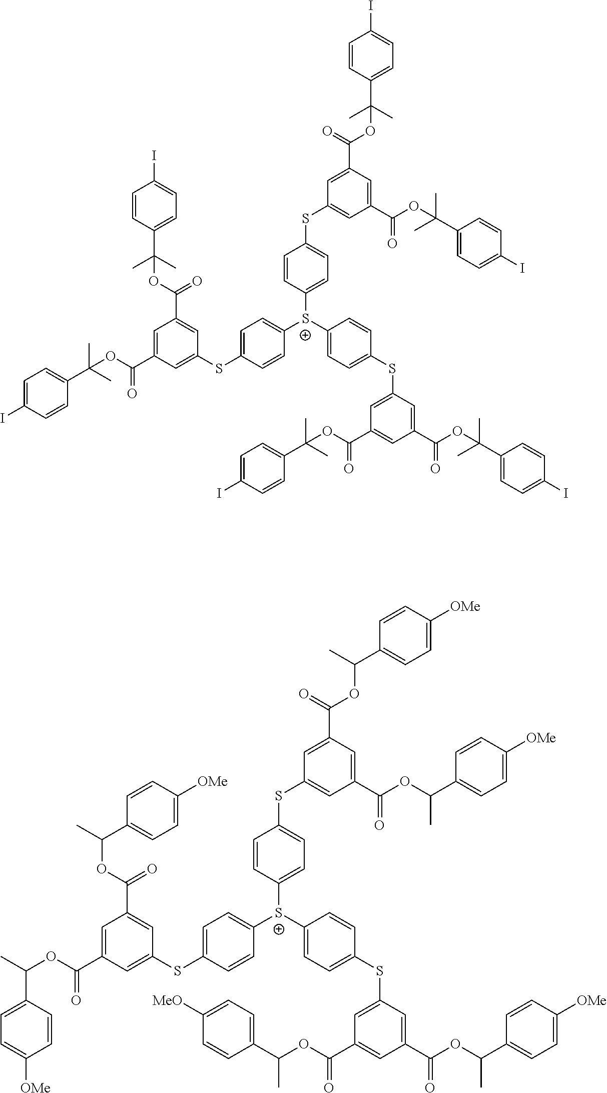 [SMILES: CC(C)(OC(=O)c1cc(Sc2ccc([S+](c3ccc(Sc4cc(C(=O)OC(C)(C)c5ccc(I)cc5)cc(C(=O)OC(C)(C)c5ccc(I)cc5)c4)cc3)c3ccc(Sc4cc(C(=O)OC(C)(C)c5ccc(I)cc5)cc(C(=O)OC(C)(C)c5ccc(I)cc5)c4)cc3)cc2)cc(C(=O)OC(C)(C)c2ccc(I)cc2)c1)c1ccc(I)cc1 ; COc1ccc(C(C)OC(=O)c2cc(Sc3ccc([S+](c4ccc(Sc5cc(C(=O)OC(C)c6ccc(OC)cc6)cc(C(=O)OC(C)c6ccc(OC)cc6)c5)cc4)c4ccc(Sc5cc(C(=O)OC(C)c6ccc(OC)cc6)cc(C(=O)OC(C)c6ccc(OC)cc6)c5)cc4)cc3)cc(C(=O)OC(C)c3ccc(OC)cc3)c2)cc1]

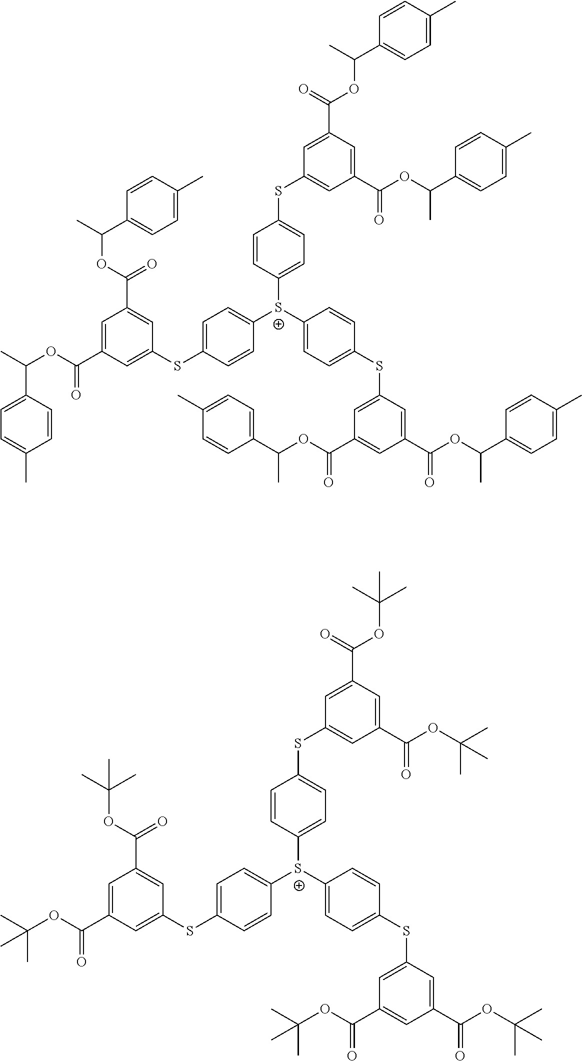

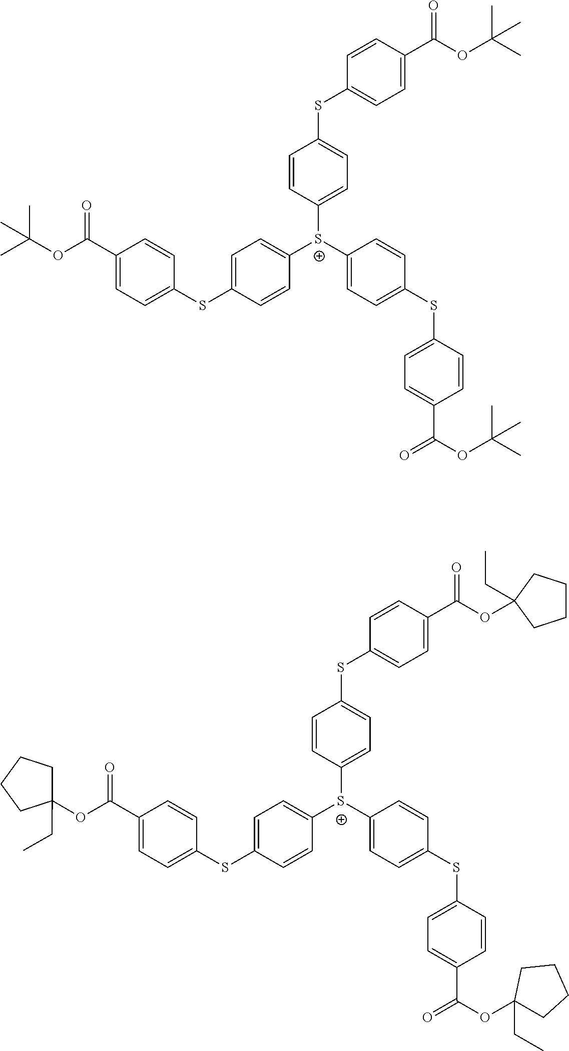 [SMILES: CC(C)(C)OC(=O)c1ccc(Sc2ccc([S+](c3ccc(Sc4ccc(C(=O)OC(C)(C)C)cc4)cc3)c3ccc(Sc4ccc(C(=O)OC(C)(C)C)cc4)cc3)cc2)cc1 ; CCC1(OC(=O)c2ccc(Sc3ccc([S+](c4ccc(Sc5ccc(C(=O)OC6(CC)CCCC6)cc5)cc4)c4ccc(Sc5ccc(C(=O)OC6(CC)CCCC6)cc5)cc4)cc3)cc2)CCCC1]

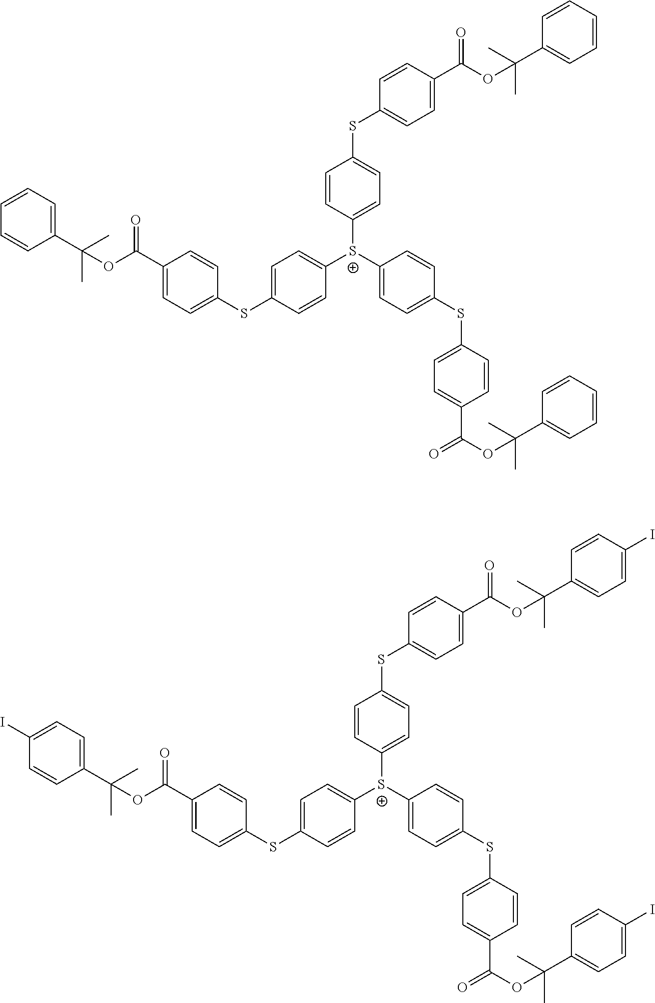 [SMILES: CC(C)(OC(=O)c1ccc(Sc2ccc([S+](c3ccc(Sc4ccc(C(=O)OC(C)(C)c5ccccc5)cc4)cc3)c3ccc(Sc4ccc(C(=O)OC(C)(C)c5ccccc5)cc4)cc3)cc2)cc1)c1ccccc1 ; CC(C)(OC(=O)c1ccc(Sc2ccc([S+](c3ccc(Sc4ccc(C(=O)OC(C)(C)c5ccc(I)cc5)cc4)cc3)c3ccc(Sc4ccc(C(=O)OC(C)(C)c5ccc(I)cc5)cc4)cc3)cc2)cc1)c1ccc(I)cc1]

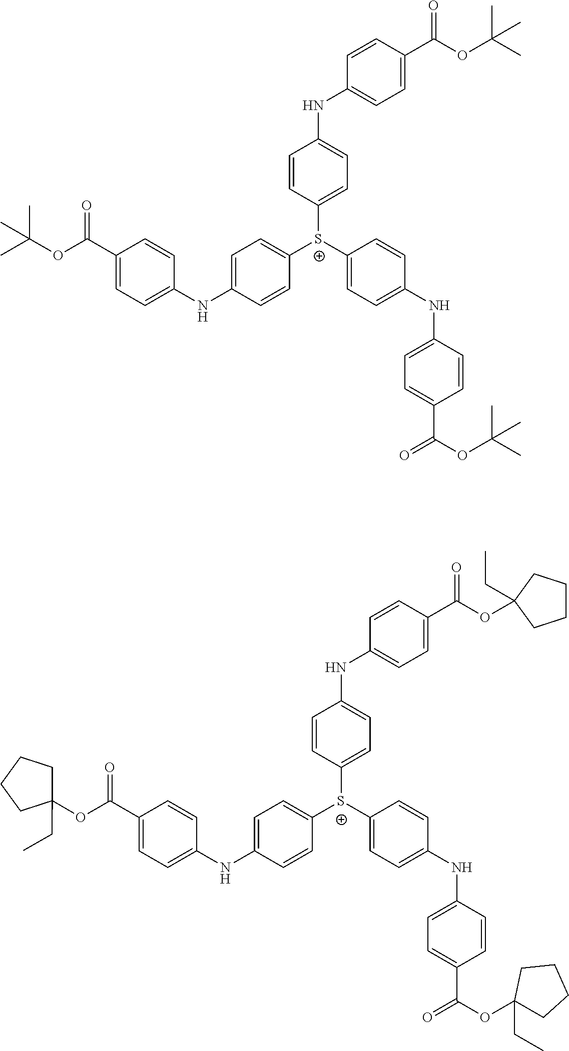 [SMILES: CC(C)(C)OC(=O)c1ccc(Nc2ccc([S+](c3ccc(Nc4ccc(C(=O)OC(C)(C)C)cc4)cc3)c3ccc(Nc4ccc(C(=O)OC(C)(C)C)cc4)cc3)cc2)cc1 ; CCC1(OC(=O)c2ccc(Nc3ccc([S+](c4ccc(Nc5ccc(C(=O)OC6(CC)CCCC6)cc5)cc4)c4ccc(Nc5ccc(C(=O)OC6(CC)CCCC6)cc5)cc4)cc3)cc2)CCCC1]

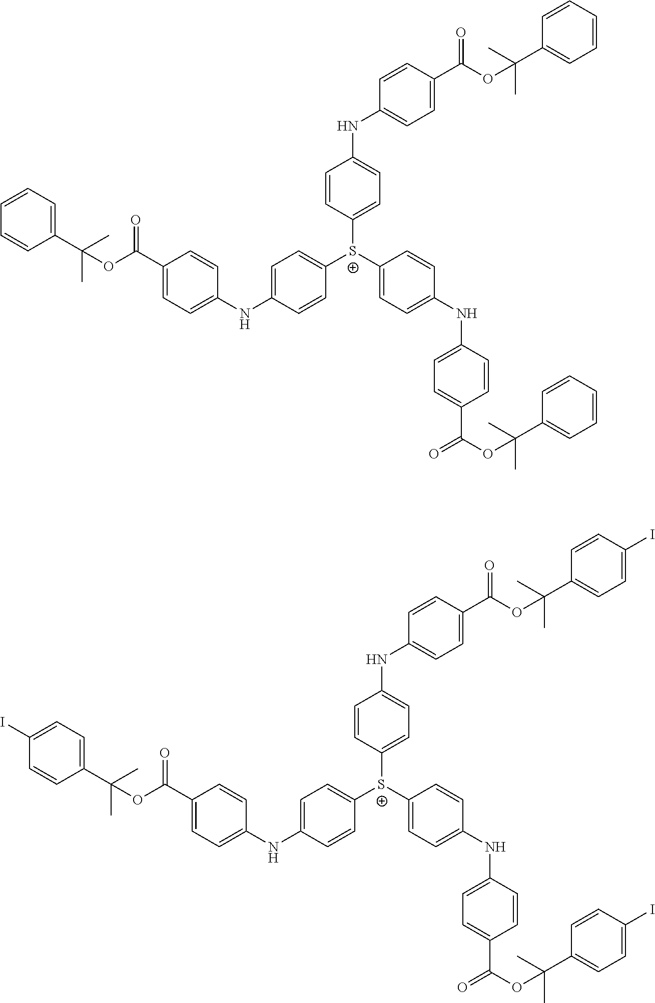 [SMILES: CC(C)(OC(=O)c1ccc(Nc2ccc([S+](c3ccc(Nc4ccc(C(=O)OC(C)(C)c5ccccc5)cc4)cc3)c3ccc(Nc4ccc(C(=O)OC(C)(C)c5ccccc5)cc4)cc3)cc2)cc1)c1ccccc1 ; CC(C)(OC(=O)c1ccc(Nc2ccc([S+](c3ccc(Nc4ccc(C(=O)OC(C)(C)c5ccc(I)cc5)cc4)cc3)c3ccc(Nc4ccc(C(=O)OC(C)(C)c5ccc(I)cc5)cc4)cc3)cc2)cc1)c1ccc(I)cc1]

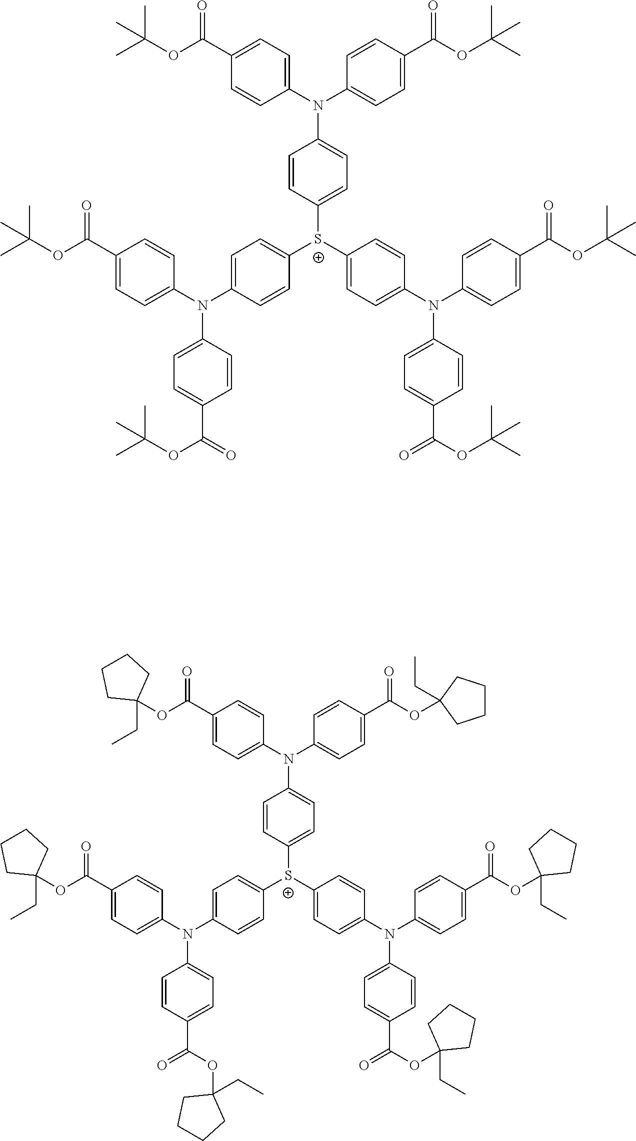 [SMILES: CC(C)(C)OC(=O)c1ccc(N(c2ccc(C(=O)OC(C)(C)C)cc2)c2ccc([S+](c3ccc(N(c4ccc(C(=O)OC(C)(C)C)cc4)c4ccc(C(=O)OC(C)(C)C)cc4)cc3)c3ccc(N(c4ccc(C(=O)OC(C)(C)C)cc4)c4ccc(C(=O)OC(C)(C)C)cc4)cc3)cc2)cc1 ; CCC1(OC(=O)c2ccc(N(c3ccc(C(=O)OC4(CC)CCCC4)cc3)c3ccc([S+](c4ccc(N(c5ccc(C(=O)OC6(CC)CCCC6)cc5)c5ccc(C(=O)OC6(CC)CCCC6)cc5)cc4)c4ccc(N(c5ccc(C(=O)OC6(CC)CCCC6)cc5)c5ccc(C(=O)OC6(CC)CCCC6)cc5)cc4)cc3)cc2)CCCC1]

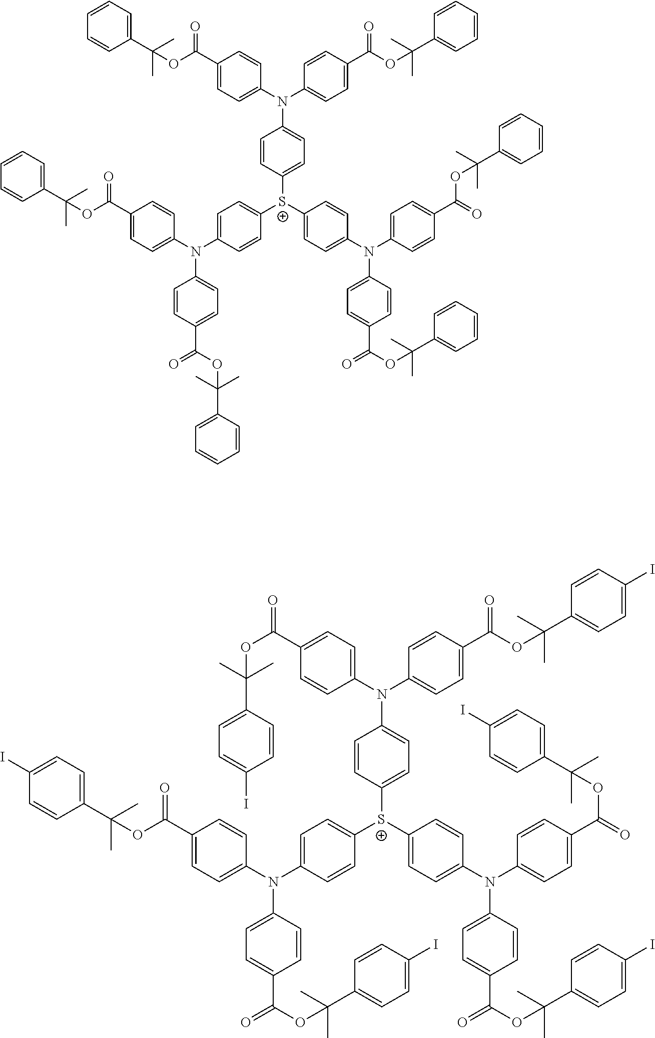 [SMILES: CC(C)(OC(=O)c1ccc(N(c2ccc(C(=O)OC(C)(C)c3ccccc3)cc2)c2ccc([S+](c3ccc(N(c4ccc(C(=O)OC(C)(C)c5ccccc5)cc4)c4ccc(C(=O)OC(C)(C)c5ccccc5)cc4)cc3)c3ccc(N(c4ccc(C(=O)OC(C)(C)c5ccccc5)cc4)c4ccc(C(=O)OC(C)(C)c5ccccc5)cc4)cc3)cc2)cc1)c1ccccc1 ; CC(C)(OC(=O)c1ccc(N(c2ccc(C(=O)OC(C)(C)c3ccc(I)cc3)cc2)c2ccc([S+](c3ccc(N(c4ccc(C(=O)OC(C)(C)c5ccc(I)cc5)cc4)c4ccc(C(=O)OC(C)(C)c5ccc(I)cc5)cc4)cc3)c3ccc(N(c4ccc(C(=O)OC(C)(C)c5ccc(I)cc5)cc4)c4ccc(C(=O)OC(C)(C)c5ccc(I)cc5)cc4)cc3)cc2)cc1)c1ccc(I)cc1]

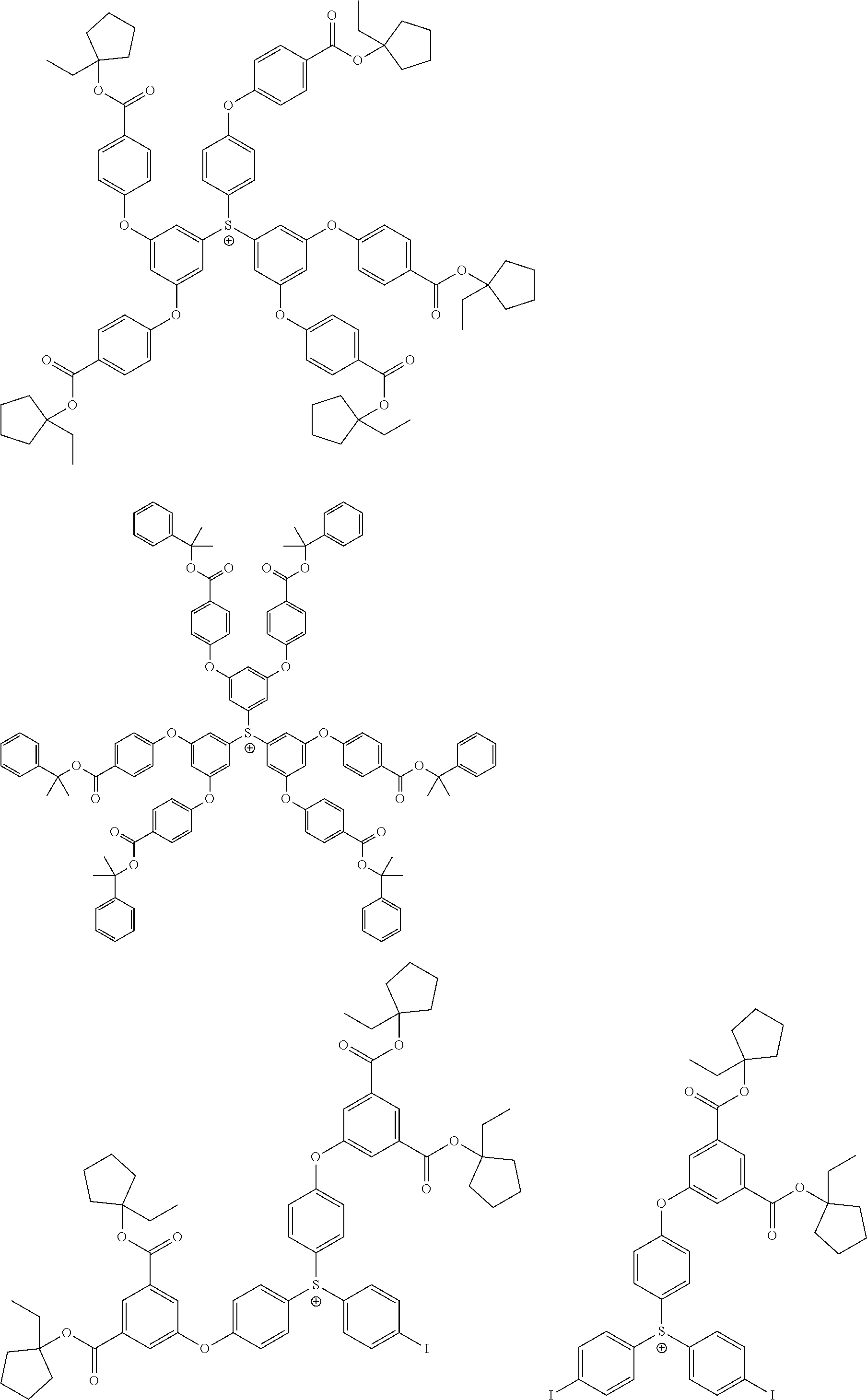

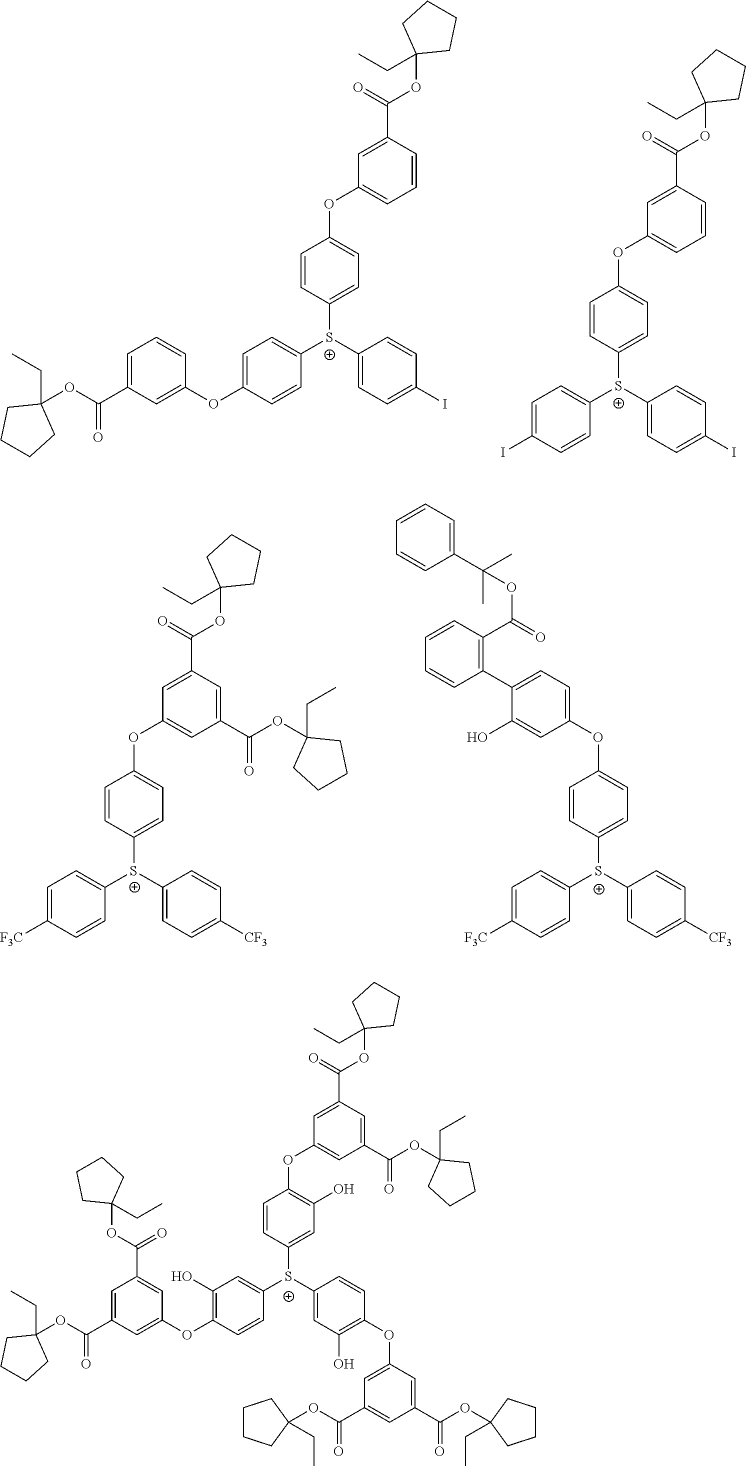

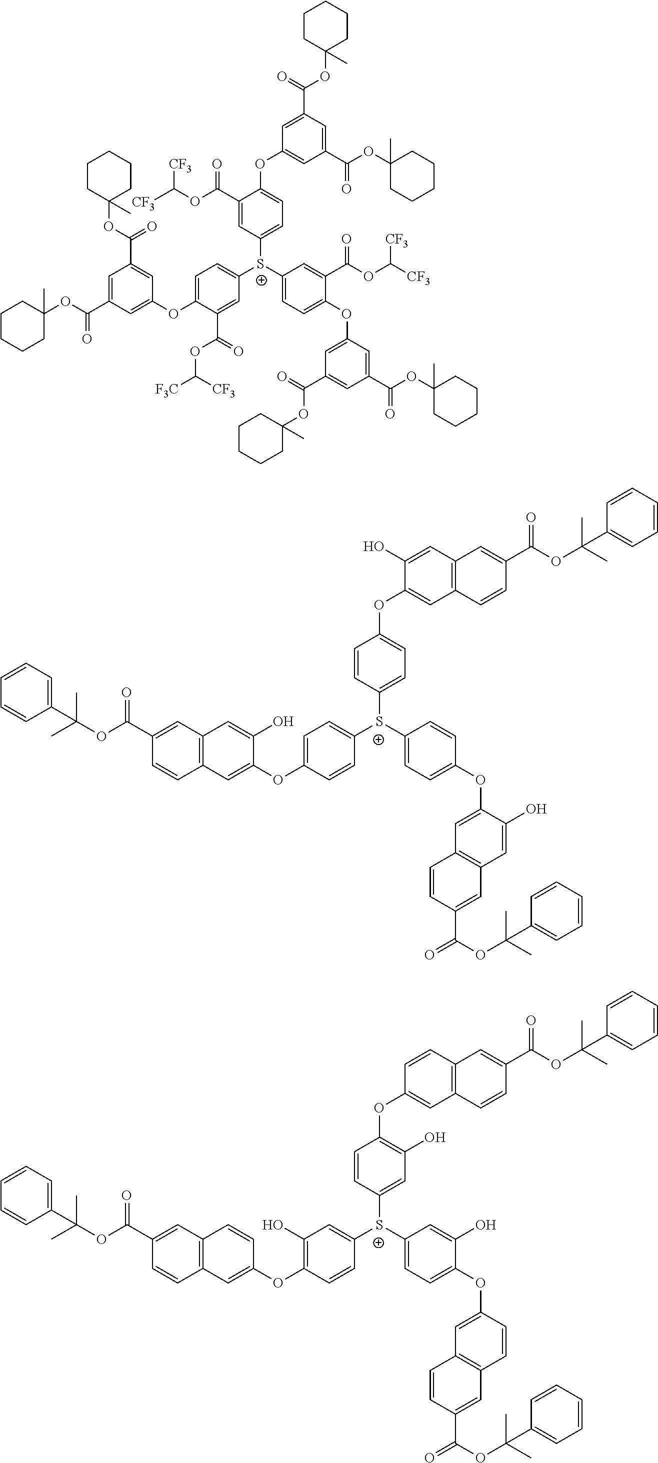

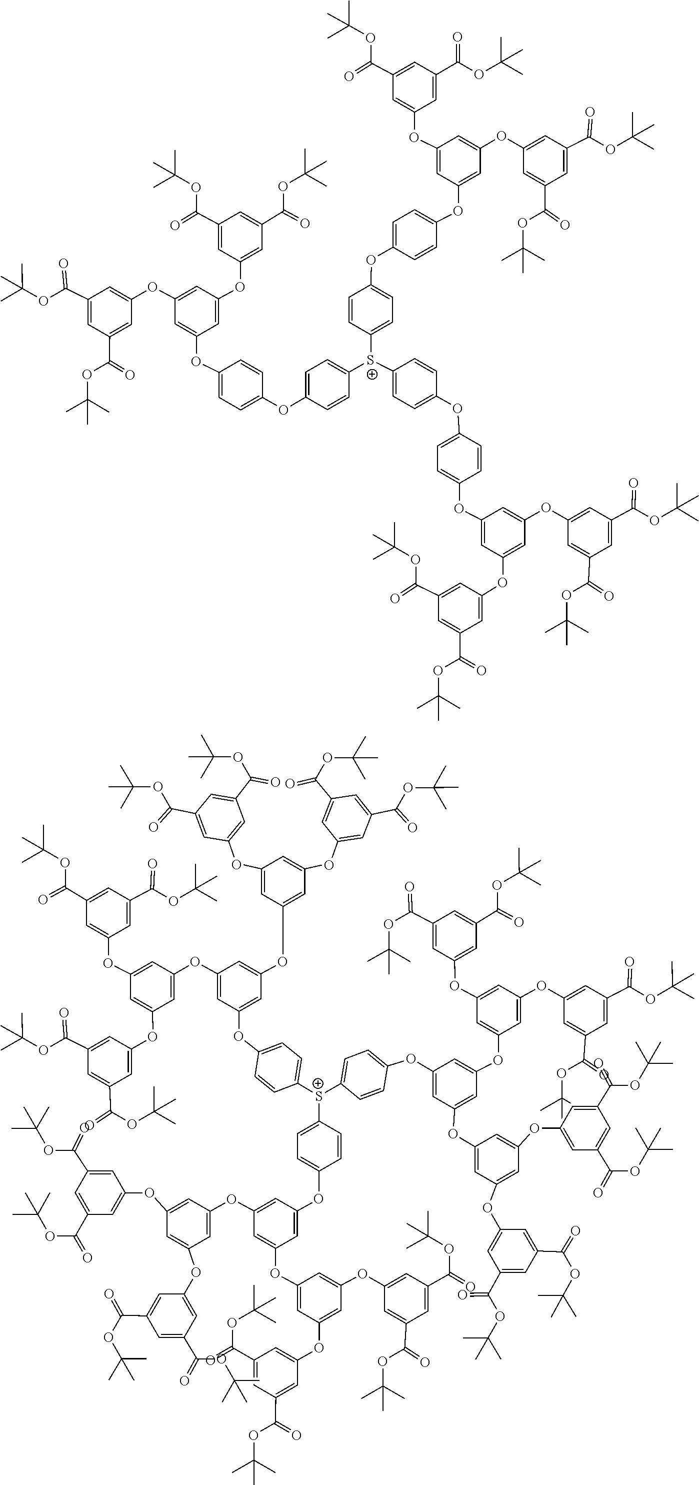

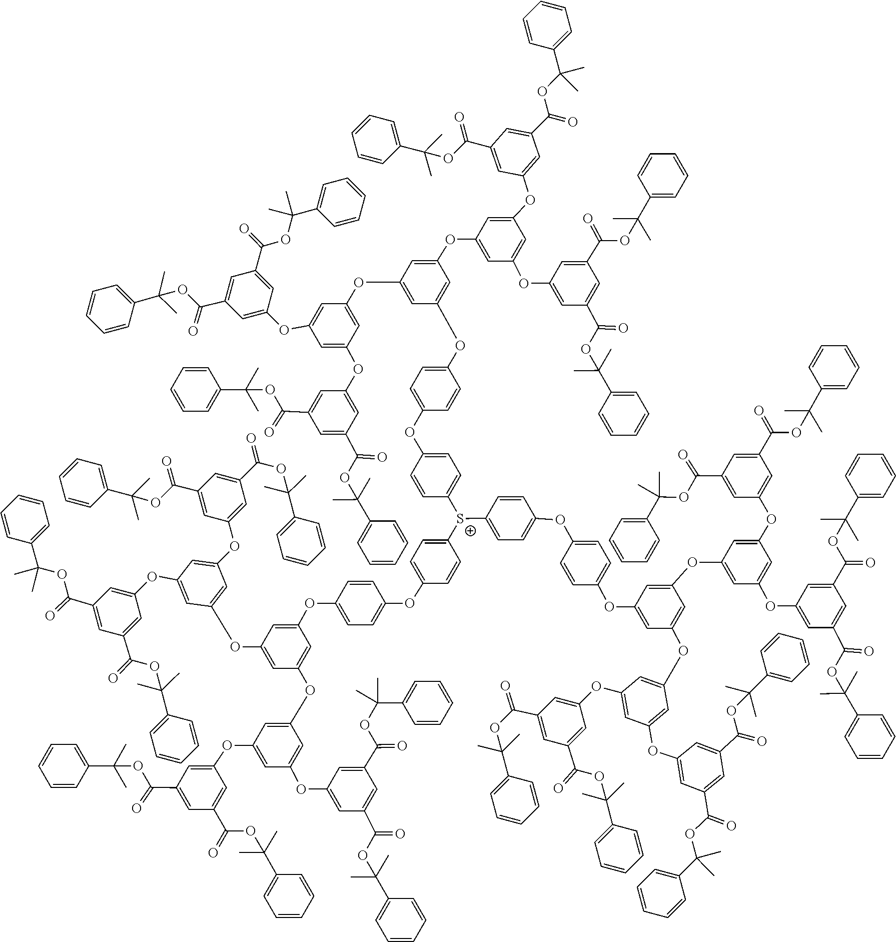

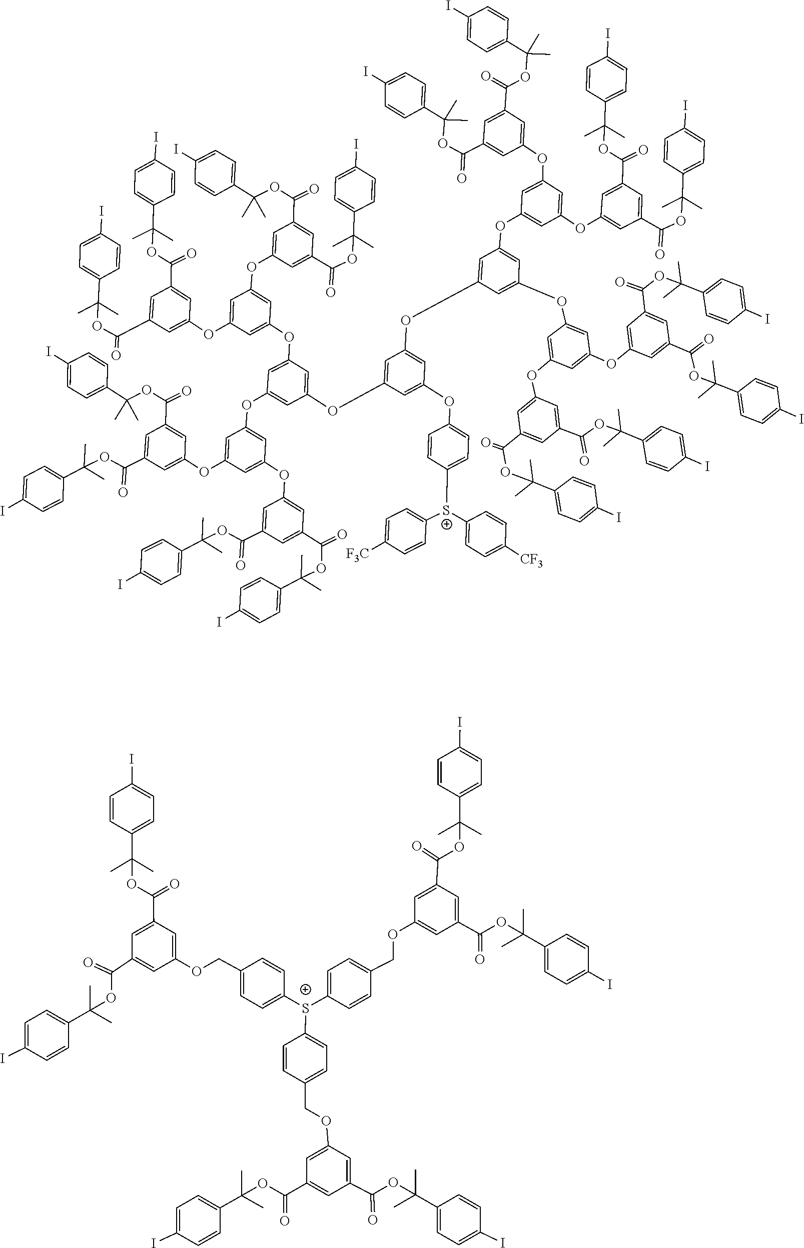

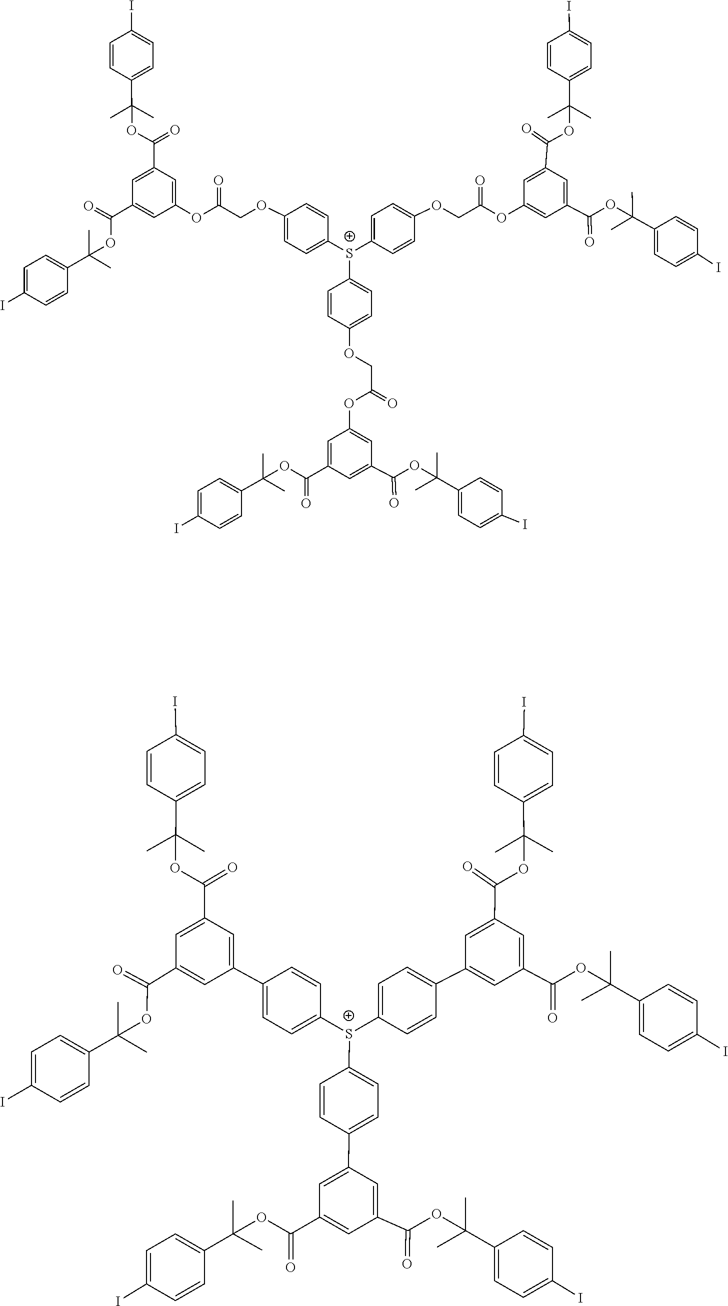 [SMILES: CC(C)(OC(=O)c1cc(OC(=O)COc2ccc([S+](c3ccc(OCC(=O)Oc4cc(C(=O)OC(C)(C)c5ccc(I)cc5)cc(C(=O)OC(C)(C)c5ccc(I)cc5)c4)cc3)c3ccc(OCC(=O)Oc4cc(C(=O)OC(C)(C)c5ccc(I)cc5)cc(C(=O)OC(C)(C)c5ccc(I)cc5)c4)cc3)cc2)cc(C(=O)OC(C)(C)c2ccc(I)cc2)c1)c1ccc(I)cc1 ; CC(C)(OC(=O)c1cc(C(=O)OC(C)(C)c2ccc(I)cc2)cc(-c2ccc([S+](c3ccc(-c4cc(C(=O)OC(C)(C)c5ccc(I)cc5)cc(C(=O)OC(C)(C)c5ccc(I)cc5)c4)cc3)c3ccc(-c4cc(C(=O)OC(C)(C)c5ccc(I)cc5)cc(C(=O)OC(C)(C)c5ccc(I)cc5)c4)cc3)cc2)c1)c1ccc(I)cc1]

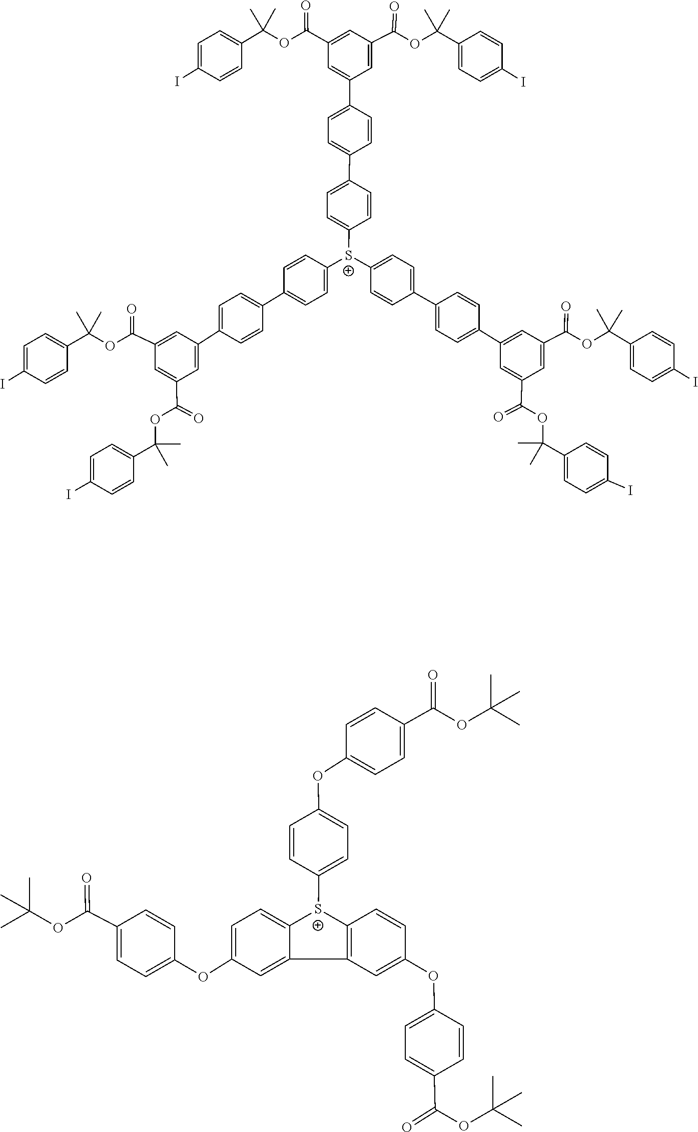 [SMILES: CC(C)(OC(=O)c1cc(C(=O)OC(C)(C)c2ccc(I)cc2)cc(-c2ccc(-c3ccc([S+](c4ccc(-c5ccc(-c6cc(C(=O)OC(C)(C)c7ccc(I)cc7)cc(C(=O)OC(C)(C)c7ccc(I)cc7)c6)cc5)cc4)c4ccc(-c5ccc(-c6cc(C(=O)OC(C)(C)c7ccc(I)cc7)cc(C(=O)OC(C)(C)c7ccc(I)cc7)c6)cc5)cc4)cc3)cc2)c1)c1ccc(I)cc1 ; CC(C)(C)OC(=O)c1ccc(Oc2ccc(-[s+]3c4ccc(Oc5ccc(C(=O)OC(C)(C)C)cc5)cc4c4cc(Oc5ccc(C(=O)OC(C)(C)C)cc5)ccc43)cc2)cc1]

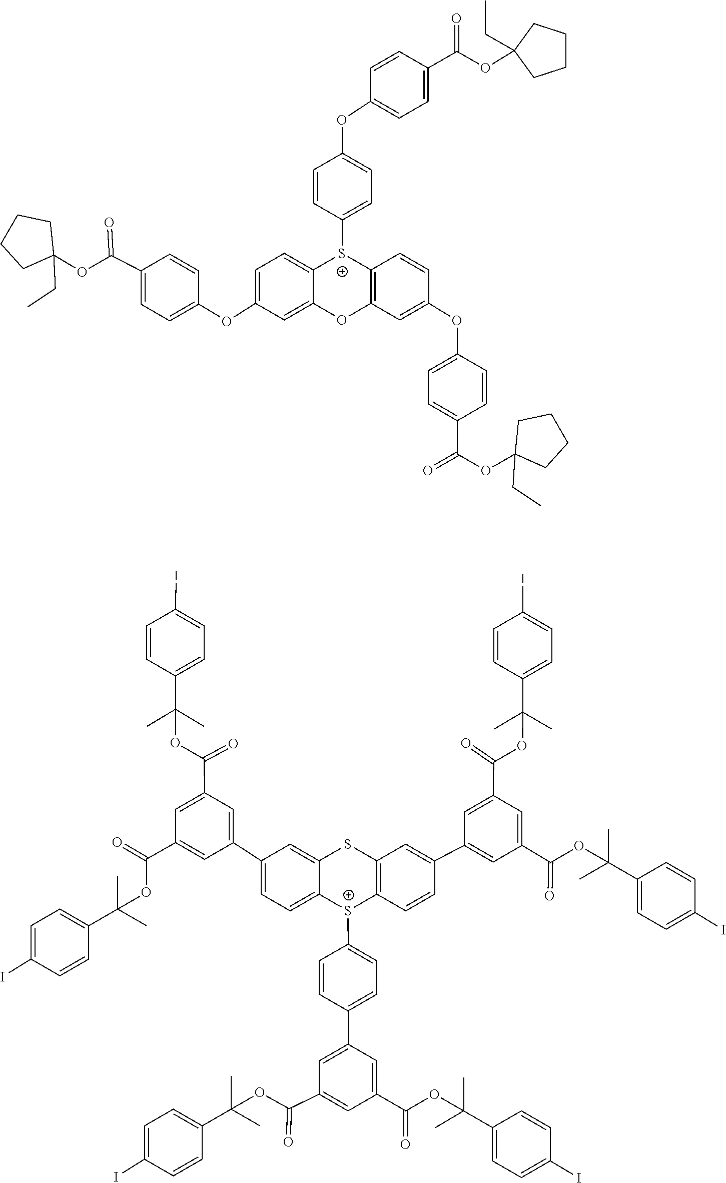 [SMILES: CCC1(OC(=O)c2ccc(Oc3ccc([S+]4c5ccc(Oc6ccc(C(=O)OC7(CC)CCCC7)cc6)cc5Oc5cc(Oc6ccc(C(=O)OC7(CC)CCCC7)cc6)ccc54)cc3)cc2)CCCC1 ; CC(C)(OC(=O)c1cc(C(=O)OC(C)(C)c2ccc(I)cc2)cc(-c2ccc([S+]3c4ccc(-c5cc(C(=O)OC(C)(C)c6ccc(I)cc6)cc(C(=O)OC(C)(C)c6ccc(I)cc6)c5)cc4Sc4cc(-c5cc(C(=O)OC(C)(C)c6ccc(I)cc6)cc(C(=O)OC(C)(C)c6ccc(I)cc6)c5)ccc43)cc2)c1)c1ccc(I)cc1]

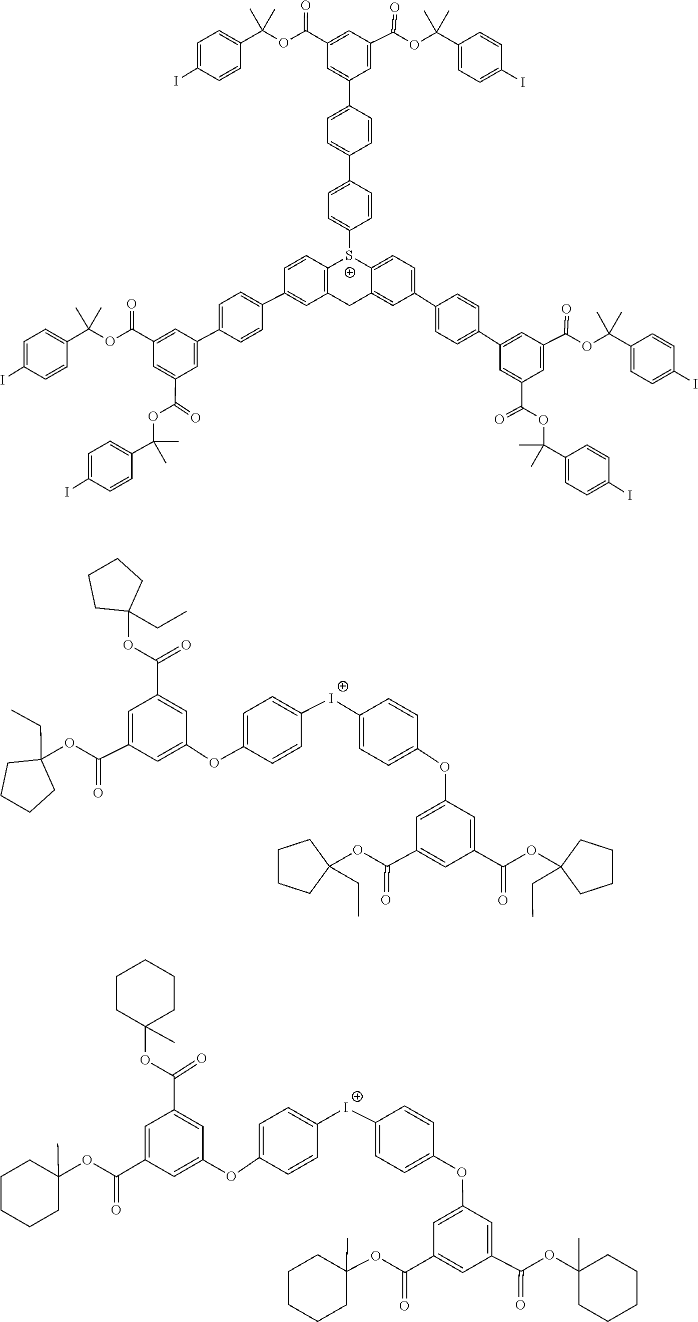 [SMILES: CC(C)(OC(=O)c1cc(C(=O)OC(C)(C)c2ccc(I)cc2)cc(-c2ccc(-c3ccc([S+]4c5ccc(-c6ccc(-c7cc(C(=O)OC(C)(C)c8ccc(I)cc8)cc(C(=O)OC(C)(C)c8ccc(I)cc8)c7)cc6)cc5Cc5cc(-c6ccc(-c7cc(C(=O)OC(C)(C)c8ccc(I)cc8)cc(C(=O)OC(C)(C)c8ccc(I)cc8)c7)cc6)ccc54)cc3)cc2)c1)c1ccc(I)cc1 ; CCC1(OC(=O)c2cc(Oc3ccc([I+]c4ccc(Oc5cc(C(=O)OC6(CC)CCCC6)cc(C(=O)OC6(CC)CCCC6)c5)cc4)cc3)cc(C(=O)OC3(CC)CCCC3)c2)CCCC1 ; CC1(OC(=O)c2cc(Oc3ccc([I+]c4ccc(Oc5cc(C(=O)OC6(C)CCCCC6)cc(C(=O)OC6(C)CCCCC6)c5)cc4)cc3)cc(C(=O)OC3(C)CCCCC3)c2)CCCCC1]

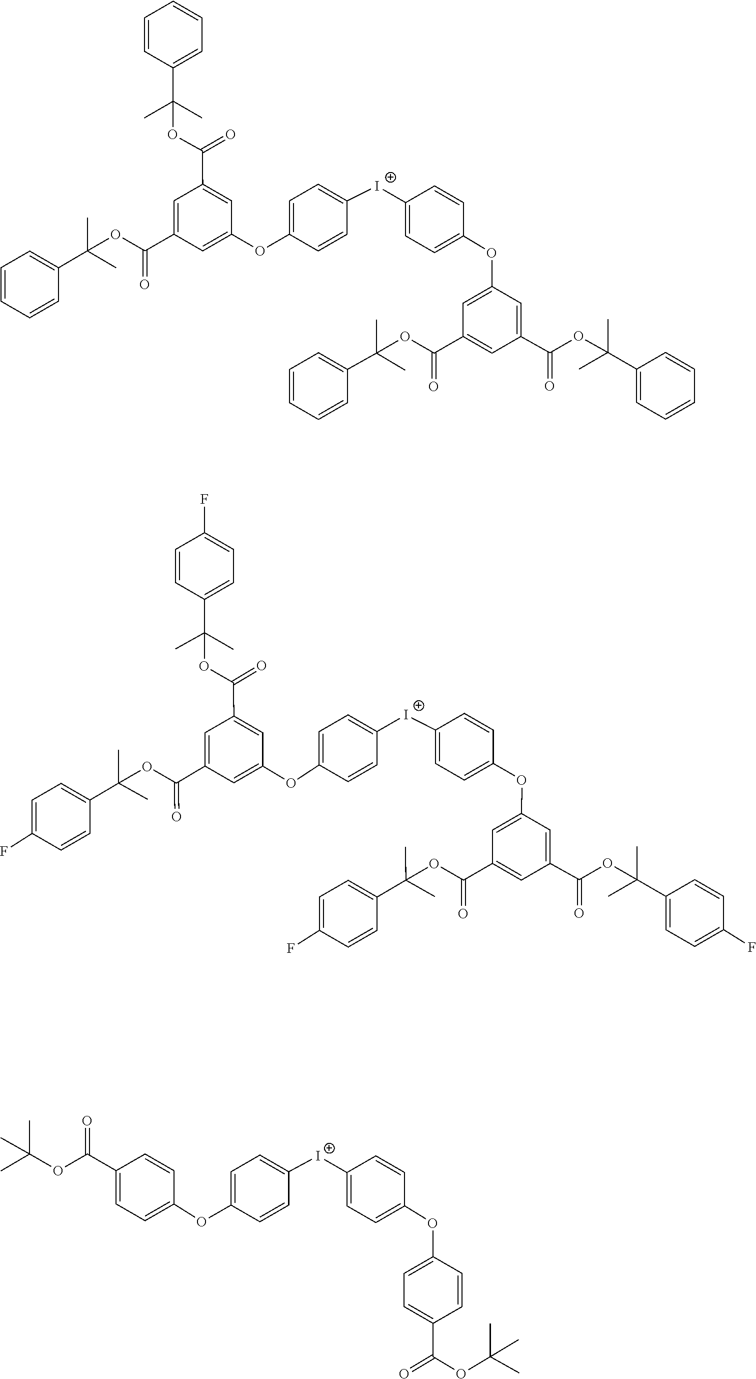 [SMILES: CC(C)(OC(=O)c1cc(Oc2ccc([I+]c3ccc(Oc4cc(C(=O)OC(C)(C)c5ccccc5)cc(C(=O)OC(C)(C)c5ccccc5)c4)cc3)cc2)cc(C(=O)OC(C)(C)c2ccccc2)c1)c1ccccc1 ; CC(C)(OC(=O)c1cc(Oc2ccc([I+]c3ccc(Oc4cc(C(=O)OC(C)(C)c5ccc(F)cc5)cc(C(=O)OC(C)(C)c5ccc(F)cc5)c4)cc3)cc2)cc(C(=O)OC(C)(C)c2ccc(F)cc2)c1)c1ccc(F)cc1 ; CC(C)(C)OC(=O)c1ccc(Oc2ccc([I+]c3ccc(Oc4ccc(C(=O)OC(C)(C)C)cc4)cc3)cc2)cc1]

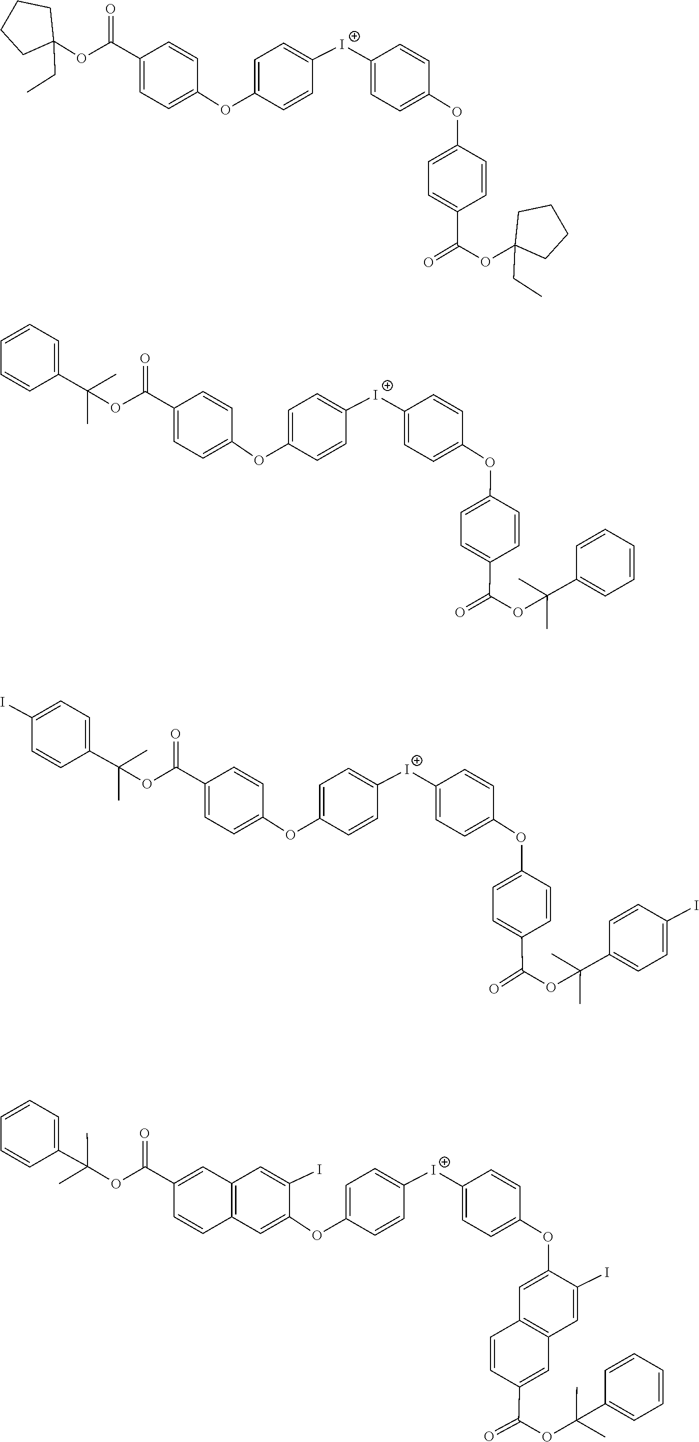 [SMILES: CCC1(OC(=O)c2ccc(Oc3ccc([I+]c4ccc(Oc5ccc(C(=O)OC6(CC)CCCC6)cc5)cc4)cc3)cc2)CCCC1 ; CC(C)(OC(=O)c1ccc(Oc2ccc([I+]c3ccc(Oc4ccc(C(=O)OC(C)(C)c5ccccc5)cc4)cc3)cc2)cc1)c1ccccc1 ; CC(C)(OC(=O)c1ccc(Oc2ccc([I+]c3ccc(Oc4ccc(C(=O)OC(C)(C)c5ccc(I)cc5)cc4)cc3)cc2)cc1)c1ccc(I)cc1 ; CC(C)(OC(=O)c1ccc2cc(Oc3ccc([I+]c4ccc(Oc5cc6ccc(C(=O)OC(C)(C)c7ccccc7)cc6cc5I)cc4)cc3)c(I)cc2c1)c1ccccc1]

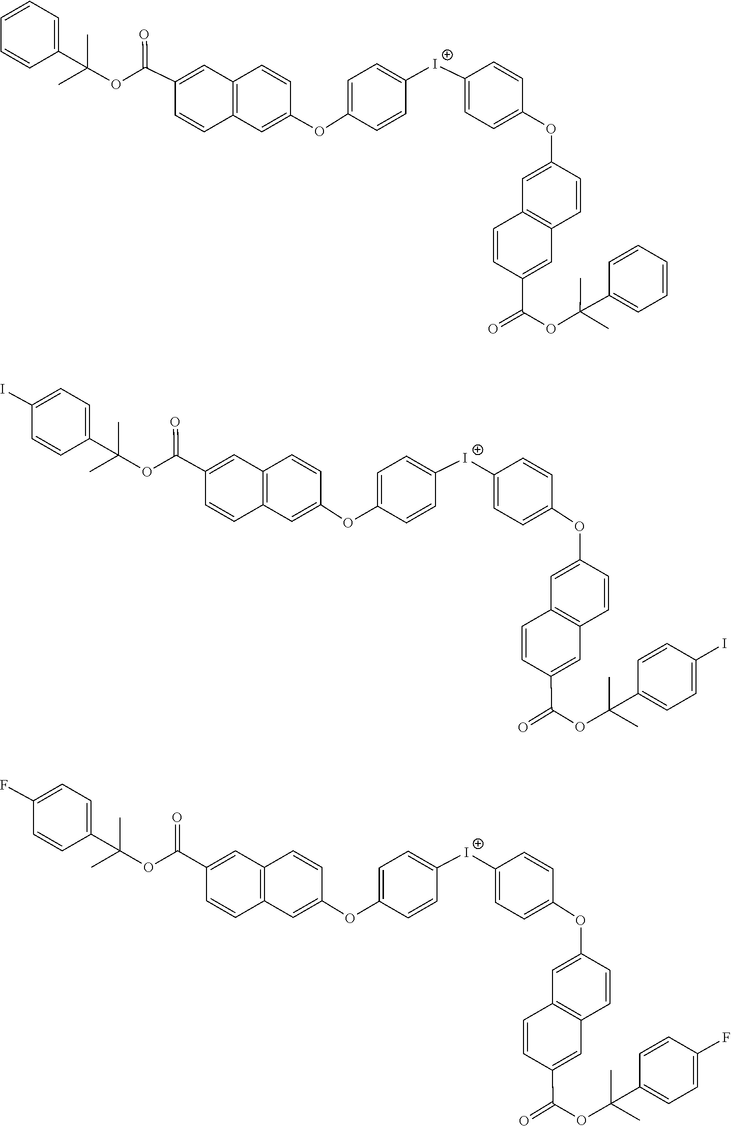 [SMILES: CC(C)(OC(=O)c1ccc2cc(Oc3ccc([I+]c4ccc(Oc5ccc6cc(C(=O)OC(C)(C)c7ccccc7)ccc6c5)cc4)cc3)ccc2c1)c1ccccc1 ; CC(C)(OC(=O)c1ccc2cc(Oc3ccc([I+]c4ccc(Oc5ccc6cc(C(=O)OC(C)(C)c7ccc(I)cc7)ccc6c5)cc4)cc3)ccc2c1)c1ccc(I)cc1 ; CC(C)(OC(=O)c1ccc2cc(Oc3ccc([I+]c4ccc(Oc5ccc6cc(C(=O)OC(C)(C)c7ccc(F)cc7)ccc6c5)cc4)cc3)ccc2c1)c1ccc(F)cc1]

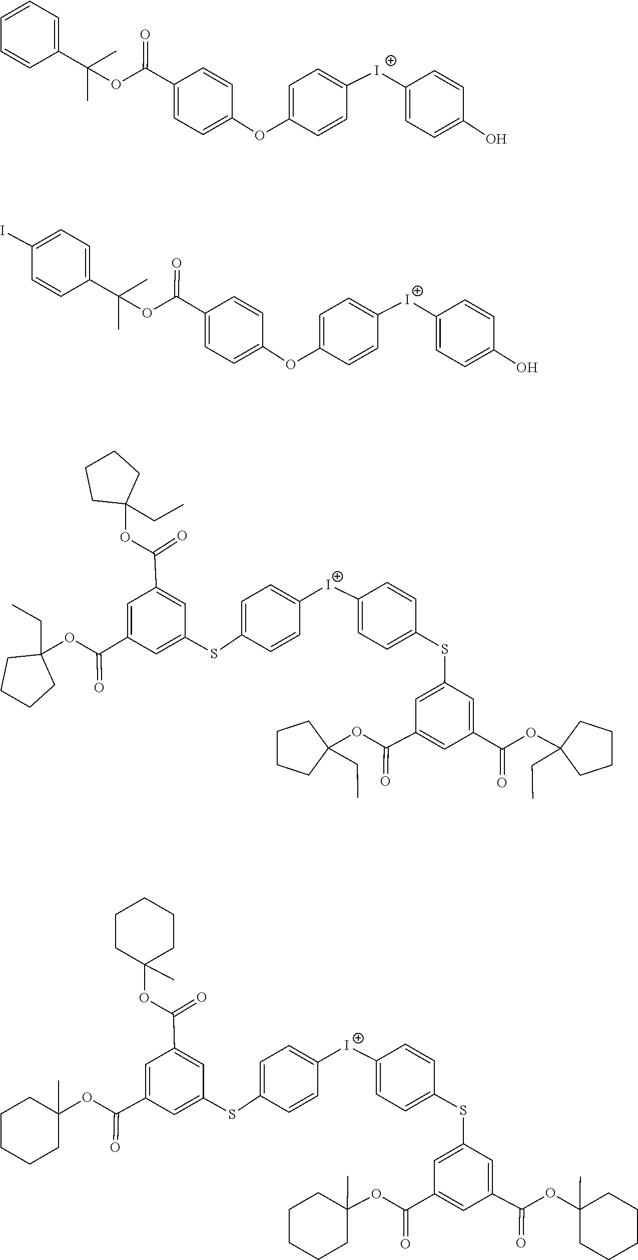 [SMILES: CC(C)(OC(=O)c1ccc(Oc2ccc([I+]c3ccc(O)cc3)cc2)cc1)c1ccccc1 ; CC(C)(OC(=O)c1ccc(Oc2ccc([I+]c3ccc(O)cc3)cc2)cc1)c1ccc(I)cc1 ; CCC1(OC(=O)c2cc(Sc3ccc([I+]c4ccc(Sc5cc(C(=O)OC6(CC)CCCC6)cc(C(=O)OC6(CC)CCCC6)c5)cc4)cc3)cc(C(=O)OC3(CC)CCCC3)c2)CCCC1 ; CC1(OC(=O)c2cc(Sc3ccc([I+]c4ccc(Sc5cc(C(=O)OC6(C)CCCCC6)cc(C(=O)OC6(C)CCCCC6)c5)cc4)cc3)cc(C(=O)OC3(C)CCCCC3)c2)CCCCC1]

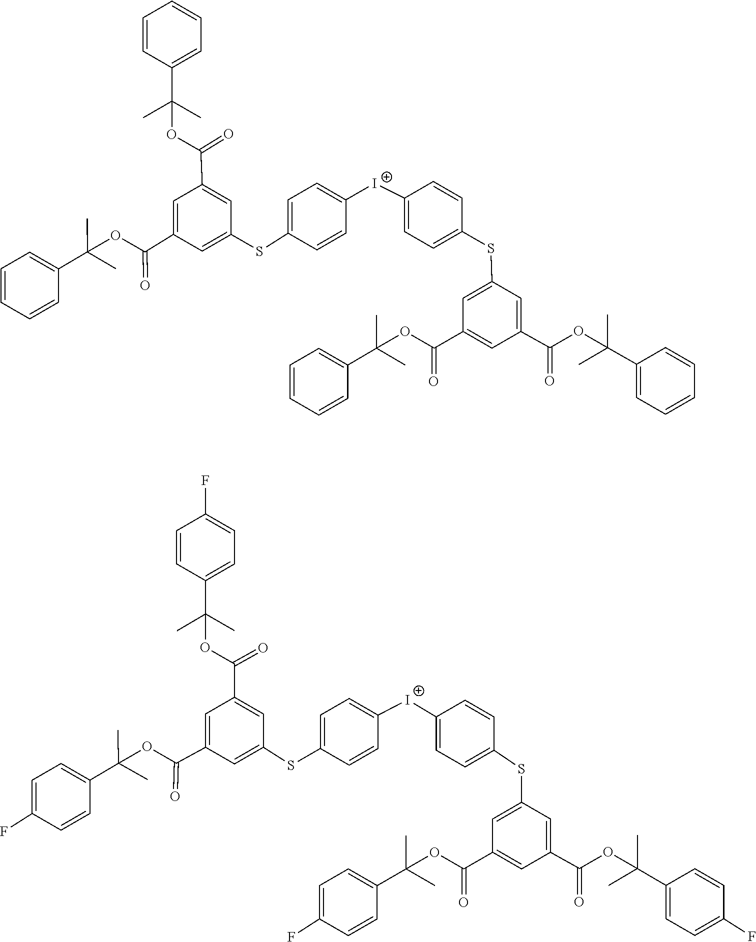 [SMILES: CC(C)(OC(=O)c1cc(Sc2ccc([I+]c3ccc(Sc4cc(C(=O)OC(C)(C)c5ccccc5)cc(C(=O)OC(C)(C)c5ccccc5)c4)cc3)cc2)cc(C(=O)OC(C)(C)c2ccccc2)c1)c1ccccc1 ; CC(C)(OC(=O)c1cc(Sc2ccc([I+]c3ccc(Sc4cc(C(=O)OC(C)(C)c5ccc(F)cc5)cc(C(=O)OC(C)(C)c5ccc(F)cc5)c4)cc3)cc2)cc(C(=O)OC(C)(C)c2ccc(F)cc2)c1)c1ccc(F)cc1]

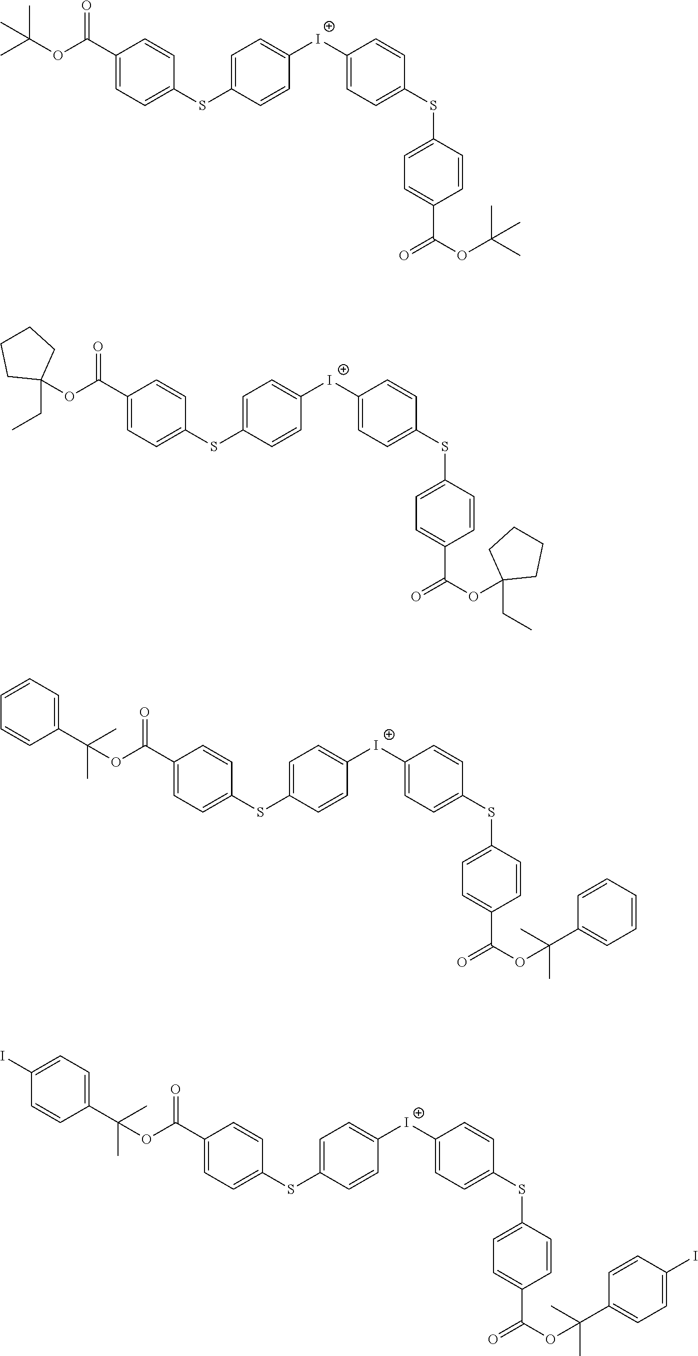 [SMILES: CC(C)(C)OC(=O)c1ccc(Sc2ccc([I+]c3ccc(Sc4ccc(C(=O)OC(C)(C)C)cc4)cc3)cc2)cc1 ; CCC1(OC(=O)c2ccc(Sc3ccc([I+]c4ccc(Sc5ccc(C(=O)OC6(CC)CCCC6)cc5)cc4)cc3)cc2)CCCC1 ; CC(C)(OC(=O)c1ccc(Sc2ccc([I+]c3ccc(Sc4ccc(C(=O)OC(C)(C)c5ccccc5)cc4)cc3)cc2)cc1)c1ccccc1 ; CC(C)(OC(=O)c1ccc(Sc2ccc([I+]c3ccc(Sc4ccc(C(=O)OC(C)(C)c5ccc(I)cc5)cc4)cc3)cc2)cc1)c1ccc(I)cc1]

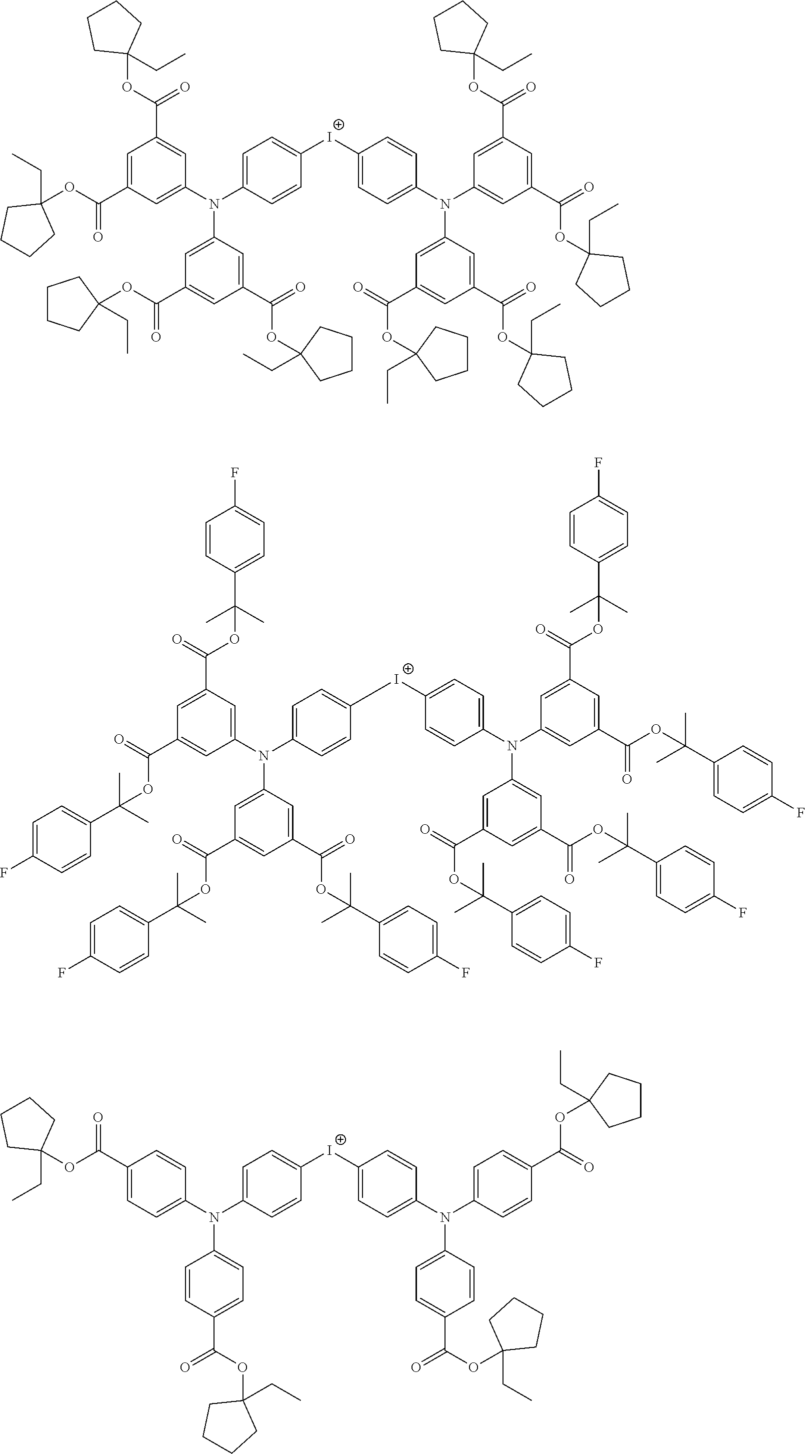

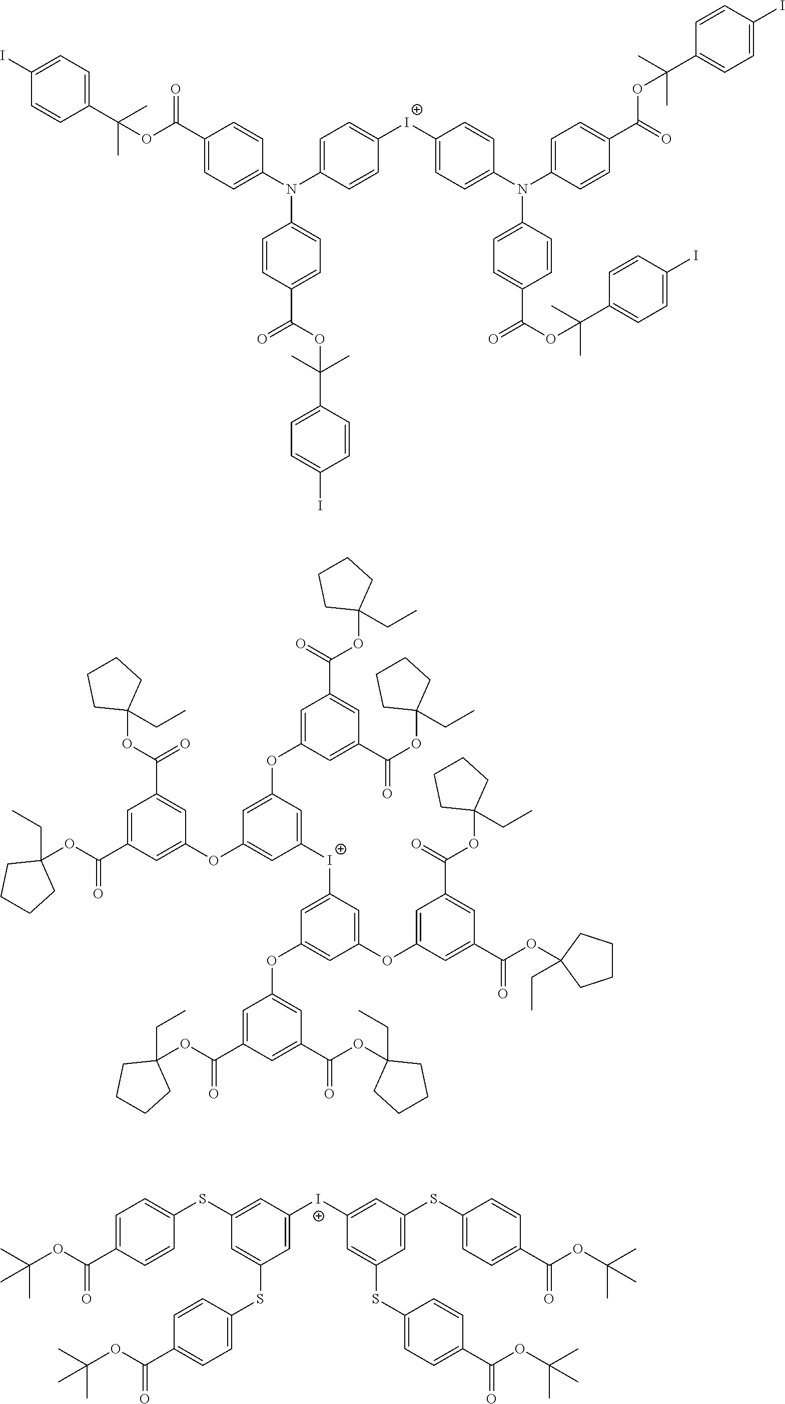

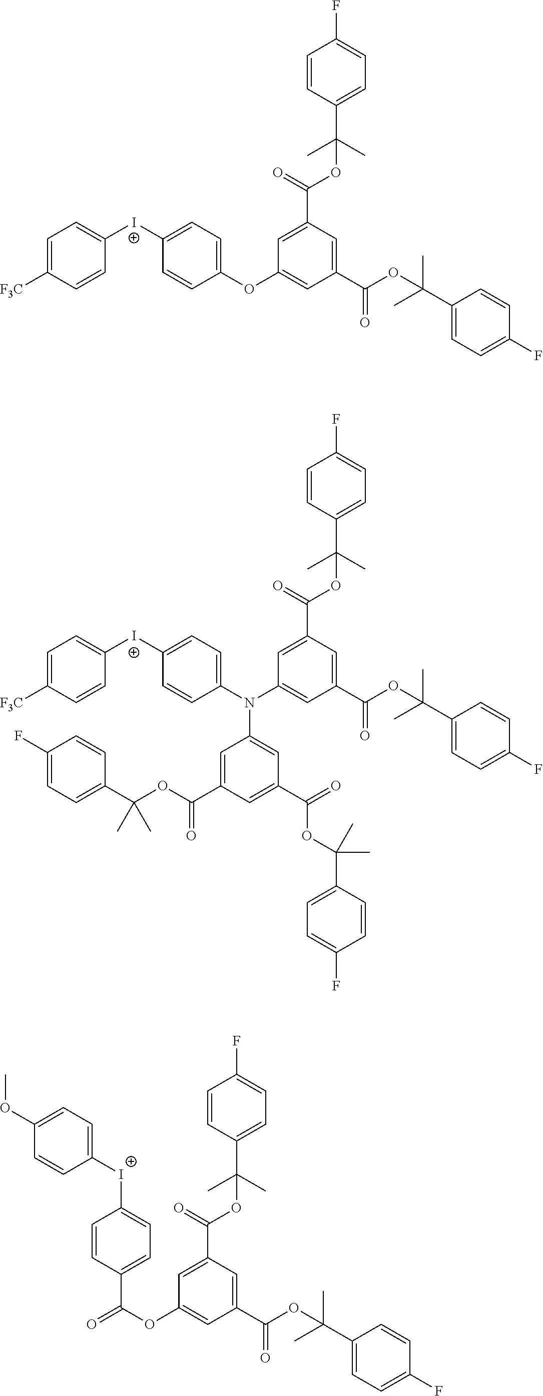 [SMILES: CC(C)(OC(=O)c1cc(Oc2ccc([I+]c3ccc(C(F)(F)F)cc3)cc2)cc(C(=O)OC(C)(C)c2ccc(F)cc2)c1)c1ccc(F)cc1 ; CC(C)(OC(=O)c1cc(C(=O)OC(C)(C)c2ccc(F)cc2)cc(N(c2ccc([I+]c3ccc(C(F)(F)F)cc3)cc2)c2cc(C(=O)OC(C)(C)c3ccc(F)cc3)cc(C(=O)OC(C)(C)c3ccc(F)cc3)c2)c1)c1ccc(F)cc1 ; COc1ccc([I+]c2ccc(C(=O)Oc3cc(C(=O)OC(C)(C)c4ccc(F)cc4)cc(C(=O)OC(C)(C)c4ccc(F)cc4)c3)cc2)cc1]

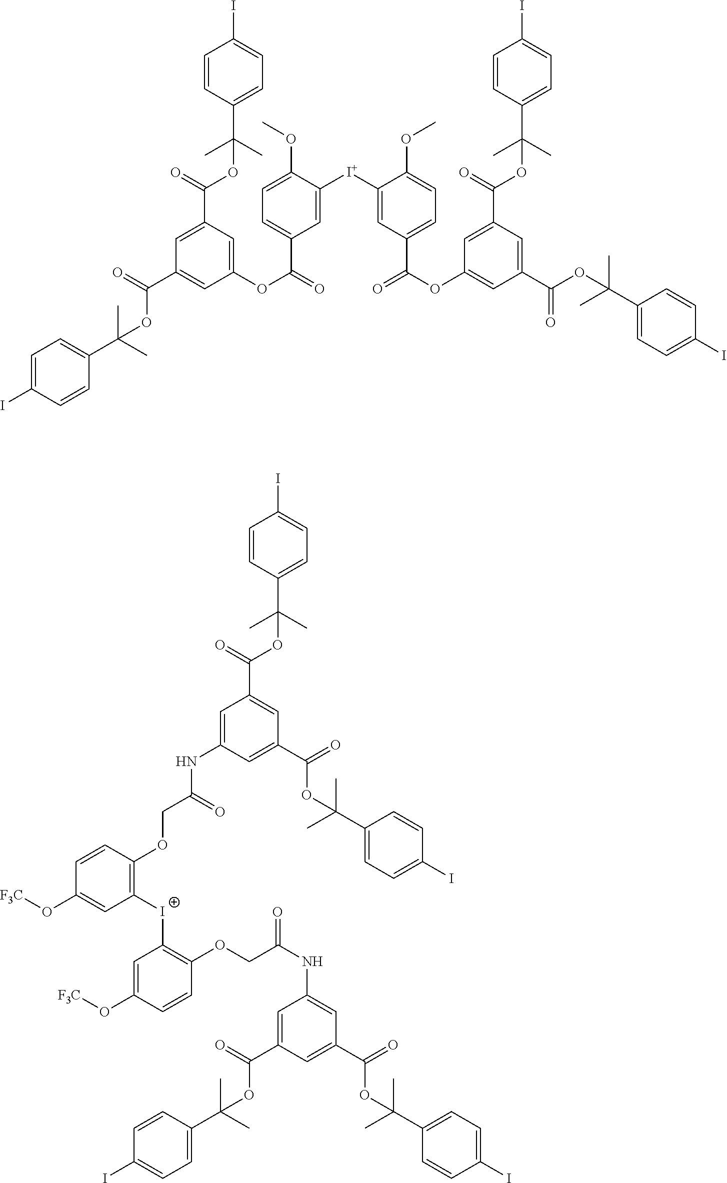 [SMILES: COc1ccc(C(=O)Oc2cc(C(=O)OC(C)(C)c3ccc(I)cc3)cc(C(=O)OC(C)(C)c3ccc(I)cc3)c2)cc1[I+]c1cc(C(=O)Oc2cc(C(=O)OC(C)(C)c3ccc(I)cc3)cc(C(=O)OC(C)(C)c3ccc(I)cc3)c2)ccc1OC ; CC(C)(OC(=O)c1cc(NC(=O)COc2ccc(OC(F)(F)F)cc2[I+]c2cc(OC(F)(F)F)ccc2OCC(=O)Nc2cc(C(=O)OC(C)(C)c3ccc(I)cc3)cc(C(=O)OC(C)(C)c3ccc(I)cc3)c2)cc(C(=O)OC(C)(C)c2ccc(I)cc2)c1)c1ccc(I)cc1]

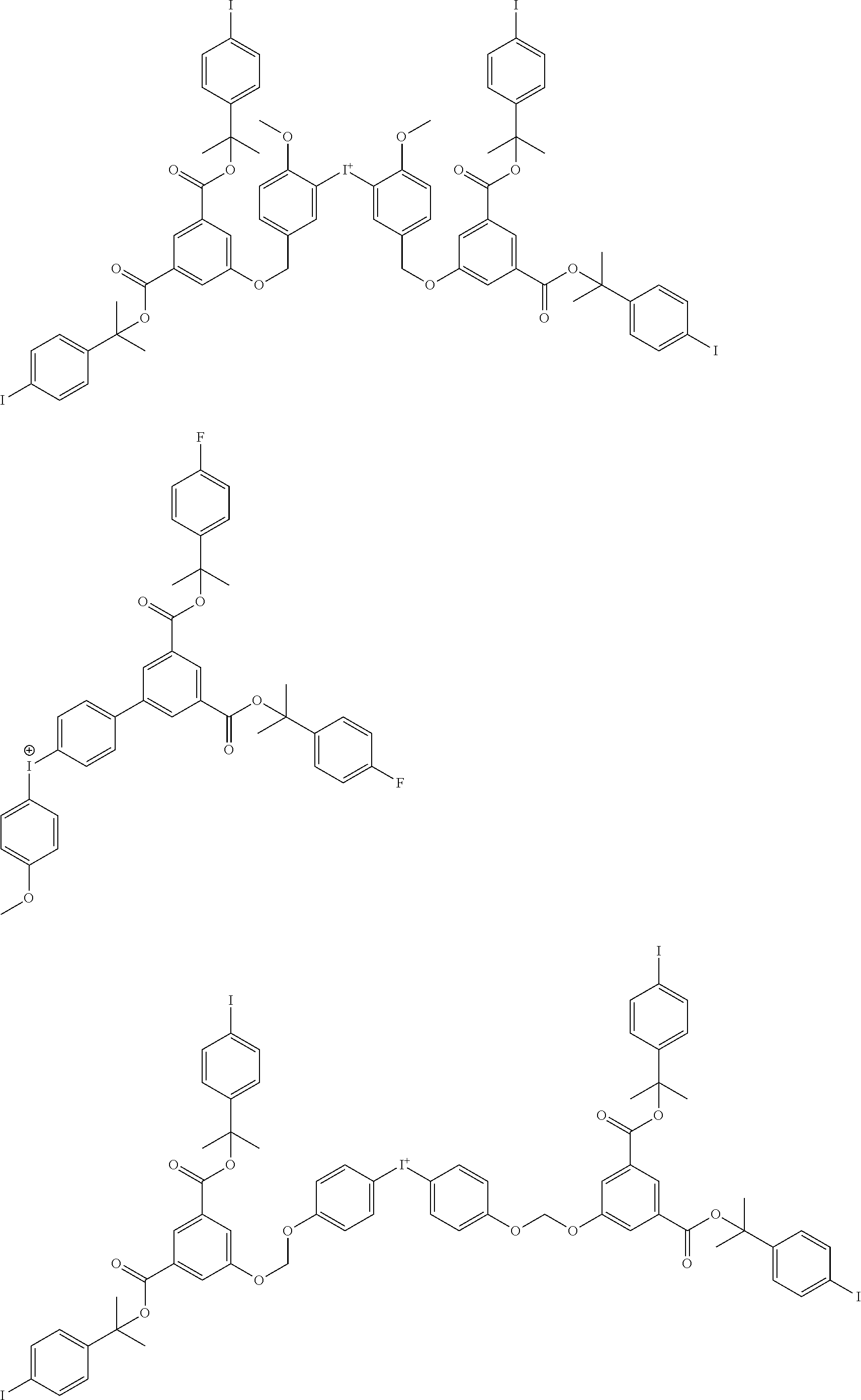 [SMILES: COc1ccc(COc2cc(C(=O)OC(C)(C)c3ccc(I)cc3)cc(C(=O)OC(C)(C)c3ccc(I)cc3)c2)cc1[I+]c1cc(COc2cc(C(=O)OC(C)(C)c3ccc(I)cc3)cc(C(=O)OC(C)(C)c3ccc(I)cc3)c2)ccc1OC ; COc1ccc([I+]c2ccc(-c3cc(C(=O)OC(C)(C)c4ccc(F)cc4)cc(C(=O)OC(C)(C)c4ccc(F)cc4)c3)cc2)cc1 ; CC(C)(OC(=O)c1cc(OCOc2ccc([I+]c3ccc(OCOc4cc(C(=O)OC(C)(C)c5ccc(I)cc5)cc(C(=O)OC(C)(C)c5ccc(I)cc5)c4)cc3)cc2)cc(C(=O)OC(C)(C)c2ccc(I)cc2)c1)c1ccc(I)cc1]

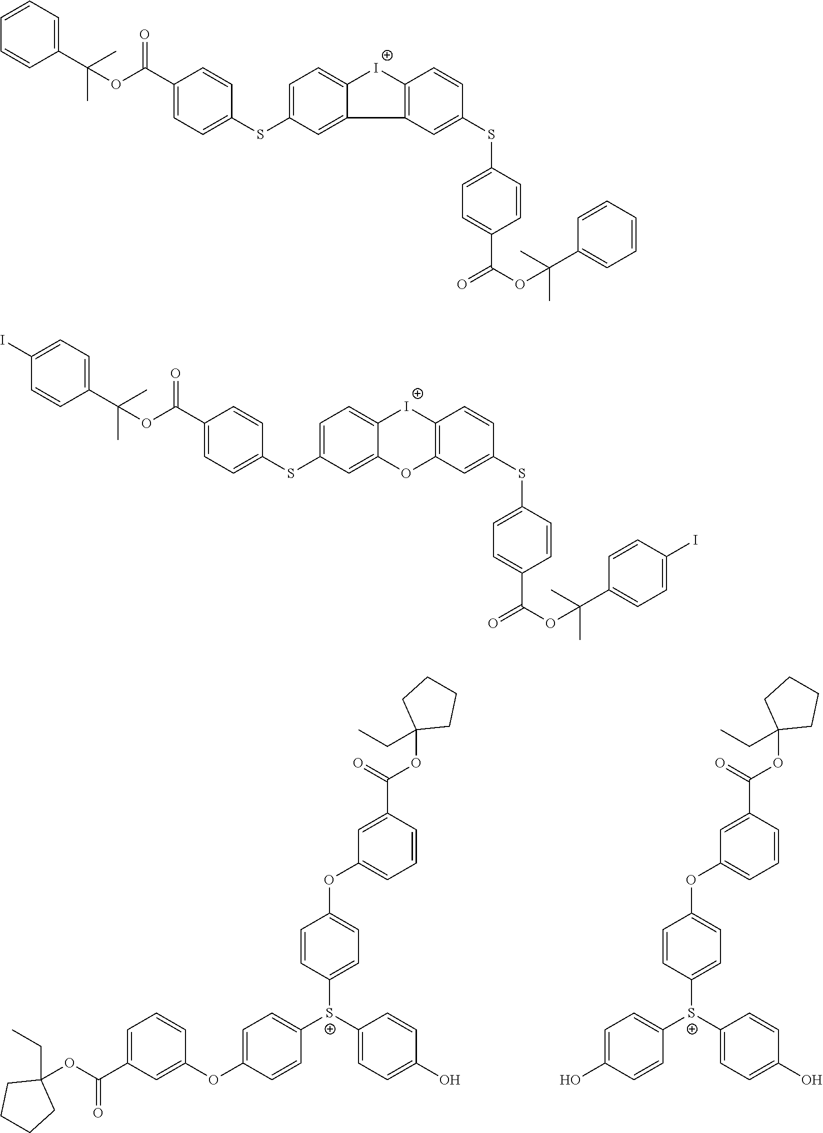 [SMILES: CC(C)(OC(=O)c1ccc(Sc2ccc3c(c2)-c2cc(Sc4ccc(C(=O)OC(C)(C)c5ccccc5)cc4)ccc2[I+]3)cc1)c1ccccc1 ; CC(C)(OC(=O)c1ccc(Sc2ccc3c(c2)Oc2cc(Sc4ccc(C(=O)OC(C)(C)c5ccc(I)cc5)cc4)ccc2[I+]3)cc1)c1ccc(I)cc1 ; CCC1(OC(=O)c2cccc(Oc3ccc([S+](c4ccc(O)cc4)c4ccc(Oc5cccc(C(=O)OC6(CC)CCCC6)c5)cc4)cc3)c2)CCCC1 ; CCC1(OC(=O)c2cccc(Oc3ccc([S+](c4ccc(O)cc4)c4ccc(O)cc4)cc3)c2)CCCC1]

In some aspects, the first component further includes an anion. Any suitable anion may be used. For example, in some embodiments, the first component may further include an anion selected from halide, hexafluorophosphate, or an anion group, wherein the anion group includes sulfonate, sulfonamidate, sulfonimidate, methide, borate, or carboxylate. It is to be understood that the anion group includes an organic group that is bonded to the stated anionic moiety. Exemplary organic groups include C₁₋₁₀₀ or C₁₋₆₀ organic groups that may contain one or more heteroatoms therein. The organic groups may be halogenated or non-halogenated. Typically, the organic groups may include one or more of —O—, —S—, —Te—, —Se—, —C(O)—, C(O)O—, —N(R′)—, —C(O)N(R′)—, —S(O)—, —S(O)₂—, —C(S)—, —C(Te)—, —C(Se)—, substituted or unsubstituted C₁₋₃₀ alkyl, substituted or unsubstituted C₃₋₃₀ cycloalkyl, substituted or unsubstituted C₃₋₃₀ heterocycloalkyl, substituted or unsubstituted C₆₋₃₀ aryl, substituted or unsubstituted C₃₋₃₀ heteroaryl, or a combination thereof, wherein each R′ is independently hydrogen, deuterium, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

Exemplary anion groups having a sulfonate group include one or more of the following:

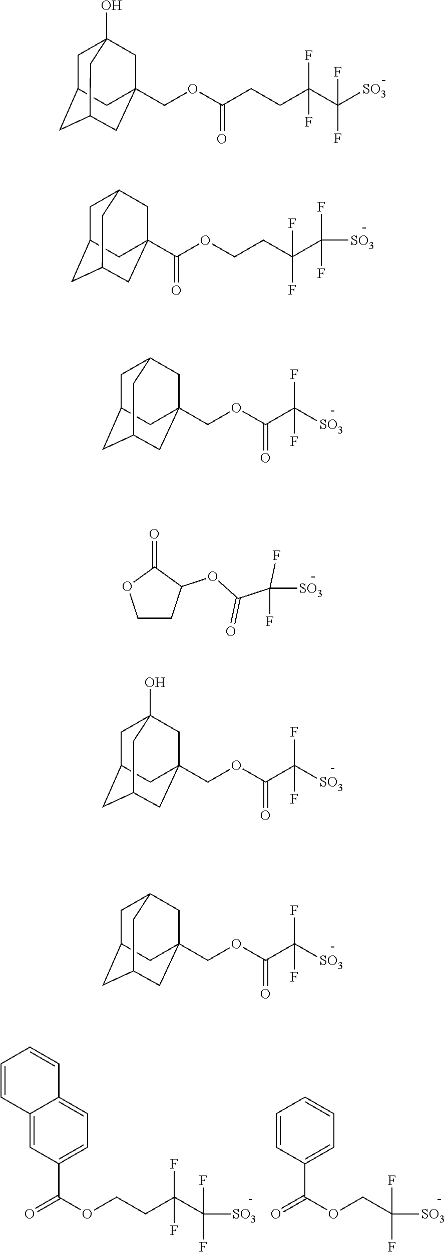

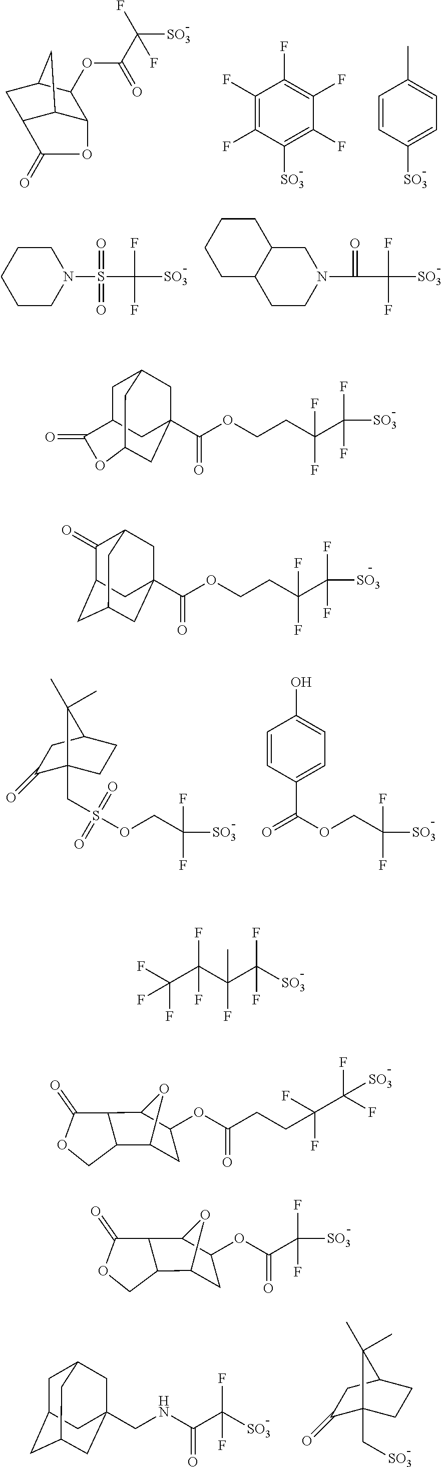

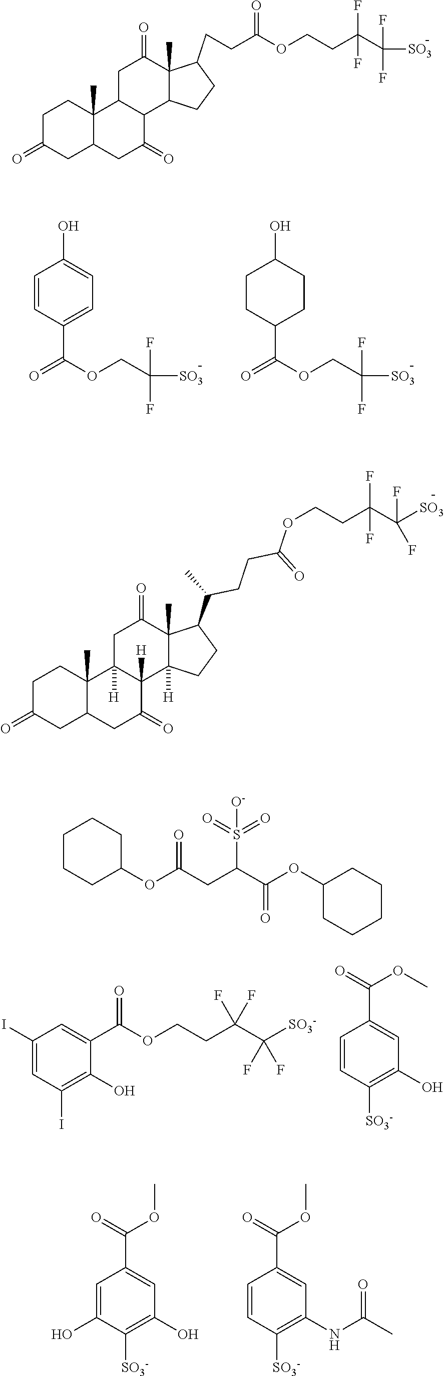

Exemplary non-sulfonated anion groups include one or more of the following:

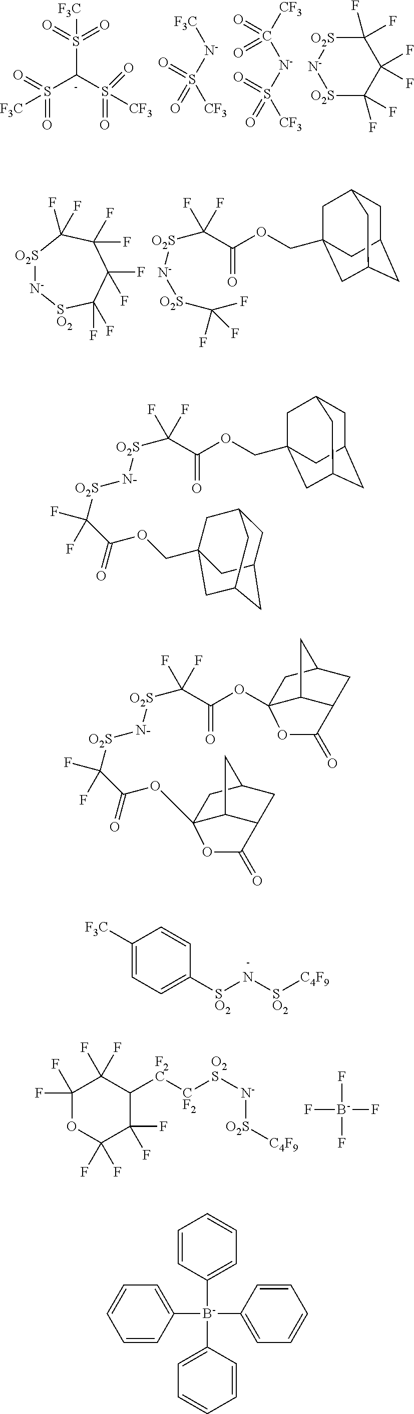

The photoacid generator onium salts of the present disclosure may be prepared by methods known in the art and as exemplified in the present examples that are disclosed in further detail below.

The photoresist composition also includes the second component, wherein the second component comprises a hydroxyaryl group, and the second component does not comprise an acid labile group.

The second component may be any suitable material that includes a hydroxyaryl group. The second component may be polymeric or non-polymeric.

In some embodiments, the second component may be a polymer comprising a repeating unit of Formula (3):

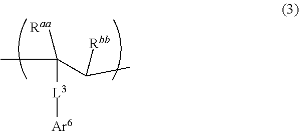

In Formula (3), R^{aa} is hydrogen, halogen, cyano, substituted or unsubstituted C₁₋₁₀ alkyl, or substituted or unsubstituted C₁₋₁₀ fluoroalkyl. Preferably, R^{aa} may be hydrogen, fluorine, or substituted or unsubstituted C₁₋₅ alkyl, typically hydrogen or methyl.

In Formula (3), R^{bb} is hydrogen, —C(O)— forming a ring with L³, or a single bond forming a ring with Ar⁶. Preferably, R^{bb} may be hydrogen.

In Formula (3), L³ is a single bond or a divalent linking group comprising one or more of substituted or unsubstituted C₁₋₃₀ alkylene, substituted or unsubstituted C₃₋₃₀ cycloalkylene, substituted or unsubstituted C₁₋₃₀ heterocycloalkylene, substituted or unsubstituted C₆₋₃₀ arylene, substituted or unsubstituted C₇₋₃₀ arylalkylene, substituted or unsubstituted C₁₋₃₀ heteroarylene, or substituted or unsubstituted C₃₋₃₀ heteroarylalkylene, —O—, —C(O)—, —C(O)—O—, —C(O)—N(R^{2a}), —S—, —S(O)₂—, —N(R^{2a})—S(O)₂—, —C₁₋₁₂ hydrocarbylene-, —O—(C₁₋₁₂ hydrocarbylene)-, —C(O)—O—(C₁₋₁₂ hydrocarbylene)-, or —C(O)—O—(C₁₋₁₂ hydrocarbylene)-O—, wherein R^{2a} is hydrogen, C₁₋₆ alkyl, or a single bond forming the ring with R^{bb}, provided that R^{bb} is —C(O)— forming the ring with L³ when R^{2a} is the single bond forming the ring with R^{bb}.

In Formula (3), Ar⁶ comprises a C₆₋₃₀ aryl group, a C₃₋₃₀ heteroaryl group, or a combination thereof, each comprising at least one hydroxy substituent, and each optionally further substituted with one or more non-hydrogen substituents. The C₆₋₃₀ aryl and the C₃₋₃₀ heteroaryl groups may each be monocyclic or polycyclic. It is to be understood that when the “monocyclic or polycyclic C₃₋₃₀ aryl group” is polycyclic, the number of carbon atoms is sufficient for the group to be chemically feasible. For example, the “monocyclic or polycyclic C₆₋₃₀ aryl” may refer to “a monocyclic C₆ aryl group or a polycyclic C₁₀₋₃₀ aryl group”. Similarly, when the “monocyclic or polycyclic C₃₋₃₀ heteroaryl” is polycyclic, the number of carbon atoms is sufficient for the group to be chemically feasible. For example, the “monocyclic or polycyclic C₃₋₃₀ heteroaryl” may refer to “a monocyclic C₃₋₆ heteroaryl or a polycyclic C₅₋₃₀ heteroaryl”. Exemplary groups for Ar⁶ include, but are not limited to, benzene, naphthalene, anthracene, phenanthrene, pyrene, coronene, triphenylene, chrysene, phenalene, benz[a]anthracene, dibenz[a,h]anthracene, or benzo[a]pyrene, each of which may be substituted or unsubstituted.

When the C₆₋₃₀ aryl and the C₃₋₃₀ heteroaryl groups are polycyclic, the ring or ring groups can be fused (such as naphthyl or the like), directly linked (such as biaryls, biphenyl, or the like), and/or bridged by a heteroatom (such as triphenylamino or diphenylene ether).

In Formula (3), Ar⁶ is substituted with at least one hydroxy group. In some embodiments, Ar⁶ may be substituted with two or more hydroxy groups, for example, 1 to 12 hydroxy groups, preferably from 1 to 6 hydroxy groups, and typically from 1 to 3 hydroxy groups.

Non-limiting examples of such repeating units of Formula (3) may include:

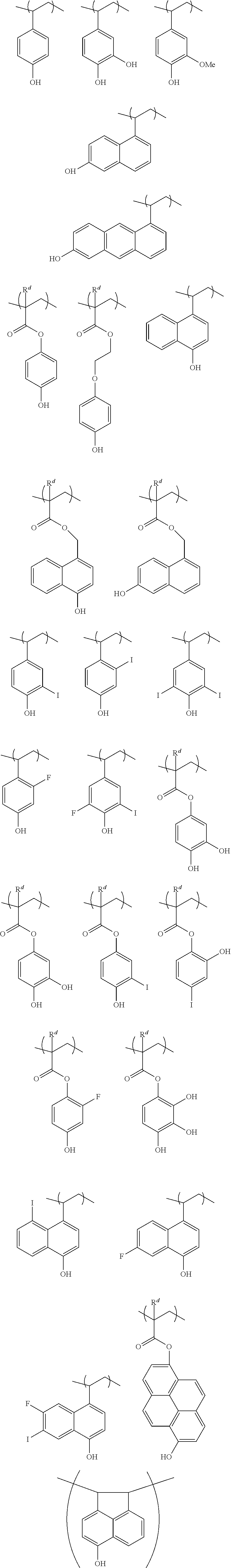

wherein R^{d} is hydrogen, halogen, substituted or unsubstituted C₁₋₆ alkyl, or substituted or unsubstituted C₃₋₆ cycloalkyl.

When the second component is a polymer comprising a repeating unit of Formula (3), the repeating unit including the hydroxyaryl group typically may be present in an amount from 10 to 90 mole percent (mol %), more typically from 15 to 75 mol %, and still more typically from 25 to 70 mol %, based on total repeating units of the polymer.

In some aspects, the polymer may further optionally include one or more additional repeating units. The additional repeating units may be, for example, one or more additional units for purposes of adjusting properties of the polymer. Exemplary additional repeating units may include those derived from one or more of (meth)acrylate, vinyl aromatic, vinyl ether, vinyl ketone, and/or vinyl ester monomers. The one or more additional repeating units, if present in the polymer, may be used in an amount of up to 80 mol %, typically from 3 to 50 mol %, based on total repeating units of the polymer.

In some embodiments, the second component may be a compound of Formula (4):

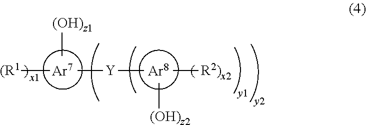

In Formula (4), Ar⁷ and each Ar⁸ are independently C₆-30 arylene, or C₃₋₃₀ heteroarylene. The C₆₋₃₀ aryl and the C₃₋₃₀ heteroaryl groups may each be monocyclic or polycyclic. It is to be understood that when the “monocyclic or polycyclic C₃₋₃₀ aryl group” is polycyclic, the number of carbon atoms is sufficient for the group to be chemically feasible. For example, the “monocyclic or polycyclic C₆₋₃₀ aryl” may refer to “a monocyclic C₆ aryl group or a polycyclic C₁₀₋₃₀ aryl group”. Similarly, when the “monocyclic or polycyclic C₃₋₃₀ heteroaryl” is polycyclic, the number of carbon atoms is sufficient for the group to be chemically feasible. For example, the “monocyclic or polycyclic C₃₋₃₀ heteroaryl” may refer to “a monocyclic C₃₋₆ heteroaryl or a polycyclic C₅₋₃₀ heteroaryl”. Exemplary groups for Ar⁷ to Ar⁸ include, but are not limited to, benzene, naphthalene, anthracene, phenanthrene, pyrene, coronene, triphenylene, chrysene, phenalene, benz[a]anthracene, dibenz[a,h]anthracene, or benzo[a]pyrene, each of which may be substituted or unsubstituted.

When the C₆₋₃₀ aryl and the C₃₋₃₀ heteroaryl groups are polycyclic, the ring or ring groups can be fused (such as naphthyl or the like), directly linked (such as biaryls, biphenyl, or the like), and/or bridged by a heteroatom (such as triphenylamino or diphenylene ether).

In Formula (4), R¹ and R² are each independently a non-hydrogen substituent. For example, R¹ and R² may each independently be halogen, deuterium, substituted or unsubstituted C₁₋₂₀ alkyl, substituted or unsubstituted C₃₋₂₀ cycloalkyl, substituted or unsubstituted C₁₋₂₀ heterocycloalkyl, substituted or unsubstituted C₂₋₂₀ alkenyl, substituted or unsubstituted C₃₋₂₀ cycloalkenyl, substituted or unsubstituted C₃₋₂₀ heterocycloalkenyl, substituted or unsubstituted C₆₋₂₀ aryl, or substituted or unsubstituted C₂₋₂₀ heteroaryl.

Each of R¹ and R² may further include one or more divalent linking groups as a part of its structure. Exemplary divalent linking groups include, but are not limited to, one or more of —O—, —S—, —C(O)—, —C(O)O—, —N(R′)—, —C(O)N(R′)—, —S(O)—, —S(O)₂—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formula (4), each Y is independently a single bond, or a multivalent linking group, wherein the multivalent linking group is substituted or unsubstituted. As defined herein, a multivalent linking group is a linking group having a valency of two or greater (e.g., divalent, trivalent, tetravalent, etc.). Exemplary multivalent linking groups include, but are not limited to, —O—, —C(O)—, —C(O)O—, —N(R^{c})—, —C(O)N(R^{c})—, substituted or unsubstituted C₁₋₁₀ alkylene, substituted or unsubstituted C₃₋₁₀ cycloalkylene, substituted or unsubstituted C₃₋₁₀ heterocycloalkylene, substituted or unsubstituted C₆₋₁₀ arylene, or substituted or unsubstituted C₃₋₁₀ heteroarylene.

In Formula (4), x1 and x2 are each independently an integer from 0 to 10. Typically, x1 and x2 are each independently 0, 1, 2, or 3.

In Formula (4), y1 is an integer from 1 to 5. Typically, y1 is an integer from 1 to 3.

In Formula (4), y2 is an integer from 0 to 10. Typically, Y is 1.

In Formula (4), z1 and z2 are each independently an integer from 0 to 10, provided z1+z2 is at least 2. Typically, z1 is 1, and each z2 is 1.

Non-limiting examples of compounds of Formula (4) may include:

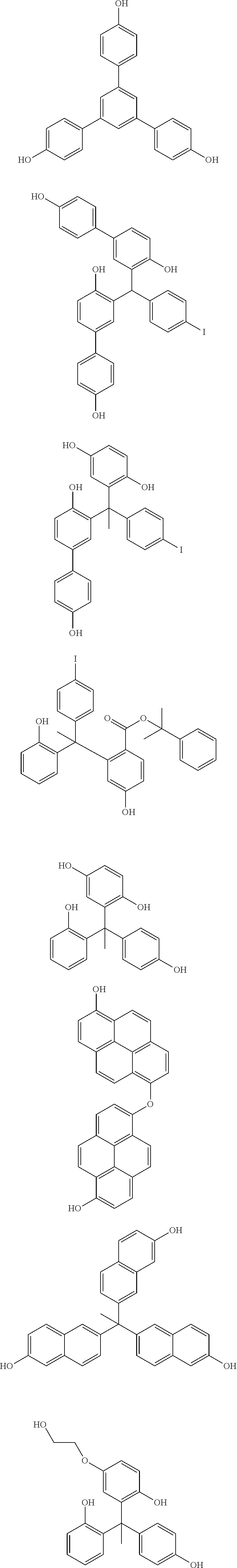

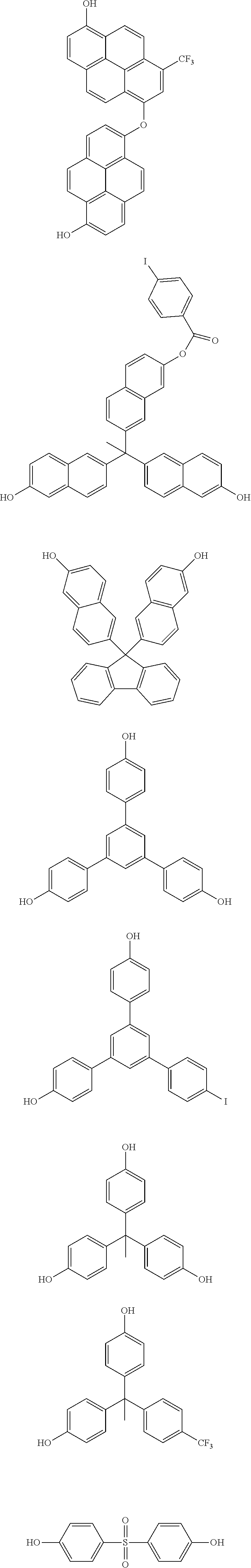

In some embodiments, the second component may be a condensation polymer derived from the monomer of Formula (5):

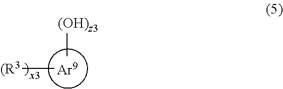

In Formula (5), Ar⁹ is C₆₋₃₀ aryl or C₃₋₃₀ heteroaryl. The C₆₋₃₀ aryl and the C₃₋₃₀ heteroaryl groups may each be monocyclic or polycyclic, as described above for Ar¹ to Ar⁵. Exemplary groups for Ar⁹ include, but are not limited to, benzene, naphthalene, anthracene, phenanthrene, pyrene, coronene, triphenylene, chrysene, phenalene, benz[a]anthracene, dibenz[a,h]anthracene, or benzo[a]pyrene, each of which may be substituted or unsubstituted.

When the C₆₋₃₀ aryl and the C₃₋₃₀ heteroaryl groups are polycyclic, the ring or ring groups can be fused (such as naphthyl or the like), directly linked (such as biaryls, biphenyl, or the like), and/or bridged by a heteroatom (such as triphenylamino or diphenylene ether).

In Formula (5), R³ is a non-hydrogen substituent. For example, each R³ may be independently deuterium, halogen, substituted or unsubstituted C₁₋₃₀ alkyl, substituted or unsubstituted C₃₋₃₀ cycloalkyl, substituted or unsubstituted C₃₋₃₀ cycloalkene, substituted or unsubstituted C₃₋₃₀ heterocycloalkyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₆₋₃₀ aryloxy, substituted or unsubstituted C₃₋₃₀ heteroaryl, substituted or unsubstituted C₄₋₃₀ alkylheteroaryl, substituted or unsubstituted C₄₋₃₀ heteroarylalkyl, or substituted or unsubstituted C₃₋₃₀ heteroaryloxy.

Each R³ may include one or more divalent linking groups as part of its structure. Exemplary divalent linking groups include, but are not limited to, one or more of —O—, —S—, —C(O)—, —C(O)O—, —N(R′)—, —C(O)N(R′)—, —S(O)—, —S(O)₂—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formula (5), x3 is an integer from 0 to 5. Typically, x3 is 0, 1, 2, or 3.

In Formula (5), z3 is an integer from 1 to 10. Typically, z3 is 1.

In an embodiment, the monomer of Formula (5) may be represented by Formula (5a):

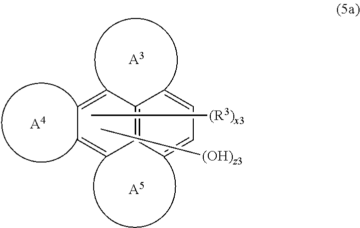

wherein A³, A⁴, and A⁵ each may be present or absent, and each independently represents 1 to 3 fused aromatic rings.

In Formula (5a), R³, x3, and z3 are each as defined for Formula (5).

In an embodiment, the monomer of Formula (5) may be represented by one of Formulae (5b) to (5d):

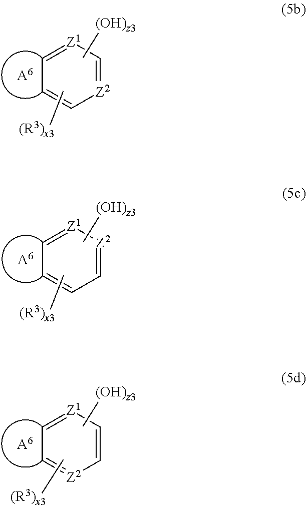

wherein A⁶ may be present or absent and represents 1 to 3 fused aromatic rings. It is preferred that A⁶ represents 1 to 3 aromatic rings, and more preferably 1 to 2 fused aromatic rings, and most preferably 1 fused aromatic ring.

In Formulae (5b) to (5d), R³, x3, and z3 are each as defined for Formula (5).

In an embodiment, the monomer of Formula (5) may be represented by Formula (5e):

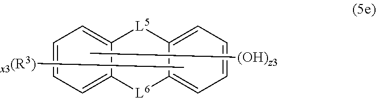

In Formula (5e), L⁵ is a single bond, —O—, —S—, —S(O)—, —SO₂—, —C(O)—, —CR⁴¹R⁴²—, —NR⁴³—, or —PR⁴⁴—, wherein R⁴¹ to R⁴⁴ are each independently hydrogen, deuterium, substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₂₋₃₀ heterocycloalkyl, substituted or unsubstituted C₆₋₃₀ aryl, substituted or unsubstituted C₇₋₃₀ arylalkyl, substituted or unsubstituted C₃₋₃₀ heteroaryl, or substituted or unsubstituted C₄₋₃₀ heteroarylalkyl. Preferably, L⁵ is —O— or —NR⁴³—, more preferably —NR⁴³—.

In Formula (5e), L⁶ is absent, a single bond, —O—, —S—, —S(O)—, —SO₂—, —C(O)—, substituted or unsubstituted C₁₋₂ alkylene, substituted or unsubstituted C₆₋₃₀ arylene, or substituted or unsubstituted C₅₋₃₀ heteroarylene. Preferably, L⁶ is a single bond.

In Formula (5e), R³, x3, and z3 are each as defined for Formula (5).

Non-limiting examples of compounds of Formula (5) may include:

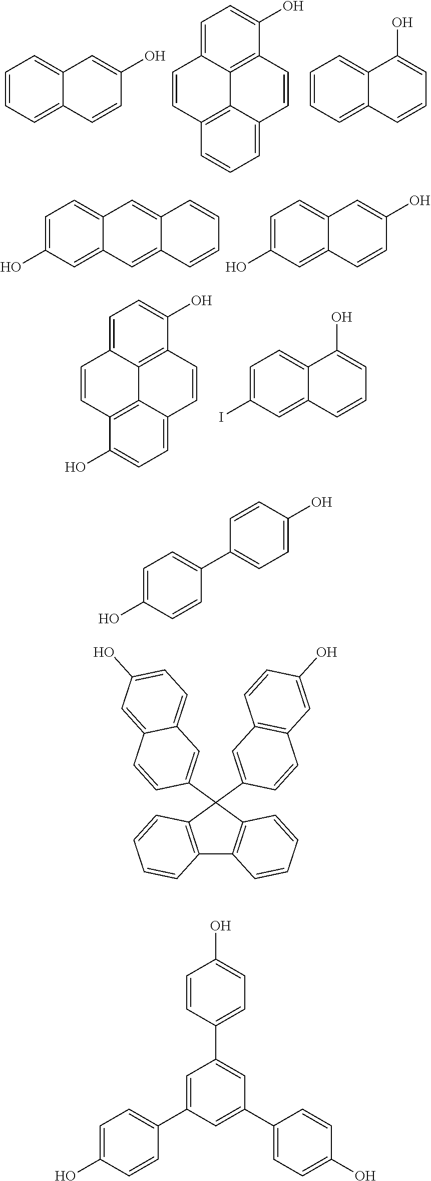

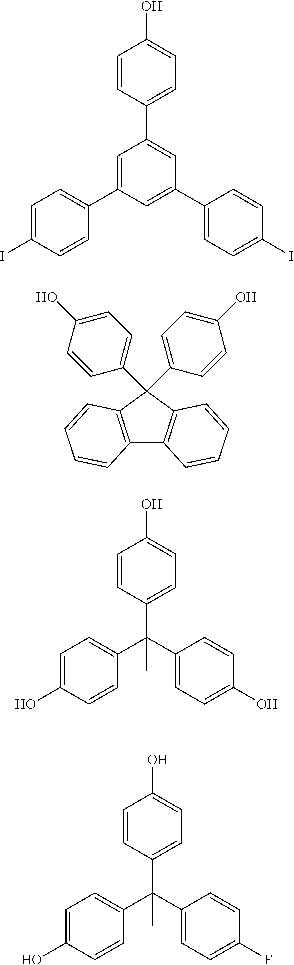

As used herein, “condensation polymers” are the product of condensation polymerization of one or more hydroxy-containing aromatic compounds with another monomer selected from aliphatic or aromatic carbonyl compounds, benzyl ethers, benzyl alcohols, or benzyl halides. Typically, the condensation polymers are the product of polycondensation of an aromatic compound of Formula (5) with formaldehyde-type or aromatic aldehyde co-monomers. For example, the condensation polymer may include a repeating unit derived from the reaction of the compound of Formula (5) with an aldehyde of the formula Ar′—CHO or a ketone of the formula Ar′—CO—Ar′, wherein each Ar′ is independently a substituted or unsubstituted C₆₋₃₀ aromatic group.

A variety of acids may suitably be used as catalysts in the preparation of the condensation polymer derived from the monomer of Formula (5). Exemplary acids include, without limitation, organic carboxylic acids and dicarboxylic acids such as propionic acid and oxalic acid, mineral acids and sulfonic acids, and preferably the acid catalyst is a mineral acid or a sulfonic acid. Suitable mineral acids are HF, HCl, HBr, HNO₃, H₂SO₄, H₃PO₄, and HClO₄. Suitable sulfonic acids include alkane sulfonic acids and aryl sulfonic acids, such as methanesulfonic acid, ethane sulfonic acid, propane sulfonic acid, phenyl sulfonic acid, phenol sulfonic acid, para-toluenesulfonic acid, and cresol sulfonic acid. Preferred acid catalysts are HCl, HBr, HNO₃, H₂SO₄, H₃PO₄, methane sulfonic acid, ethane sulfonic acid, phenyl sulfonic acid, phenol sulfonic acid, and para-toluenesulfonic acid (pTSA).

When the second component is a polymer, the polymer typically has a weight average molecular weight (M_{w}) of 500 to 20000 Dalton (Da), preferably from 500 to 15000 Da, and more preferably from 500 to 10000 Da, as determined by gel permeation chromatography (GPC) using polystyrene standards.

The photoresist composition further includes a solvent. Suitable solvents include, for example: aliphatic hydrocarbons such as hexane and heptane; aromatic hydrocarbons such as toluene and xylene; halogenated hydrocarbons such as dichloromethane, 1,2-dichloroethane and 1-chlorohexane; alcohols such as methanol, ethanol, 1-propanol, iso-propanol, tert-butanol, 2-methyl-2-butanol, 4-methyl-2-pentanol, and diacetone alcohol (4-hydroxy-4-methyl-2-pentanone) (DAA); propylene glycol monomethyl ether (PGME); ethers such as diethyl ether, tetrahydrofuran, 1,4-dioxane, and anisole; ketones such as acetone, methyl ethyl ketone, methyl iso-butyl ketone, 2-heptanone, and cyclohexanone (CHO); esters such as ethyl acetate, n-butyl acetate, propylene glycol monomethyl ether acetate (PGMEA), ethyl lactate (EL), hydroxyisobutyrate methyl ester (HBM), and ethyl acetoacetate; lactones such as gamma-butyrolactone (GBL) and epsilon-caprolactone; lactams such as N-methyl pyrrolidone; nitriles such as acetonitrile and propionitrile; cyclic or non-cyclic carbonate esters such as propylene carbonate, dimethyl carbonate, ethylene carbonate, propylene carbonate, diphenyl carbonate, and propylene carbonate; polar aprotic solvents such as dimethyl sulfoxide and dimethyl formamide; water; and combinations thereof. Of these, preferred solvents are PGME, PGMEA, EL, GBL, HBM, CHO, DAA, and combinations thereof.

The total solvent content (i.e., cumulative solvent content for all solvents) in the photoresist compositions is typically from 40 to 99 wt %, for example, from 60 to 99 wt %, or from 85 to 99 wt %, based on total solids of the photoresist composition. The desired solvent content will depend, for example, on the desired thickness of the coated photoresist layer and coating conditions.

In the photoresist compositions of the invention, the first component is typically present in the photoresist composition in an amount from 50 to 99.9 wt %, typically from 50 to 99 wt %, and more typically from 50 to 95 wt %, based on total solids of the photoresist composition. In still other embodiments, the first component may be present in the photoresist composition in an amount from 80 to 99.9 wt %, or from 85 to 99 wt %, or from 90 to 99 wt %, based on total solids of the photoresist composition. It will be understood that total solids includes the first component, the second component, and other non-solvent components.

The photoresist composition may further include an additional photoacid generator that is different from the salts of Formula (1) and (2). The additional PAG may be in ionic or non-ionic form. The additional PAG may be in polymeric or non-polymeric form. In polymeric form, the additional PAG may be present as a moiety in a repeating unit of a polymer that is derived from a polymerizable PAG monomer.

Suitable additional PAG compounds may be of the formula G⁺A⁻, wherein G⁺ is a photoactive cation and A⁻ is an anion that can generate a photoacid. The photoactive cation is preferably chosen from onium cations, preferably iodonium or sulfonium cations. Particularly suitable anions include those whose conjugated acids have a pKa of from −15 to 0, or from −14 to 0, or from −13 to 0. The anion is typically an organic anion having a sulfonate group or a non-sulfonate-type group, such as carboxylate, sulfonamidate, sulfonimidate, methide, arsenate, or borate.

Exemplary onium salts may include, for example, triphenylsulfonium trifluoromethanesulfonate, (p-tert-butoxyphenyl)diphenylsulfonium trifluoromethanesulfonate, tris(p-tert-butoxyphenyl)sulfonium trifluoromethanesulfonate, triphenylsulfonium p-toluenesulfonate; di-t-butyphenyliodonium perfluorobutanesulfonate, and di-t-butyphenyliodonium camphorsulfonate. Other useful additional PAG compounds are known in the art of chemically amplified photoresists and include, for example: non-ionic sulfonyl compounds, for example, 2-nitrobenzyl-p-toluenesulfonate, 2,6-dinitrobenzyl-p-toluenesulfonate, and 2,4-dinitrobenzyl-p-toluenesulfonate; sulfonic acid esters, for example, 1,2,3-tris(methanesulfonyloxy)benzene, 1,2,3-tris(trifluoromethanesulfonyloxy)benzene, and 1,2,3-tris(p-toluenesulfonyloxy)benzene; diazomethane derivatives, for example, bis(benzenesulfonyl)diazomethane, bis(p-toluenesulfonyl)diazomethane; glyoxime derivatives, for example, bis-O-(p-toluenesulfonyl)-α-dimethylglyoxime, and bis-O-(n-butanesulfonyl)-α-dimethylglyoxime; sulfonic acid ester derivatives of an N-hydroxyimide compound, for example, N-hydroxysuccinimide methanesulfonic acid ester, N-hydroxysuccinimide trifluoromethanesulfonic acid ester; and halogen-containing triazine compounds, for example, 2-(4-methoxyphenyl)-4,6-bis(trichloromethyl)-1,3,5-triazine, and 2-(4-methoxynaphthyl)-4,6-bis(trichloromethyl)-1,3,5-triazine. Suitable additional PAGs are further described in U.S. Pat. Nos. 8,431,325 and 4,189,323.

Typically, when the photoresist composition includes an additional PAG, the additional PAG is present in the photoresist composition in an amount of from 0.1 to 55 wt %, more typically 1 to 25 wt %, based on total solids of the photoresist composition. When present in polymeric form, the additional PAG is typically included in a polymer in an amount from 1 to 25 mol %, more typically from 1 to 8 mol %, or from 2 to 6 mol %, based on total repeating units in the polymer.

In some aspects, the photoresist composition may further include a material that comprises one or more base-labile groups (a “base-labile material”). As referred to herein, base-labile groups are functional groups that can undergo cleavage reaction to provide polar groups such as hydroxyl, carboxylic acid, sulfonic acid, and the like, in the presence of an aqueous alkaline developer after exposure and post-exposure baking steps. The base-labile group will not react significantly (e.g., will not undergo a bond-breaking reaction) prior to a development step of the photoresist composition that comprises the base-labile group. Thus, for instance, a base-labile group will be substantially inert during pre-exposure soft-bake, exposure, and post-exposure bake steps. By “substantially inert” it is meant that ≤5%, typically ≤1%, of the base-labile groups (or moieties) will decompose, cleave, or react during the pre-exposure soft-bake, exposure, and post-exposure bake steps. The base-labile group is reactive under typical photoresist development conditions using, for example, an aqueous alkaline photoresist developer such as a 0.26 normal (N) aqueous solution of tetramethylammonium hydroxide (TMAH). For example, a 0.26 N aqueous solution of TMAH may be used for single puddle development or dynamic development, e.g., where the 0.26 N TMAH developer is dispensed onto an imaged photoresist layer for a suitable time such as 10 to 120 seconds(s). An exemplary base-labile group is an ester group, typically a fluorinated ester group. When coated on a substrate, the base-labile material can segregate from other solid components of the photoresist composition to a top surface of the formed photoresist layer.

In some aspects, the base-labile material may be a polymeric material, also referred to herein as a base-labile polymer, which may include one or more repeating units comprising one or more base-labile groups. For example, the base-labile polymer may comprise a repeating unit comprising 2 or more base-labile groups that are the same or different. A preferred base-labile polymer includes at least one repeating unit comprising 2 or more base-labile groups, for example a repeating unit comprising 2 or 3 base-labile groups.

The base-labile polymer may be prepared using any suitable methods in the art, including those described herein for the first and second polymers. For example, the base-labile polymer may be obtained by polymerization of the respective monomers under any suitable conditions, such as by heating at an effective temperature, irradiation with actinic radiation at an effective wavelength, or a combination thereof. Additionally, or alternatively, one or more base-labile groups may be grafted onto the backbone of a polymer using suitable methods.

In some aspects, the base-labile material is a single molecule comprising one more base-labile ester groups, preferably one or more fluorinated ester groups. The base-labile materials that are single molecules typically have a M_{w} in the range from 50 to 1,500 Da.

When present, the base-labile material is typically present in the photoresist compositions in an amount of from 0.01 to 10 wt % or 2 to 7 w %, typically from 1 to 5 wt %, based on total solids of the photoresist composition.

The photoresist composition may further include one or more additional, optional additives. For example, optional additives may include actinic and contrast dyes, anti-striation agents, plasticizers, speed enhancers, sensitizers, photo-decomposable quenchers (PDQ) (and, also known as photo-decomposable bases), basic quenchers, thermal acid generators, surfactants, and the like, or combinations thereof. If present, the optional additives are typically present in the photoresist compositions in an amount of from 0.01 to 10 wt %, based on total solids of the photoresist composition.

PDQs generate a weak acid upon irradiation. The acid generated from a photo-decomposable quencher is not strong enough to react rapidly with acid-labile groups that are present in the resist matrix. Exemplary photo-decomposable quenchers include, for example, photo-decomposable cations, and preferably those also useful for preparing strong acid generator compounds, paired with an anion of a weak acid (pKa>−2) such as, for example, an anion of a C₁₋₂₀ carboxylic acid or C₁₋₂₀ sulfonic acid. Exemplary carboxylic acids include formic acid, acetic acid, propionic acid, tartaric acid, succinic acid, cyclohexanecarboxylic acid, benzoic acid, salicylic acid, and the like. Exemplary sulfonic acids include p-toluene sulfonic acid, camphor sulfonic acid and the like. In a preferred embodiment, the photo-decomposable quencher is a photo-decomposable organic zwitterion compound such as diphenyliodonium-2-carboxylate.

The photo-decomposable quencher may be in non-polymeric or polymer-bound form. When in polymeric form, the photo-decomposable quencher is present in polymerized units on the first polymer or second polymer. The polymerized units containing the photo-decomposable quencher are typically present in an amount from 0.1 to 30 mole %, preferably from 1 to 10 mole % and more preferably from 1 to 2 mole %, based on total repeating units of the polymer.

Exemplary PDQs include, but are not limited to, the following compounds and derivatives thereof:

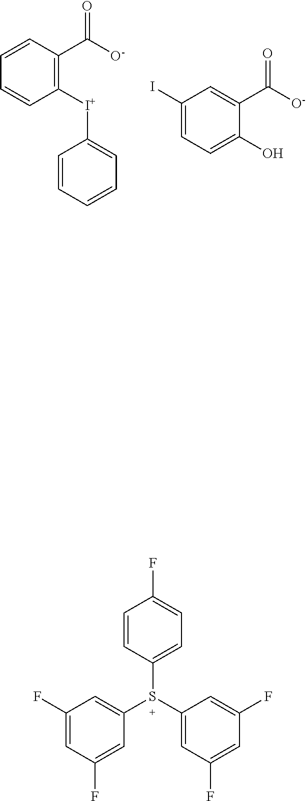

Exemplary basic quenchers include, for example, linear aliphatic amines such as tributylamine, trioctylamine, triisopropanolamine, tetrakis(2-hydroxypropyl)ethylenediamine: n-tert-butyldiethanolamine, tris(2-acetoxy-ethyl)amine, 2,2′,2″,2′″-(ethane-1,2-diylbis(azanetriyl))tetraethanol, 2-(dibutylamino)ethanol, and 2,2′,2″-nitrilotriethanol; cyclic aliphatic amines such as 1-(tert-butoxycarbonyl)-4-hydroxypiperidine, tert-butyl 1-pyrrolidinecarboxylate, tert-butyl 2-ethyl-1H-imidazole-1-carboxylate, di-tert-butyl piperazine-1,4-dicarboxylate, and N-(2-acetoxy-ethyl)morpholine; aromatic amines such as pyridine, di-tert-butyl pyridine, and pyridinium; linear and cyclic amides and derivatives thereof such as N,N-bis(2-hydroxyethyl) pivalamide, N,N-diethylacetamide, N1,N1,N3,N3-tetrabutylmalonamide, 1-methylazepan-2-one, 1-allylazepan-2-one, and tert-butyl 1,3-dihydroxy-2-(hydroxymethyl) propan-2-ylcarbamate; ammonium salts such as quaternary ammonium salts of sulfonates, sulfamates, carboxylates, and phosphonates; imines such as primary and secondary aldimines and ketimines; diazines such as optionally substituted pyrazine, piperazine, and phenazine; diazoles such as optionally substituted pyrazole, thiadiazole, and imidazole; and optionally substituted pyrrolidones such as 2-pyrrolidone and cyclohexyl pyrrolidine.

The basic quenchers may be in non-polymeric or polymer-bound form. When in polymeric form, the quencher may be present in repeating units of the polymer. The repeating units containing the quencher are typically present in an amount of from 0.1 to 30 mole %, preferably from 1 to 10 mole % and more preferably from 1 to 2 mole %, based on total repeating units of the polymer.

Exemplary surfactants include fluorinated and non-fluorinated surfactants and can be ionic or non-ionic, with non-ionic surfactants being preferable. Exemplary fluorinated non-ionic surfactants include perfluoro C₄ surfactants such as FC-4430 and FC-4432 surfactants, available from 3M Corporation; and fluorodiols such as POLYFOX PF-636, PF-6320, PF-656, and PF-6520 fluorosurfactants from Omnova. In an aspect, the photoresist composition further includes a surfactant polymer including a fluorine-containing repeating unit.

Patterning methods using the photoresist compositions of the invention will now be described. Suitable substrates on which the photoresist compositions can be coated include electronic device substrates. A wide variety of electronic device substrates may be used in the present invention, such as: semiconductor wafers; polycrystalline silicon substrates; packaging substrates such as multichip modules; flat panel display substrates; substrates for light emitting diodes (LEDs) including organic light emitting diodes (OLEDs); and the like, with semiconductor wafers being typical. Such substrates are typically composed of one or more of silicon, polysilicon, silicon oxide, silicon nitride, silicon oxynitride, silicon germanium, gallium arsenide, aluminum, sapphire, tungsten, titanium, titanium-tungsten, nickel, copper, and gold. Suitable substrates may be in the form of wafers such as those used in the manufacture of integrated circuits, optical sensors, flat panel displays, integrated optical circuits, and LEDs. Such substrates may be any suitable size. Typical wafer substrate diameters are 200 to 300 millimeters (mm), although wafers having smaller and larger diameters may be suitably employed. The substrates may include one or more layers or structures which may optionally include active or operable portions of devices being formed.

Typically, one or more lithographic layers such as a hardmask layer, for example, a spin-on-carbon (SOC), amorphous carbon, or metal hardmask layer, a CVD layer such as a silicon nitride (SiN), a silicon oxide (SiO), or silicon oxynitride (SiON) layer, an organic or inorganic underlayer, or combinations thereof, are provided on an upper surface of the substrate prior to coating a photoresist composition. Such layers, together with an overcoated photoresist layer, form a lithographic material stack.

Optionally, a layer of an adhesion promoter may be applied to the substrate surface prior to coating the photoresist compositions. If an adhesion promoter is desired, any suitable adhesion promoter for polymer films may be used, such as silanes, typically organosilanes such as trimethoxyvinylsilane, triethoxyvinylsilane, hexamethyldisilazane, or an aminosilane coupler such as gamma-aminopropyltriethoxysilane. Particularly suitable adhesion promoters include those sold under the AP™ 3000, AP™ 8000, and AP™ 9000S designations, available from DuPont Electronic Materials International (Marlborough, Massachusetts).

The photoresist composition may be coated on the substrate by any suitable method, including spin coating, spray coating, dip coating, doctor blading, or the like. For example, applying the layer of photoresist may be accomplished by spin coating the photoresist in solvent using a coating track, in which the photoresist is dispensed on a spinning wafer. During dispensing, the wafer is typically spun at a speed of up to 4,000 rotations per minute (rpm), for example, from 200 to 3,000 rpm, for example, from 1,000 to 2,500 rpm, for a period from 15 to 120 seconds to obtain a layer of the photoresist composition on the substrate. It will be appreciated by those skilled in the art that the thickness of the coated layer may be adjusted by changing the spin speed and/or the total solids of the composition. A photoresist composition layer formed from the compositions of the invention typically has a dried layer thickness from 1 nanometer (nm) to 120 micrometers (μm), preferably from greater than 5 nm to 110 μm, and more preferably from 6 to 100 μm. In some embodiments, the photoresist composition layer formed from the compositions may have a dried layer thickness from 10 nm to 5 μm, or from 3 to 20 μm.

The photoresist composition is typically next soft-baked to minimize the solvent content in the layer, thereby forming a tack-free coating and improving adhesion of the layer to the substrate. The soft bake is performed, for example, on a hotplate or in an oven, with a hotplate being typical. The soft bake temperature and time will depend, for example, on the photoresist composition and thickness. The soft bake temperature is typically from 80 to 170° C., and more typically from 90 to 150° C. The soft bake time is typically from 10 seconds to 20 minutes, more typically from 1 to 10 minutes, and still more typically from 1 to 2 minutes. The heating time can be readily determined by one of ordinary skill in the art based on the ingredients of the composition.

The photoresist layer is next pattern-wise exposed to activating radiation to create a difference in solubility between exposed and unexposed regions. Reference herein to exposing a photoresist composition to radiation that is activating for the composition indicates that the radiation can form a latent image in the photoresist composition. The exposure is typically conducted through a patterned photomask that has optically transparent and optically opaque regions corresponding to regions of the resist layer to be exposed and unexposed, respectively. Such exposure may, alternatively, be conducted without a photomask in a direct writing method, typically used for e-beam lithography. The activating radiation typically has a wavelength of sub-400 nm, sub-300 nm or sub-200 nm, with 248 nm (KrF), 193 nm (ArF), 13.5 nm (EUV) wavelengths or e-beam lithography being preferred. The methods find use in immersion or dry (non-immersion) lithography techniques. The exposure energy is typically from 1 to 200 millijoules per square centimeter (mJ/cm²), preferably from 10 to 100 mJ/cm² and more preferably from 20 to 50 mJ/cm², dependent upon the exposure tool and components of the photoresist composition.

Following exposure of the photoresist layer, a post-exposure bake (PEB) of the exposed photoresist layer may be performed. The PEB can be conducted, for example, on a hotplate or in an oven, with a hotplate being typical. Conditions for the PEB will depend, for example, on the photoresist composition and layer thickness. The PEB is typically conducted at a temperature from 70 to 150° C., preferably from 75 to 120° C., and a time from 30 to 120 seconds. A latent image defined by the polarity-switched (exposed regions) and unswitched regions (unexposed regions) is formed in the photoresist.

The exposed photoresist layer is then developed with a suitable developer to selectively remove those regions of the layer that are soluble in the developer while the remaining insoluble regions form the resulting photoresist pattern relief image. In the case of a positive-tone development (PTD) process, the exposed regions of the photoresist layer are removed during development and unexposed regions remain. Conversely, in a negative-tone development (NTD) process, the exposed regions of the photoresist layer remain, and unexposed regions are removed during development. Application of the developer may be accomplished by any suitable method such as described above with respect to application of the photoresist composition, with spin coating being typical. The development time is for a period effective to remove the soluble regions of the photoresist, with a time of from 5 to 60 seconds being typical. Development is typically conducted at room temperature.

Suitable developers for a PTD process include aqueous base developers, for example, quaternary ammonium hydroxide solutions such as tetramethylammonium hydroxide (TMAH), preferably 0.26 normal (N) TMAH, tetraethylammonium hydroxide, tetrabutylammonium hydroxide, sodium hydroxide, potassium hydroxide, sodium carbonate, potassium carbonate, and the like. Suitable developers for an NTD process are organic solvent-based, meaning the cumulative content of organic solvents in the developer is 50 wt % or more, typically 95 wt % or more, 98 wt % or more, or 100 wt %, based on total weight of the developer. Suitable organic solvents for the NTD developer include, for example, those chosen from ketones, esters, ethers, hydrocarbons, and mixtures thereof. The developer is typically 2-heptanone or n-butyl acetate.

A coated substrate may be formed from the photoresist compositions of the invention. Such a coated substrate includes: (a) a substrate having one or more layers to be patterned on a surface thereof; and (b) a layer of the photoresist composition over the one or more layers to be patterned.

The photoresist pattern may be used, for example, as an etch mask, thereby allowing the pattern to be transferred to one or more sequentially underlying layers by known etching techniques, typically by dry-etching such as reactive ion etching. The photoresist pattern may, for example, be used for pattern transfer to an underlying hardmask layer which, in turn, is used as an etch mask for pattern transfer to one or more layers below the hardmask layer. If the photoresist pattern is not consumed during pattern transfer, it may be removed from the substrate by known techniques, for example, oxygen plasma ashing. The photoresist compositions may, when used in one or more such patterning processes, be used to fabricate semiconductor devices such as memory devices, processor chips (CPUs), graphics chips, optoelectronic chips, LEDs, OLEDs, as well as other electronic devices.

The invention is further illustrated by the following non-limiting examples.

### EXAMPLES

All reactions were carried out under ambient atmospheric conditions. All chemicals were used directly from the supplier. Nuclear magnetic resonance (NMR) spectra for all compounds were obtained on a 500 MHz spectrometer unless otherwise noted. The chemical shifts are reported in 8 (parts per million, ppm) values relative to internal deuterated chloroform residual signal. Multiplicities are indicated by s (singlet), d (doublet), t (triplet), m (multiplet), dd (doublet of doublets), dt (doublet of triplets), tt (triplet of triplets), br (broad singlet).

### Synthesis Examples

### Synthesis of Compound C-i

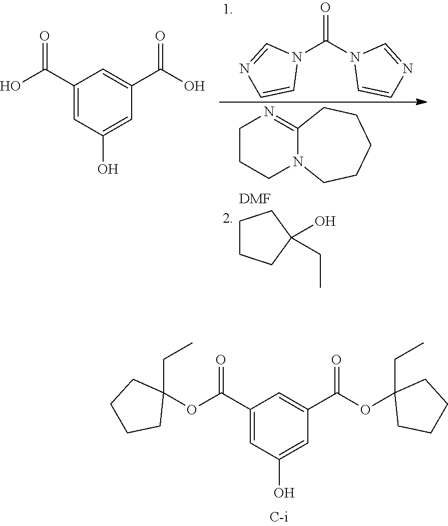

Hydroxyisophthalic acid (30.0 grams (g), 165 millimoles (mmol)) was dissolved in N,N-dimethylformamide (330 milliliters (mL)) to form a solution. Carbonyldiimidazole (57.8 g, 356 mmol) was added portion-wise to the solution to form a reaction mixture. The reaction mixture was heated at 40° C. for 2 hours. Then, 1,8-diazabicyclo(5.4.0) undec-7-ene (54.2 g, 356 mmol) was added to the reaction mixture, followed by the addition of 1-ethylcyclopentanol (56.4 g, 712 mmol). The resulting reaction mixture was heated at 55° C. for 48 hours. The reaction mixture was then cooled to room temperature and water was added thereto (700 mL). The solution was adjusted to a pH of 8 with glacial acetic acid, and then extracted with heptane (700 mL). The organic layer was separated, and then washed with water (3×700 mL) and a saturated sodium bicarbonate aqueous solution (700 mL). The organic layer was subsequently separated, dried over sodium sulfate, filtered, and concentrated under a reduced pressure. The resulting oil was dried under vacuum giving 55.4 g of a vicious clear oil, compound C-i being a ˜74.1 wt % mixture with 1-ethylcyclopentanol (67% yield). ¹H NMR (500 MHz, acetone-d₆) δ 9.06 (br, 1H), 8.08 (s, 1H), 7.65 (s, 2H), 2.30 (m, 4H), 2.14 (q, 4H), 1.83-1.48 (m, 12H), 0.92 (t, 6H).

### Synthesis of Compound C-iii

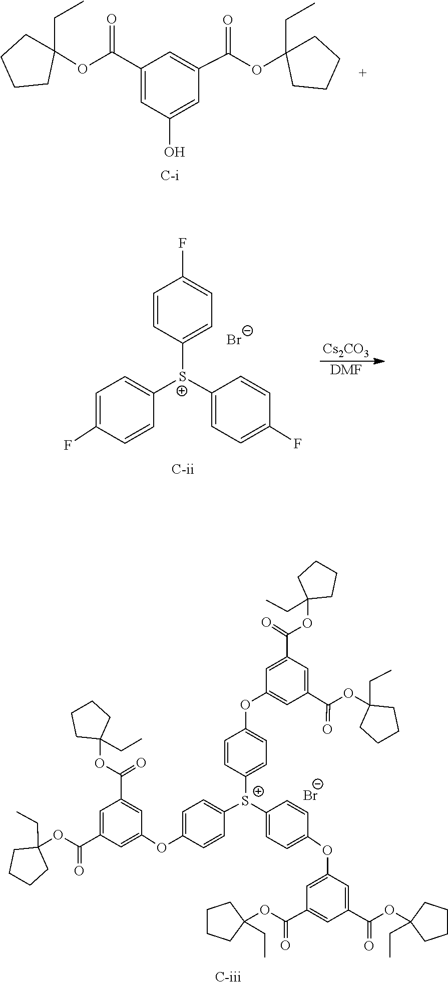

In a 500 mL round bottom flask equipped with a condenser and stirrer, a mixture containing compound C-i (55.4 g, 74.1 wt. %, 110 mmol) and compound C-ii (8.71 g, 21.9 mmol) were dissolved in N,N-dimethylformamide (220 mL) to form a solution. Cesium carbonate (35.7 g, 110 mmol) was added to the solution to form a reaction mixture, and the reaction mixture was heated at 50° C. for about 12 hours. The reaction mixture was then cooled to room temperature and poured into water (400 mL). The solution was extracted with tert-butyl methyl ether (2×300 mL). The combined organic layers were washed with water (2×400 mL), 0.5 M sodium acetate/acetic acid buffer at pH of about 4 (2×400 mL), followed by additional water (2×400 mL). The solution was then dried over filter paper and concentrated under a reduced pressure. The resulting residue was redissolved in acetone (200 mL) and heptane (700 mL) was added slowly thereto. The oil that formed was isolated by decanting the heptane layer and dried under vacuum giving the desired material C-iii as a mixture with C-i and used in next step as a 16.7 wt % solution in dichloromethane. ¹H NMR (500 MHz, acetone-d₆) δ 8.43 (t, 3H), 8.08 (d, 6H), 7.92 (d, 6H), 7.45 (d, 6H), 2.29 (m, 12H), 2.13 (q, 12H), 1.84-1.67 (m, 36H), 0.92 (t, 18H).

### Synthesis of P-1

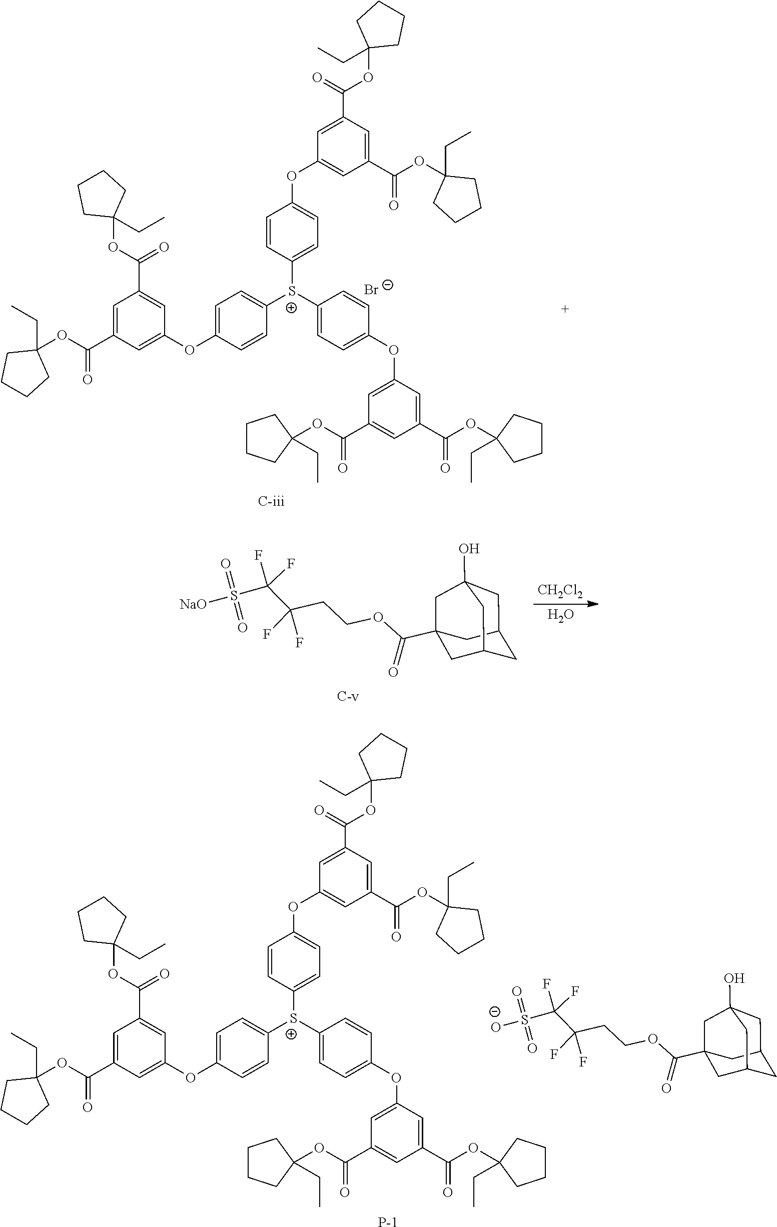

Compound C-iii in dichloromethane (5.01 g, 3.63 mmol) was diluted with dichloromethane (50 mL). Compound C-v (4.64 g, 10.9 mmol) was added followed by water (50 mL). The reaction was stirred for 30 minutes. The layers were separated, and the organic layer was washed with water (50 mL×5). The organic layer was dried over filter paper and concentrated under a reduced pressure. The residue was dissolved in acetone and precipitated using heptane. The solid precipitate was isolated by decanting the solvent and dried under vacuum giving 3.0 g of compound P-1 as a white solid (46%). ¹H NMR (500 MHz, acetone-d₆) δ 8.43 (t, 3H), 8.03 (d, 6H), 7.92 (d, 6H), 7.47 (d, 6H), 4.32 (t, 2H), 2.70 (m, 2H), 2.35-2.10 (m, 26H), 1.89-1.62 (m, 46H), 0.92 (t, 18H).

### Synthesis of P-2

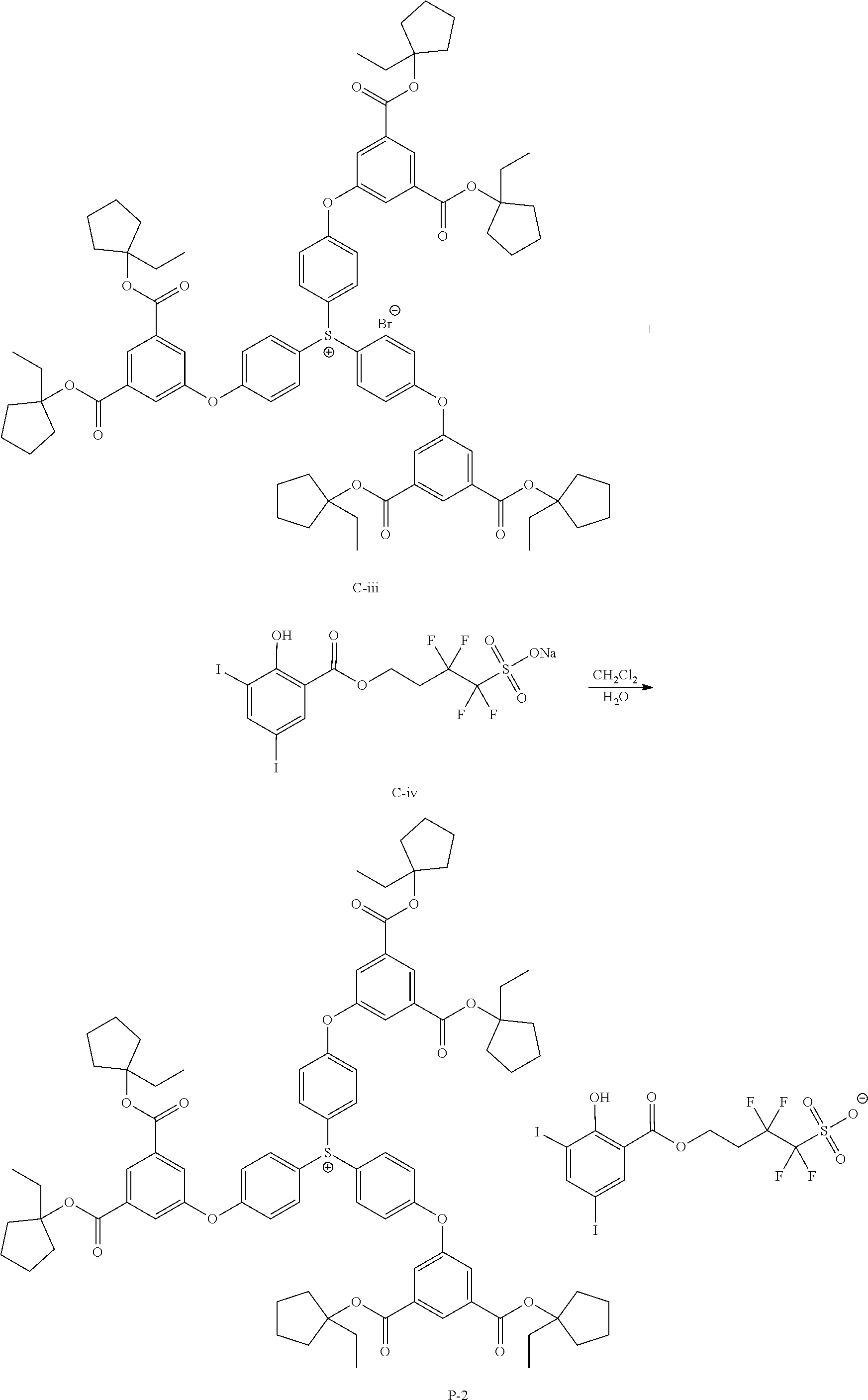

Compound C-iii in dichloromethane (5.01 g, 3.63 mmol) was diluted with dichloromethane (20 mL) to form a solution. Compound C-iv (3.32 g, 5.36 mmol) was then added to the solution, followed by the addition of water (50 mL) to form a reaction mixture. The reaction mixture was stirred for 1 hour. The layers were separated, and the organic layer was washed with water (3×50 mL). The organic layer was then dried over filter paper and concentrated under a reduced pressure. Heptane (200 mL) was added to the resulting residue and heated to 80° C. for 1 hour. The solution was allowed to cool to about 45° C., and the solution was decanted leaving behind an oily material. This residue was dissolved in acetone (20 mL) and heptane (200 mL) was added thereto, and then the resulting mixture was heated to 80° C. for 30 minutes. Then, the solution was decanted. The heptane/acetone precipitation steps were then repeated three more times. The resulting material was dried under vacuum giving 4.00 g of pure P-2 (56% yield). ¹H NMR (500 MHz, acetone-d₆) δ 11.55 (brs, 1H), 8.43 (t, 3H), 8.30 (d, 1H), 8.24 (d, 1H), 8.04 (d, 6H), 7.92 (d, 6H), 7.48 (d, 6H), 4.72 (t, 2H), 2.92 (m, 2H), 2.28 (m, 12H), 2.14 (q, 12H), 1.83-1.68 (m, 36H), 0.92 (t, 18H).

### Synthesis of Compound C-vi

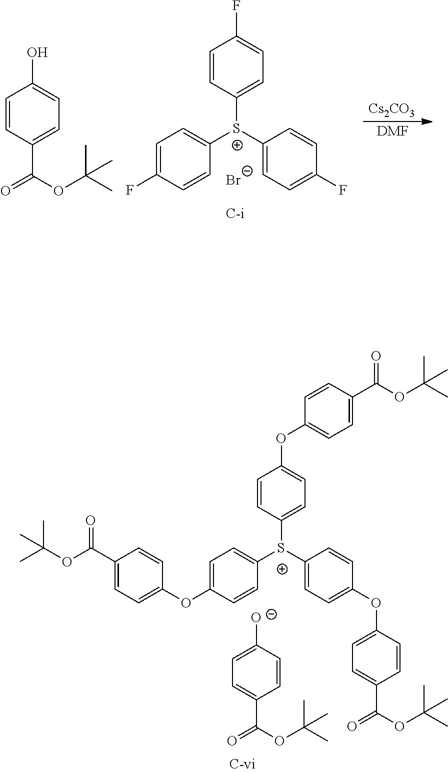

In a 3-neck flask with condenser, thermocouple, and stirrer, compound tert-butyl 4-hydroxybenzoate (19.6 g, 101 mmol) and compound C-i (8.00 g, 20.1 mmol) were dissolved in N,N-dimethylformamide (250 mL). Cesium carbonate (32.8 g, 101 mmol) was added thereto, and the reaction mixture was heated at 55° C. for 2 hours. The reaction mixture was allowed to cool to room temperature before the reaction mixture was partitioned between dichloromethane (350 mL) and water (350 mL). The organic layer was washed with water (3×300 mL) and dried over sodium sulfate. The solution was filtered and concentrated under a reduced pressure. The residue was recrystallized with a mixture of dichloromethane and methyl tert-butyl ether giving 13.8 g of C-vi as a white solid (75% yield). ¹H NMR (500 MHz, acetone-d₆) δ 8.09 (d, 6H), 8.05 (d, 6H), 7.80 (d, 2H), 7.42 (d, 6H), 7.25 (d, 6H), 6.92 (d, 2H), 1.58 (s, 27H), 1.54 (s, 9H).

### Synthesis of P-3

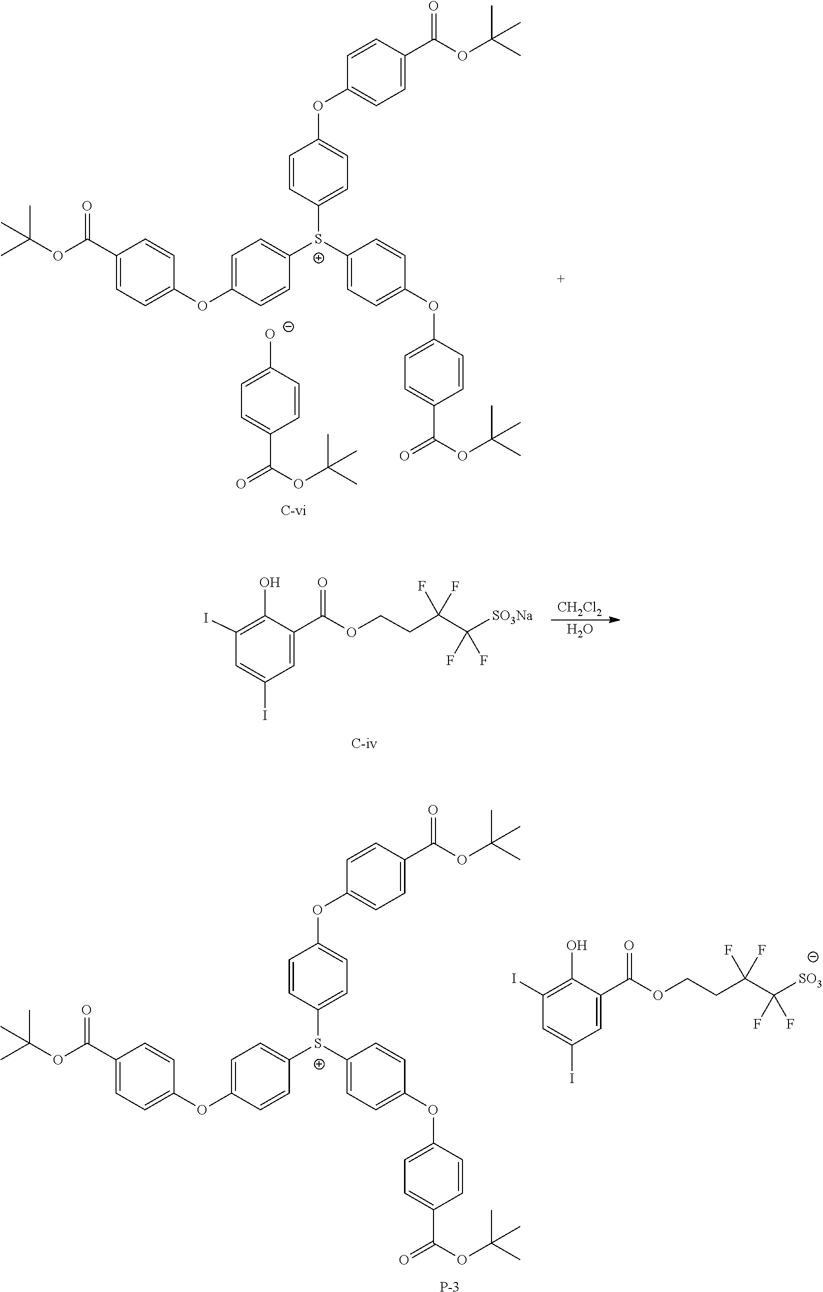

Compound C-vi (13.8 g, 13.3 mmol) and compound C-iv (8.28 g, 13.3 mmol) were partitioned between dichloromethane (100 mL) and water (100 mL), and stirred for 1 hour. The layers were separated, and the organic layer was washed with water (3×100 mL). The organic layer was dried over filter paper and concentrated under a reduced pressure. The residue was dissolved in tert-butyl methyl ether (100 mL), and heptane (600 mL) was added with rapid stirring. The oil that precipitated was isolated by decanting the heptane layer. The residue was redissolved in acetone (100 mL), and heptane (600 mL) was added with rapid stirring. The oil that formed was isolated by decanting the supernatant. The residue was redissolved in MTBE (150 mL) and washed with water (3×100 mL) to remove residual salts. The organic layer was dried over filter paper and concentrated under a reduced pressure. The residue was dissolved in acetone (100 mL), and heptane (600 mL) was added. The oil that precipitated was isolated by decanting the liquid and rinsing with heptane. The product was dried under vacuum to give 14.0 g of P-3 as a white solid (69% yield). ¹H NMR (500 MHz, DMSO-d₆) δ 11.16 (s, 1H), 8.30 (s, 1H), 8.10-7.94 (m, 7H), 7.87 (d, 6H), 7.41 (d, 6H), 7.26 (d, 6H), 4.56 (t, 2H), 2.77 (m, 2H), 1.55 (s, 27H).

### Synthesis of P-4

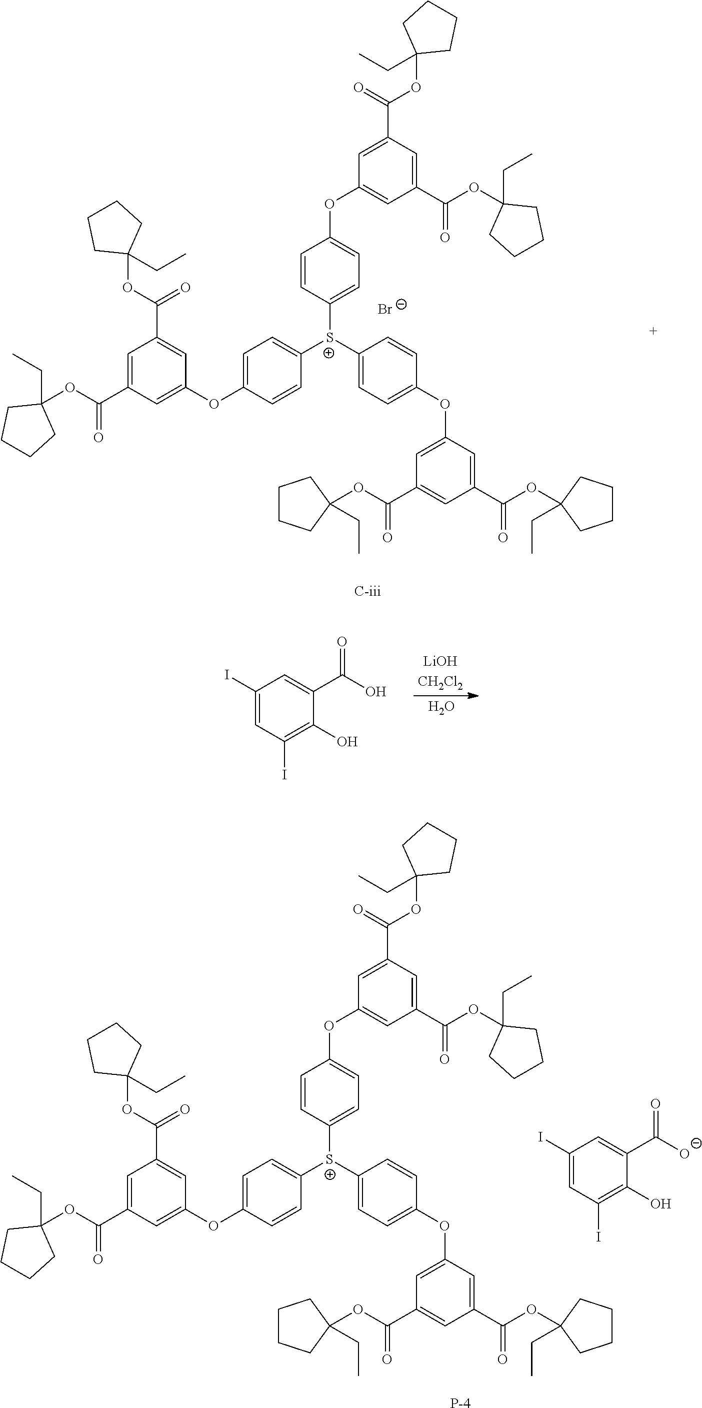

Diiodosalicylic acid (2.12 g, 5.44 mmol) was dissolved in a solution of lithium hydroxide (0.122 g, 5.08 mmol) in water (50 mL) to form a reaction mixture. Compound C-iii (5.01 g, 3.63 mmol) was dissolved in dichloromethane (50 mL) and combined with the reaction mixture. The resulting mixture was stirred for 1 hour. The layers were separated, and the organic layer was washed with saturated sodium bicarbonate solution (50 mL) and water (3×50 mL). The organic layer was then concentrated under a reduced pressure. To the resulting residue, heptane (300 mL) was added, and the resulting mixture was heated to 80° C. for 1 hour. Solids formed, which were filtered while the solution was hot. The solids were redissolved in acetone (80 mL) and heptane (500 mL) was added thereto. The solution was concentrated under a reduced pressure until solid precipitated, and then the solution was heated to 80° C. for 1 hour. The solids were filtered while the solution was hot, and the solids were then rinsed with heptane. The product was dried on filter paper to give 2.52 g of P-4 as a white solid (39%). ¹H NMR (500 MHz, acetone-d₆) δ 8.43 (t, 3H), 8.06 (d, 1H), 8.03 (d, 6H), 7.92 (d, 6H), 7.81 (d, 1H), 7.48 (d, 6H), 2.28 (m, 12H), 2.14 (q, 12H), 1.84-1.67 (m, 36H), 0.92 (t, 18H).

### Synthesis of Compound C-ix

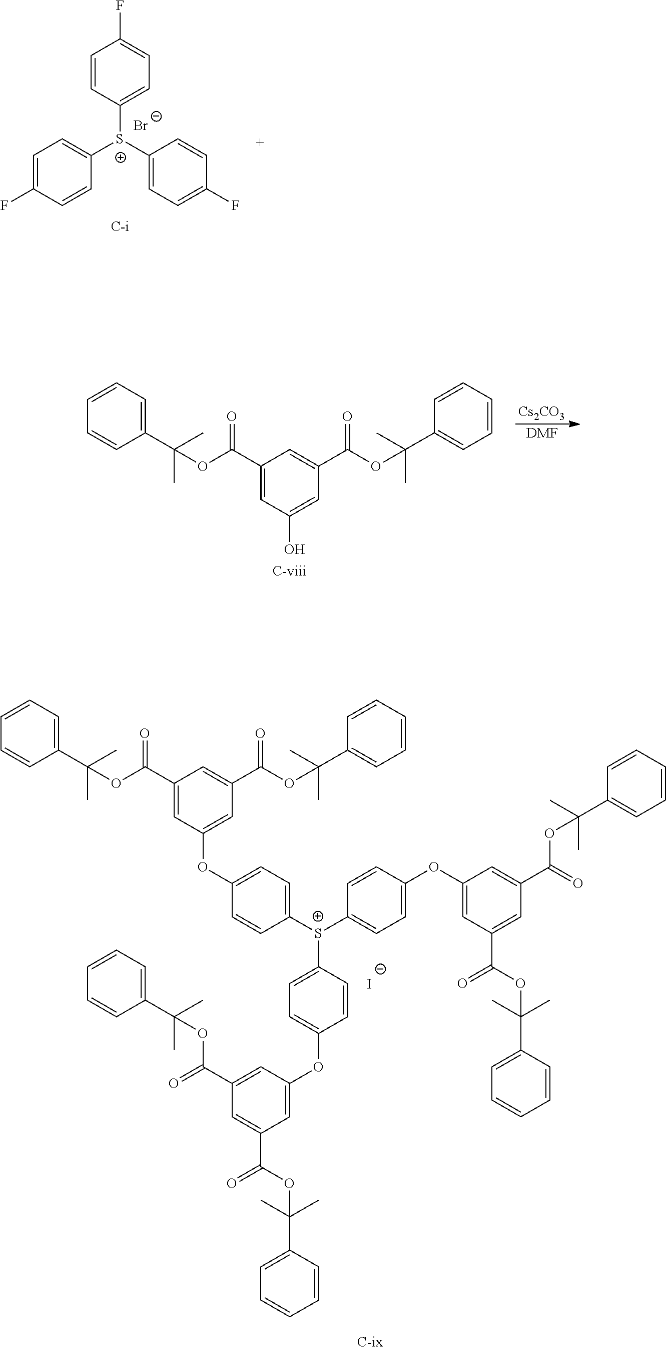

Compound C-i (4.07 g, 10.2 mmol) and compound C-viii (15.0 g, 35.8 mmol) were dissolved in N,N-dimethylformamide (DMF) (100 mL). Cesium carbonate (10.0 g, 30.7 mmol) was added and the reaction was heated at 45° C. for 16 hours. The reaction was cooled to room temperature and was poured into a saturated solution of sodium iodide in water (200 mL). The solid precipitate was filtered and rinsed thoroughly with water. The solid was redissolved in dichloromethane (200 mL) and washed with saturated solution of sodium iodide (3×200 mL) and water (3×200 mL). The organic layer was dried over filter paper and concentrated under a reduced pressure. The resulting residue was triturated with tert-butyl methyl ether (MTBE) (200 mL) and the solvent was discarded. The residue was dissolved in acetone (100 mL) and precipitated out of tert-butyl methyl ether (600 mL). The solid that formed was isolated by decanting the solvent. The precipitation procedure was repeated a second time and the resulting solid was dried under vacuum giving 11.3 g of compound C-ix as a white solid (67%). ¹H NMR (500 MHz, acetone-d₆) δ 8.52 (t, 3H), 8.07 (d, 6H), 7.95 (d, 6H), 7.54-7.45 (m, 12H), 7.42 (d, 6H), 7.34 (t, 12H), 7.26 (d, 6H), 1.92 (s, 36H).

### Synthesis of P-5

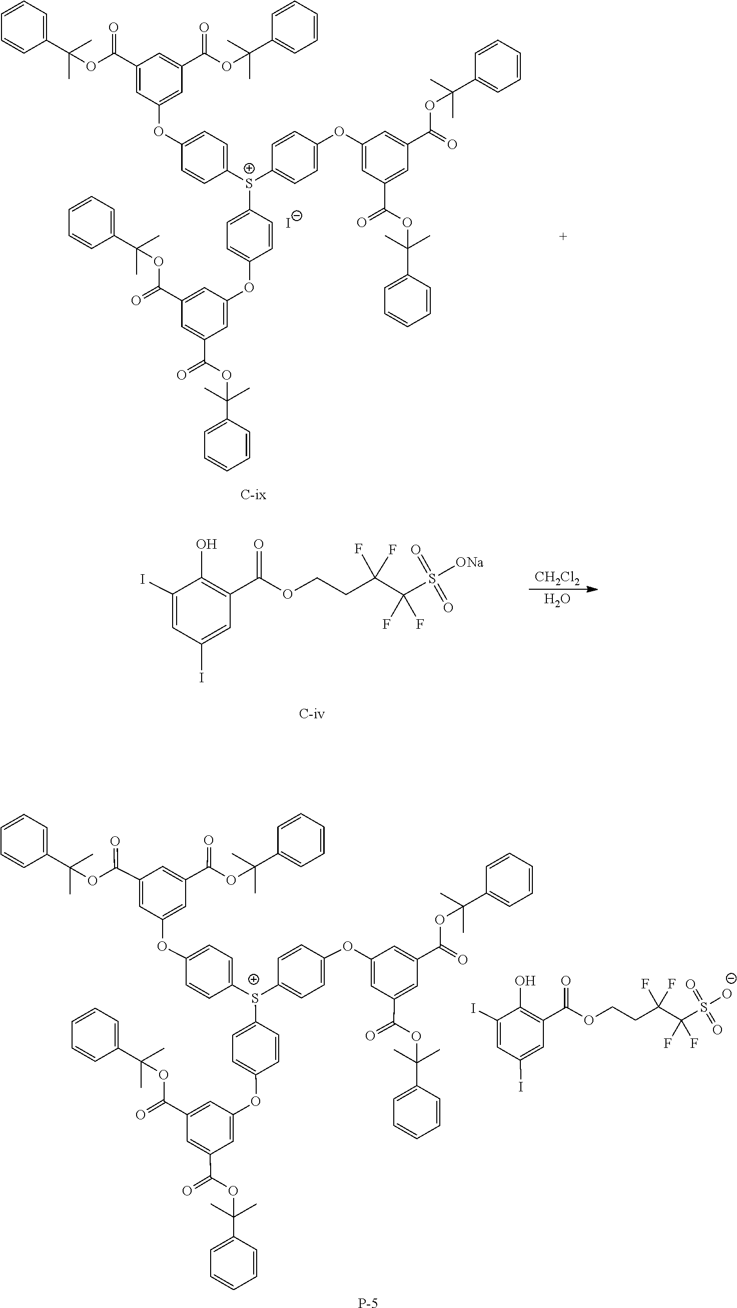

Compound C-ix (11.3 g, 6.89 mmol) and compound C-iv (4.27 g, 6.89 mmol) were partitioned between dichloromethane (100 mL) and water (100 mL). The reaction mixture was then stirred for 30 minutes at room temperature. The layers were separated and the organic layer was washed with water (3×100 mL). The organic layer was dried over filter paper and concentrated under a reduced pressure. The resulting residue was dissolved in a mixture of acetone (20 mL) and tert-butyl methyl ether (100 mL), and then the product was precipitated using heptane (700 mL). The solid that precipitated was isolated by decanting the supernatant and dried under vacuum giving 9.5 g of compound P-5 as a white solid (66%). ¹H NMR (500 MHz, acetone-d₆) δ 11.52 (s, 1H), 8.52 (s, 3H), 8.27 (d, 1H), 8.21 (d, 1H), 7.99 (d, 6H), 7.94 (s, 6H), 7.48 (d, 12H), 7.43 (d, 6H), 7.34 (t, 12H), 7.26 (t, 6H), 4.69 (t, 2H), 2.90 (m, 2H), 1.92 (s, 36H).

### Synthesis of C-x

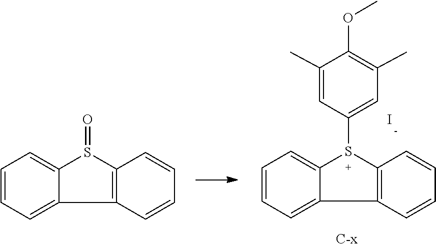

2,6-dimethylanisole (22.4 g, 165 mmol) followed by Eaton's Reagent (60 mL) were added sequentially in one portion to a solution of dibenzothiophene oxide (30.0 g, 150 mmol) in dichloromethane (60 mL) at room temperature, and the reaction mixture was stirred overnight. The reaction was quenched by the slow addition of water (250 mL) and stirred under a reduced pressure until the appearance of a precipitate formed. The precipitate was filtered and the mother liquor was washed with MTBE (2×200 mL). Any subsequent precipitation was further filtered. Next, sodium iodide (300 mmol) in water (200 mL) was added to the aqueous phase under vigorous stirring. The resulting precipitate was filtered, washed with copious amounts of water, and suspended in a minimal amount of acetone and stirred at 35° C. for 2 hours. The solution was then cooled to room temperature and filtered to afford compound C-x (46.1 g, 69%) as a white solid. ¹H NMR (300 MHz, DMSO-d₆) δ: 8.52 (d, J=8.1 Hz, 2H), 8.33 (d, J=7.8 Hz, 2H), 7.96 (t, J=7.5 Hz, 2H), 7.75 (t, J=7.5 Hz, 2H), 7.32 (s, 2H), 3.68 (s, 3H), 2.19 (s, 6H).

### Synthesis of C-xi

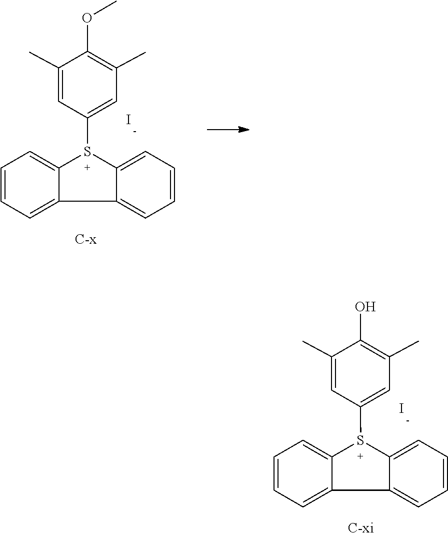

Boron tribromide (10.2 mL, 108 mmol) was added dropwise to a solution of the iodide salt C-x (32.0 g, 71.7 mmol) in dichloromethane (300 mL) at room temperature, and the reaction mixture as stirred for 2 hours. The reaction mixture was cooled to 0° C. and quenched by the slow addition of water (50 mL). The resulting precipitate was filtered, washed with acetone (100 mL), and dried to afford compound C-xi (24.7 g, 80%) as a white solid. ¹H NMR (300 MHz, DMSO-d₆) δ: 9.77 (brs, 1 OH), 8.52 (d, J=8.1 Hz, 2H), 8.27 (8.1 Hz, 2H), 7.95 (t, J=7.8 Hz, 2H), 7.74 (t, J=7.8 Hz, 2H), 7.20 (s, 2H), 2.12 (s, 6H), −1.39 (m, 5H), 1.11 (s, 3H), 0.83-0.89 (m, 6H).

### Synthesis of C-ix

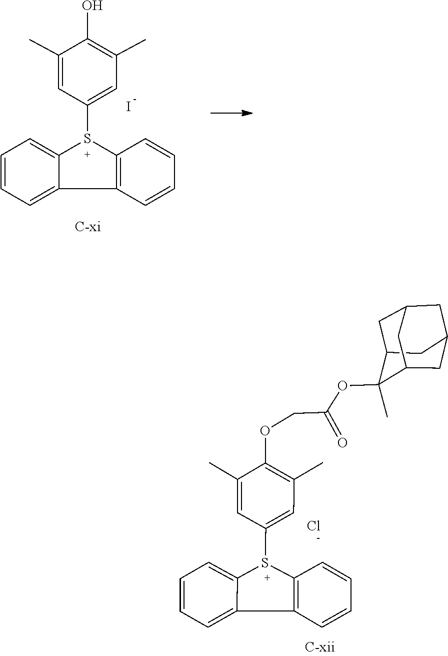

Compound C-xi (24.4 g, 56.4 mmol), cesium carbonate (25.7 g, 79.0 mmol), sodium iodide (16.9 g, 113 mmol), and (1R,3S,5r,7r)-2-methyladamantan-2-yl 2-chloroacetate (14.4 g, 59.2 mmol) were dissolved in DMF (250 mL) and stirred at room temperature overnight. The reaction mixture was poured onto water (400 mL) and extracted with dichloromethane (3×150 mL). Thee combined organic layers were washed with water (4×300 mL) and concentrated under a reduced pressure to a viscous oil which was precipitated into MTBE (1.5 L) and filtered to afford compound C-xii (34.5 g, 96%) as a white solid.

### Synthesis of P-6

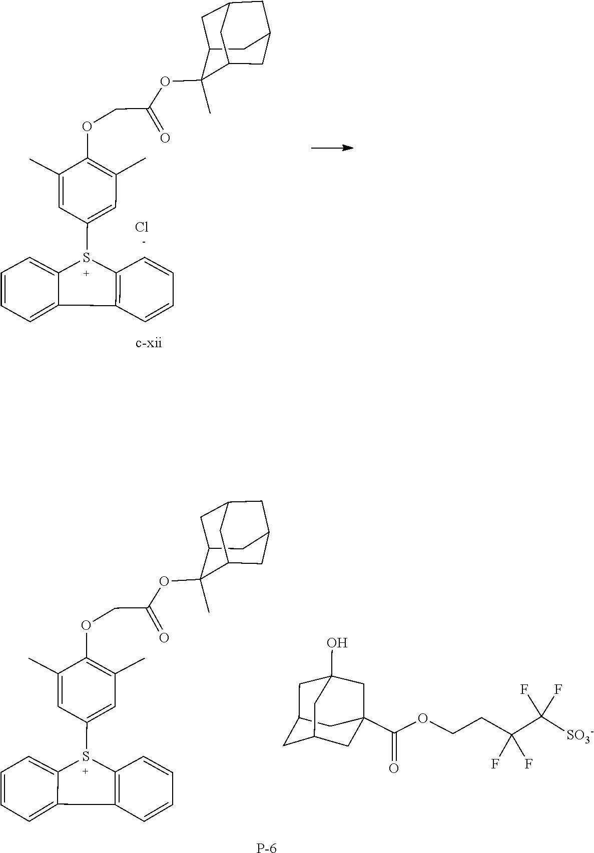

Compound C-xii (66.0 g, 0.121 mmol) and sodium 1,1,2,2-tetrafluoro-4-(((1r,3s,5R,7S)-3-hydroxyadamantane-1-carbonyl)oxy) butane-1-sulfonate (51.4 g, 0.121 mmol) were dissolved in dichloromethane (1 L) and water (1 L) and stirred at room temperature overnight. The layers were separated, the organic layer washed with water (10×500 mL), and the combined organic layers were concentrated under a reduced pressure to afford compound P-6 (92.2 g, 84%) as an off white solid. ¹H NMR (300 MHz, DMSO-d₆) δ: 8.51 (d, J=7.8 Hz, 2H), 8.32 (d, J=8.1 Hz, 2H), 7.96 (t, J=7.8 Hz, 2H), 7.76 (t, J=7.8 Hz, 2H), 7.31 (s, 2H), 4.55 (s, 2H), 4.23 (t, J=6.6 Hz, 2H), 3.40 (s, 1OH), 2.40-2.60 (m, 2H), 1.35-2.27 (m, 37H).

### Synthesis of P-7

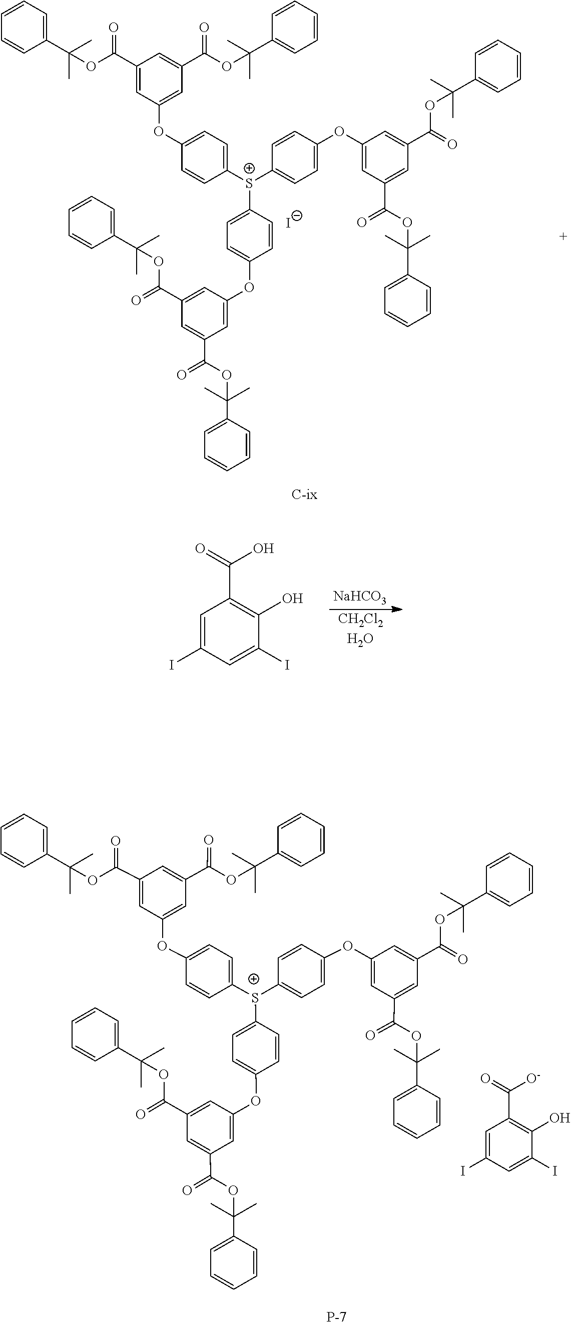

Compound C-ix (7.00 g, 4.27 mmol) and 3,5-diiodosalicylic acid (2.50 g, 6.40 mmol) were partitioned between DCM (70 mL) and a saturated solution of sodium bicarbonate in water (70 mL). The reaction was stirred for 1 hour. The layers were separated and the organic layer was washed with saturated solution of sodium bicarbonate in water (70 mL) and water (70 mL×5). The organic layer was passed over filter paper and concentrated under reduced pressure. The residue was purified by precipitating from an acetone/heptane mixture and dried under vacuum giving 5.66 g of compound P-7 (70%). ¹H NMR (500 MHz, DMSO-d₆) δ 8.36 (t, 3H), 7.93-7.81 (m, 13H), 7.81-7.77 (m, 1H), 7.47-7.16 (m, 36H), 1.83 (s, 36H).

### Synthesis of C-xiv

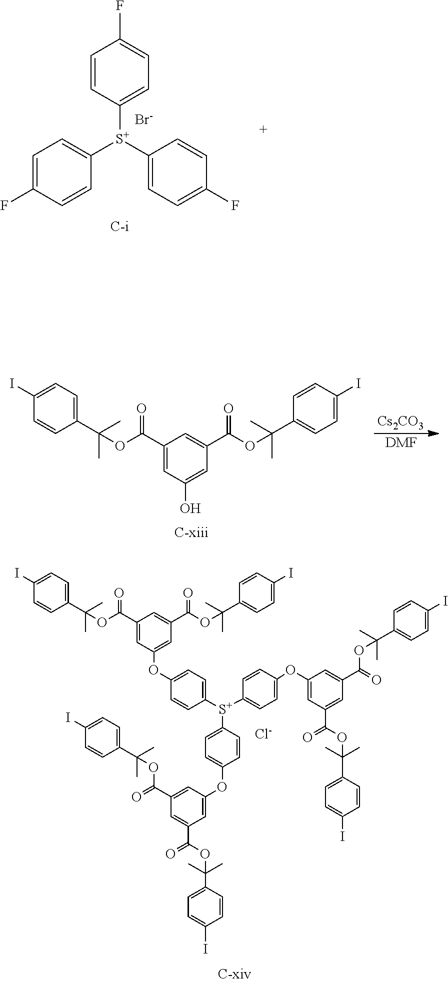

Compound C-i (7.65 g, 19.3 mmol) and compound C-xiii (40.0 g, 59.7 mmol) were dissolved in DMF (160 mL). Cesium carbonate (18.8 g, 57.8 mmol) was added and the reaction was heated at 45° C. for 48 hours. The reaction was then cooled to room temperature and was poured into a saturated solution of sodium chloride in water (800 mL). The solid precipitate was filtered and rinsed thoroughly with brine followed by water. The solid was redissolved in ethyl acetate (500 mL) and passed over filter paper. The solution was precipitated with MTBE, filtered, and dried under vacuum to give 32.4 g of compound C-xiv (77%). ¹H NMR (500 MHz, DMSO-d₆) δ 8.33 (t, 3H), 7.96-7.78 (m, 12H), 7.78-7.61 (m, 13H), 7.45-7.33 (m, 6H), 7.27-7.12 (m, 13H), 1.80 (s, 38H).

### Synthesis of Compound P-8

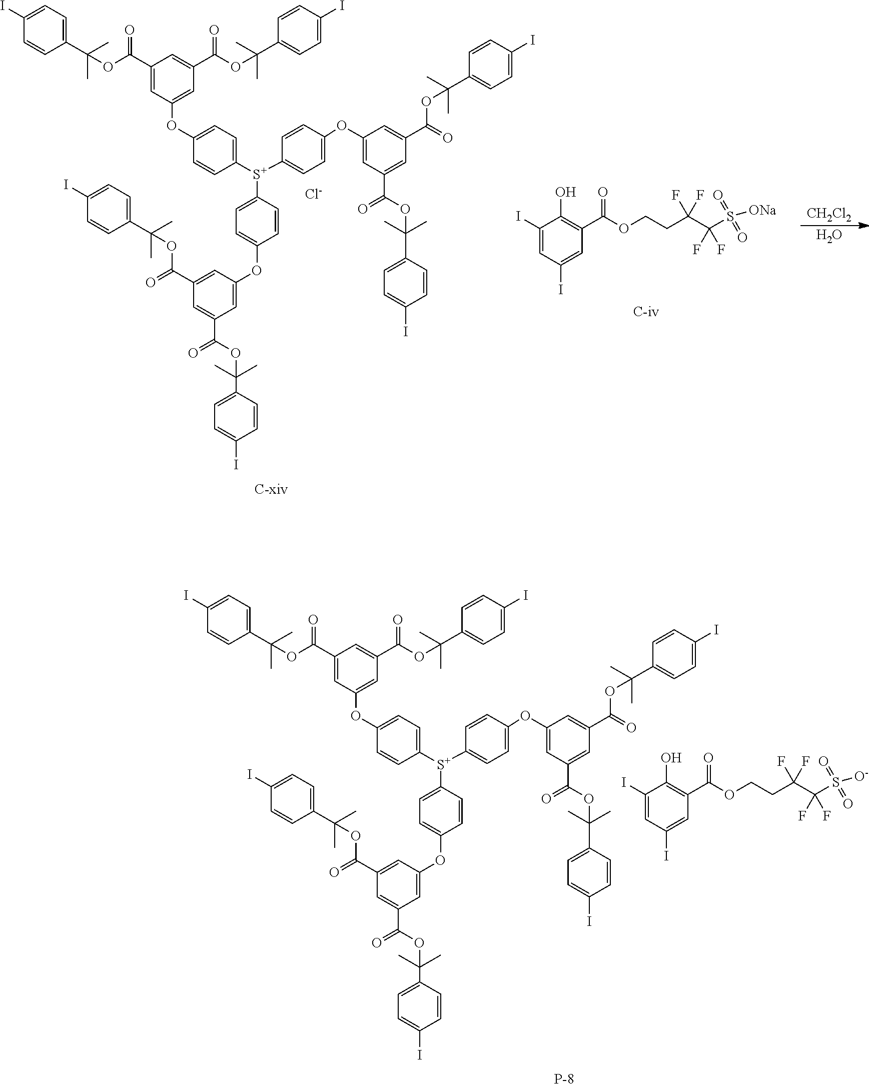

Compound C-xiv (10.0 g, 4.34 mmol) and compound C-iv (2.75 g, 4.43 mmol) were partitioned between water (100 mL) and dichloromethane (100 mL). The mixture was then stirred rapidly for 16 hours. The layers were separated and the organic layer was washed with water (100 mL×5). The organic layer was passed over filter paper and concentrated under reduced pressure. The residue was precipitated from a mixture of acetone, MTBE, and heptane. The resulting solid was filtered and dried under vacuum to give 8.06 g of compound P-8-(65%). ¹H NMR (500 MHz, DMSO-d₆) δ 11.15 (s, 1H), 8.33 (t, 3H), 8.26 (d, 1H), 8.00 (d, 1H), 7.92-7.80 (m, 12H), 7.67 (d, 12H), 7.39 (d, 6H), 7.22 (d, 12H), 4.55 (s, 2H), 2.75 (s, 2H), 1.80 (s, 37H).

### Synthesis of Compound P-9

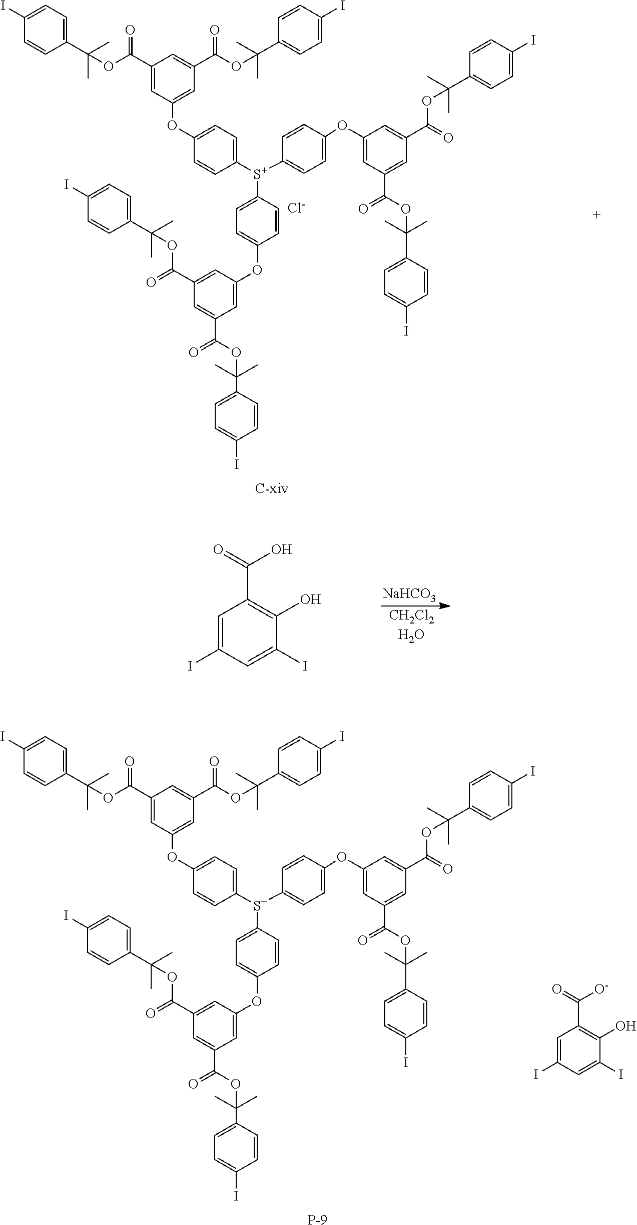

Compound C-xiv (10.0 g, 4.34 mmol) and 3,5-diiodosalicylic acid (2.03 g, 5.21 mmol) were partitioned between DCM (100 mL) and a saturated solution of sodium bicarbonate in water (100 mL). The reaction was stirred for 1 hour. The layers were separated and the organic layer was washed with a saturated solution of sodium bicarbonate in water (100 mL×2) and water (100 mL×5). The organic layer was passed over filter paper and concentrated under reduced pressure. The residue was purified by precipitating from an acetone/MTBE mixture and dried under vacuum giving 7.40 g of compound P-9 (64%). ¹H NMR (500 MHz, DMSO-d₆) δ 8.33 (t, 3H), 7.97-7.76 (m, 14H), 7.67 (d, 12H), 7.39 (d, 6H), 7.22 (d, 12H), 1.80 (s, 36H).

### Synthesis of Compound P-10

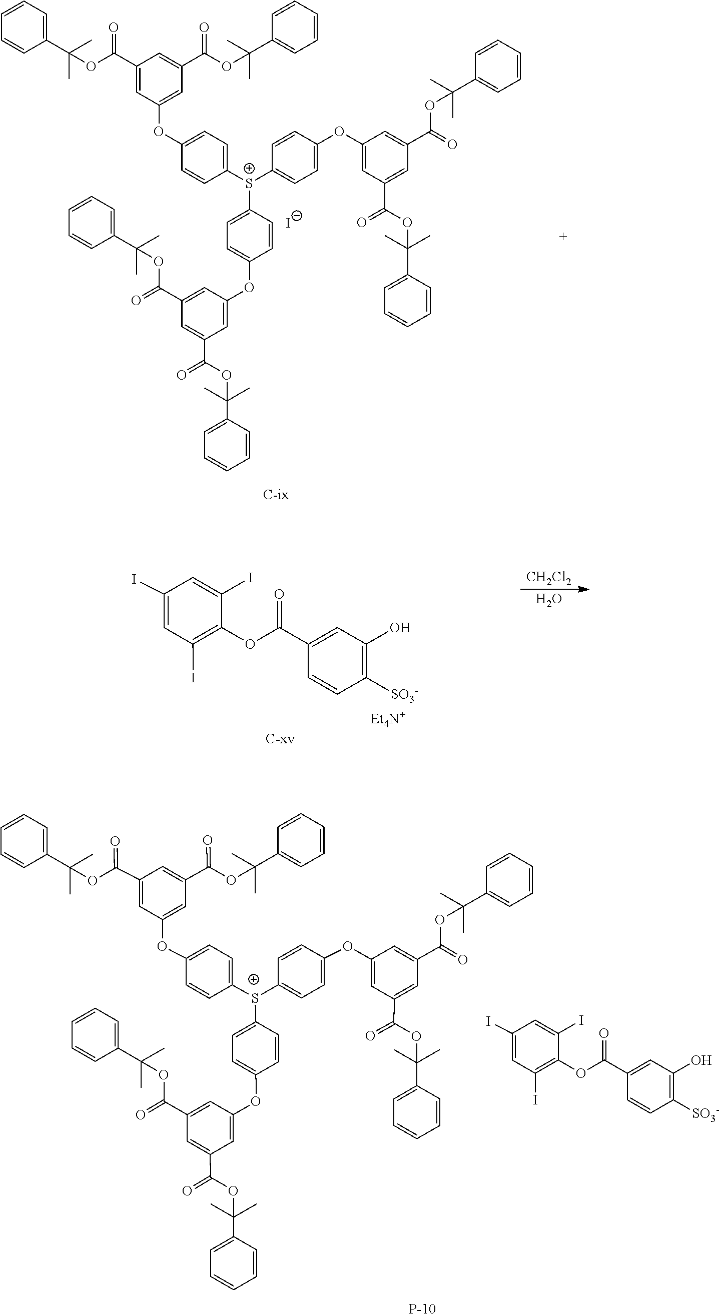

Compound C-ix (6.93 g, 4.23 mmol) and compound C-xv (3.40 g, 4.25 mmol) were partitioned between dichloromethane (50 mL) and water (50 mL). The layers were separated and the organic layer was washed with water (50 mL×4) and passed over filter paper. The solution was added to heptane (600 mL). The resulting solid was filtered and dried under vacuum to give 6.89 g of compound P-10 (75%). ¹H NMR (500 MHz, DMSO-d₆) δ 10.76 (d, 1H), 8.36 (t, 3H), 8.23 (d, 2H), 7.91-7.76 (m, 12H), 7.70 (m, 1H), 7.60 (m, 1H), 7.50 (d, 1H), 7.48-7.35 (m, 18H), 7.35-7.28 (m, 12H), 7.24 (m, 7H), 1.83 (s, 36H).

### Photoresist Compositions and Evaluation

Photoresist compositions were prepared by dissolving solid components in solvents using the materials and amounts indicated in Table 1, where the amounts are expressed in wt % based on 100 wt % of total solids. The total solids content for the photoresist compositions was 2.2 wt %. The solvent system contained PGMEA (50 wt %) and methyl-2-hydroxyisobutyrate (50 wt %). Each mixture was shaken using a mechanical shaker and then filtered through a PTFE disk-shaped filter having a pore size of 0.2 micron.

Photolithography was performed using a CLEAN TRAC ACT8 (TEL, Tokyo Electron Co.) wafer track. 200 nm wafers for photolithographic testing were coated with an AR™ 3 BARC (DuPont Electronic Materials International) and softbaked at 205° C. for 60 seconds to give a 60 nm film. A photoresist composition was then coated onto the AR™ 3 BARC stack and soft-baked at 110° C. for 60 seconds to give a photoresist film layer having a thickness of about 60 nm.

The wafers were exposed with 248 nm radiation on a CANON FPA-5000 ES4 scanner (NA=0.8, outer sigma=0.85, inner sigma=0.57) with a mask having the features of choice. The wafers were post-exposure baked at 90° C. for 60 seconds, developed with MF™ CD26 TMAH developer (DuPont Electronic Materials International) for 60 seconds, rinsed with DI water, and dried. Critical dimension (CD) linewidth measurements of the formed patterns were made using a HITACHI S-9380 CD-SEM. Sizing energy (E_{size}) of the lines were determined based on the 180 nm CD size. P/Q=mole of component one/mole of quencher.

TABLE 1

| Photoresist | Component | Component | | | E_{size} |
|---|---|---|---|---|---|
| Example | One | Two | Quencher | P/Q | (mJ/cm²) |
| CPR-1* | P-3 | — | Q1 | 8.67 | 51.13 |
| | [97.47] | | [2.53] | | |
| PR-1 | P-3 | A1 | Q1 | 8.67 | 48.14 |
| | [88.80] | [8.88] | [2.32] | | |
| CPR-2* | P-2 | — | Q1 | 8.62 | 114.59 |
| | [98.14] | | [1.86] | | |
| PR-2 | P-2 | A1 | Q1 | 8.62 | 96.45 |
| | [89.4] | [8.94] | [1.66] | | |
| CPR-3* | P-2 | — | P-4 | 8.62 | 138.10 |
| | [90.60] | | [9.4] | | |
| PR-3 | P-2 | A1 | P-4 | 8.62 | 117.69 |
| | [83.08] | [8.31] | [8.61] | | |
| CPR-4* | P-1 | — | Q1 | 8.65 | 72.36 |
| | [97.94] | | [2.06] | | |
| PR-4 | P-1 | A1 | Q1 | 8.65 | 69.75 |
| | [89.21] | [8.92] | [1.87] | | |
| CPR-5* | P-5 | — | Q1 | 8.63 | 84.23 |
| | [98.25] | | 1.75 | | |
| PR-5 | P-5 | A1 | Q1 | 8.63 | 57.11 |
| | [89.46] | [8.95] | [1.59] | | |
| PR-6 | P-5 | A1 | Q1 | 8.63 | 39.93 |
| | [70.53] | [28.21] | [1.26] | | |
| PR-7 | P-5 | A2 | Q1 | 8.63 | 77.52 |
| | [89.46] | [8.95] | [1.59] | | |
| PR-8 | P-5 | A3 | Q1 | 8.63 | 46.6 |
| | [89.46] | [8.95] | [1.59] | | |
| PR-9 | P-5 | A4 | Q1 | 8.63 | 66.53 |
| | [93.65] | [4.68] | [1.67] | | |
| CPR-6* | P-6 | — | Q1 | 18.66 | No |
| | [98.14] | | [1.86] | | printing |
| PR-10 | P-6 | A1 | Q1 | 18.66 | 39.57 |
| | [70.47] | [28.39] | [1.14] | | |

*denotes a comparative example.

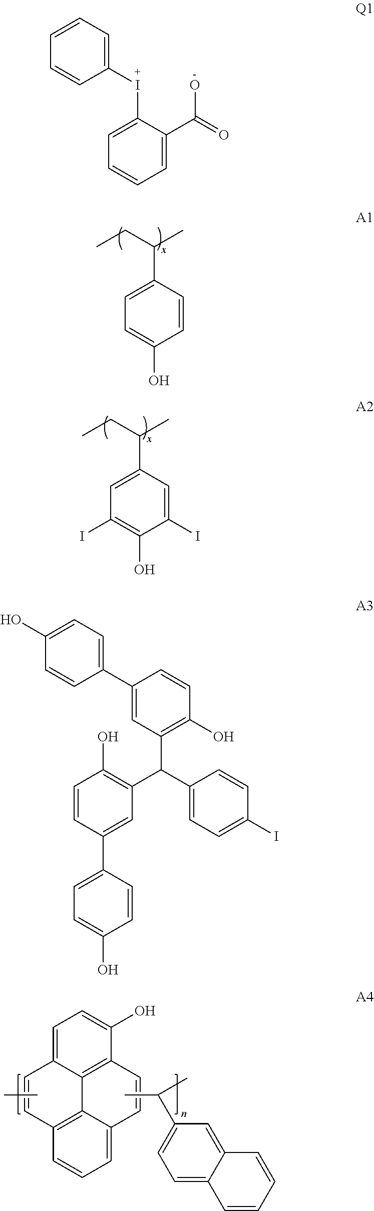

As can be seen in Table 1, the photoresist compositions PR-1 to PR-9 achieved faster photospeed (a decreased E_{size} value) relative to comparative photoresist composition CPR-1 to CPR-5 respectively in L/S patterning under KrF exposure. In addition, the photoresist composition PR-10 achieved lithographic patterning, whereas CPR-6 did not provide a printed pattern.

### EUV Evaluation (I)

Photoresist compositions were prepared by dissolving solid components in solvents using the materials and amounts indicated in Table 2, where the amounts are expressed in wt % based on 100 wt % of total weight of the solids. The total solids content for the photoresist compositions was 2.2 wt %. The solvent system contained propylene glycol monomethyl ether acetate (PGMEA) (50 wt %) and methyl-2-hydroxyisobutyrate (HBM) (50 wt %). Each mixture was shaken using a mechanical shaker and then filtered through a PTFE disk-shaped filter having a pore size of 0.2 micron.

EUV exposure was conducted on 300 mm wafers, wherein photoresist compositions were spin coated onto a 60 nm organic BARC, 20 nm SiARC stack to achieve a 45 nm film thickness, followed by a post application bake at 110° C. for 60 seconds. EUV exposure was conducted on an ASML NXE3400B (NA=0.33) scanner using a hole pattern mask having a hole CD of 20 nm at a 40 nm pitch. The exposed wafers were post-exposure baked at 100° C. for 60 seconds, developed with a 0.26 N TMAH solution for 60 seconds, rinsed with DI water, and spun dry to reveal the hole pattern. Hole CD measurements were made using a Hitachi CG5000 CD-SEM. Dose-to-size (E_{size}) was determined from exposure latitude plots of dose vs. CD, which was the exposure dose that provided a hole pattern having a size of 20 nm and is reported in millijoules per square centimeter (mJ/cm²). The results are reported in Table 2.

TABLE 2

| Photoresist | Component | Component | | E_{size} |
|---|---|---|---|---|
| Example | One | Two | Quencher | (mJ/cm² |
| CPR-7* | P-5 | | Q-1 | 70 |
| | [97.75] | | [2.25] | |
| PR-11 | P-5 | A1 | Q-1 | 46.4 |
| | [89.05] | [8.9] | [2.05] | |
| PR-12 | P-10 | A1 | P-7 | 54.1 |
| | [86.78] | [8.68] | [4.54] | |
| PR-13 | P-5 | A3 | Q-1 | 57.5 |
| | [88.68] | [8.87] | [2.45] | |
| PR-14 | P-5 | A1 | P-7 | 38.8 |
| | [83.02] | [8.31] | [8.68] | |

As can be seen in Table 2, the photoresist compositions PR-11 to PR-14 achieved faster photospeed relative to comparative photoresist composition CPR-7 respectively in contact hole patterning under EUV exposure.

### EUV Evaluation (II)

Photoresist compositions were prepared by dissolving solid components in solvents using the materials and amounts indicated in Table 2, where the amounts are expressed in wt % based on 100 wt % of total weight of the solids. The total solids content for the photoresist compositions was 2.2 wt %. The solvent system contained PGMEA (50 wt %) and propylene glycol monomethyl ether (PGME) (50 wt %). Each mixture was shaken using a mechanical shaker and then filtered through a PTFE disk-shaped filter having a pore size of 0.2 micron.

EUV exposure was conducted on 300 mm wafers, wherein photoresist compositions were spin coated onto a 60 nm organic BARC, 20 nm SiARC stack to achieve a 45 nm film thickness, followed by a post application bake at 110° C. for 60 seconds. EUV exposure was conducted on an ASML NXE3400B (NA=0.33) scanner using a hole pattern mask having a hole CD of 24 nm at a 45 nm pitch. The exposed wafers were post-exposure baked at 100° C. for 60 seconds, developed with a 0.26 N TMAH solution for 60 seconds, rinsed with DI water, and spun dry to reveal the hole pattern. Hole CD measurements were made using a Hitachi CG5000 CD-SEM. Dose-to-size (E_{size}) was determined from exposure latitude plots of dose vs. CD, which was the exposure dose that provided a hole pattern having a size of 24 nm and is reported in millijoules per square centimeter (mJ/cm²). The results are reported in Table 3.

TABLE 3

| Photoresist | | | | E_{size} |
|---|---|---|---|---|
| Example | Compound 1 | Compound 2 | Quencher | (mJ/cm²) |
| CPR-8* | P-8 | | P-9 | 52.85 |
| | [87.8] | | [12.2] | |
| PR15 | P-8 | A1 | P-9 | 42.45 |
| | [76.7] | [7.7] | [15.6] | |

As can be seen in Table 3, the photoresist compositions PR-15 achieved faster photospeed relative to comparative photoresist composition CPR-8 respectively in contact hole patterning under EUV exposure.

While this disclosure has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1. A photoresist composition, comprising:
a first component;
a second component; and
a solvent,
wherein the first component comprises a non-polymeric onium salt acid generator, and a cation of the non-polymeric onium salt generator comprises an acid labile group,
wherein the second component comprises a hydroxyaryl group, and the second component does not comprise an acid labile group, and
wherein the photoresist composition does not comprise a polymer that comprises an acid labile group.

2. The photoresist composition of claim 1, wherein the first component is a salt comprising a cation of Formula (1) or (2): 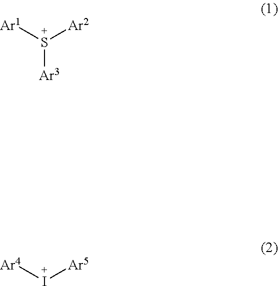
wherein, in Formulae (1) and (2),
Ar¹ to Ar⁵ are each independently substituted or unsubstituted C₆₋₃₀ aryl, or substituted or unsubstituted C₃₋₃₀ heteroaryl,
wherein each of Ar¹ to Ar³ may be either separate or connected to another group Ar¹ to Ar³ via a single bond or a divalent linking group to form a ring,
wherein each of Ar⁴ to Ar⁵ may be either separate or connected to another group Ar⁴ to Ar⁵ via a single bond or a divalent linking group to form a ring,
wherein at least one of Ar¹ to Ar³ in Formula (1) and at least one of Ar⁴ to Ar⁵ in Formula (2) comprises an acid labile group.

3. The photoresist composition of claim 1, wherein the first component further comprises an anion selected from halide, hexafluorophosphate, or an anion group, wherein the anion group comprises sulfonate, sulfonamidate, sulfonimidate, methide, borate, or carboxylate.

4. The photoresist composition of claim 1, wherein the second component is a polymer comprising a repeating unit of Formula (3): 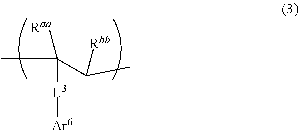
wherein, in Formula (3),
R^{aa} is hydrogen, halogen, cyano, substituted or unsubstituted C₁₋₁₀ alkyl, or substituted or unsubstituted C₁₋₁₀ fluoroalkyl,
R^{bb} is hydrogen, —C(O)— forming a ring with L³, or a single bond forming a ring with Ar⁶,
L³ is a single bond or a divalent linking group comprising one or more of substituted or unsubstituted C₁₋₃₀ alkylene, substituted or unsubstituted C₃₋₃₀ cycloalkylene, substituted or unsubstituted C₁₋₃₀ heterocycloalkylene, substituted or unsubstituted C₆₋₃₀ arylene, substituted or unsubstituted C₇₋₃₀ arylalkylene, substituted or unsubstituted C₁₋₃₀ heteroarylene, or substituted or unsubstituted C₃₋₃₀ heteroarylalkylene, —O—, —C(O)—, —C(O)—O—, —C(O)—N(R^{2a}), —S—, —S(O)₂—, —N(R^{2a})—S(O)₂—, —C₁₋₁₂ hydrocarbylene-, —O—(C₁₋₁₂ hydrocarbylene)-, —C(O)—O—(C₁₋₁₂ hydrocarbylene)-, or —C(O)—O—(C₁₋₁₂ hydrocarbylene)-O—, wherein R^{2a} is hydrogen, C₁₋₆ alkyl, or a single bond forming the ring with R^{bb},
provided that R^{bb} is —C(O)— forming the ring with L³ when R^{2a} is the single bond forming the ring with R^{bb}, and
Ar⁶ comprises a C₆₋₃₀ aryl group, a C₃₋₃₀ heteroaryl group, or a combination thereof, each comprising at least one hydroxy substituent, and each optionally further substituted with one or more non-hydrogen substituents.

5. The photoresist composition of claim 1, wherein the second component is a compound of Formula (4): 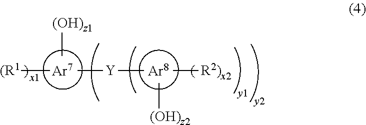
wherein, in Formula (4),
Ar⁷ and each Ar⁸ are independently C₆-30 arylene, or C₃₋₃₀ heteroarylene,
R¹ and R² are each independently a non-hydrogen substituent,
each Y is independently a single bond, or a multivalent linking group, wherein the multivalent linking group is substituted or unsubstituted,
x1 and x2 are each independently an integer from 0 to 10,
y1 is an integer from 1 to 5,
y2 is an integer from 0 to 10, and
z1 and z2 are each independently an integer from 0 to 10, provided z1+z2 is at least 2.

6. The photoresist composition of claim 1, wherein the second component is a condensation polymer derived from the monomer of Formula (5): 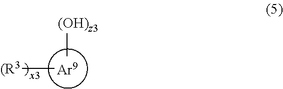
wherein, in Formula (5),
Ar⁸ is C₆₋₃₀ arylene or C₃₋₃₀ heteroarylene,
R³ is a non-hydrogen substituent,
x3 is an integer from 0 to 5, and
z3 is an integer from 1 to 10.

7. The photoresist composition of claim 1, wherein the second component comprises two or more hydroxyaryl groups.

8. The photoresist composition of claim 1, wherein the first component is present in the photoresist composition in an amount of 50 weight percent or greater, based on total solids of the photoresist composition.

9. The photoresist composition of claim 1, further comprising a photo-decomposable quencher or a basic quencher that is different from the second component.

10. The photoresist composition of claim 1, further comprising a photoacid generator.

11. A method for forming a pattern, the method comprising:
applying a layer of a photoresist composition of claim 1 on a substrate to provide a photoresist composition layer;
pattern-wise exposing the photoresist composition layer to activating radiation to provide an exposed photoresist composition layer; and
developing the exposed photoresist composition layer to provide the pattern.

12. The method of claim 11, wherein the first component further comprises an anion selected from halide, hexafluorophosphate, or an anion group, wherein the anion group comprises sulfonate, sulfonamidate, sulfonimidate, methide, borate, or carboxylate.

13. The method of claim 11, wherein the second component is a polymer comprising a repeating unit of Formula (3): 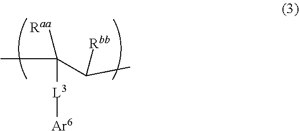
wherein, in Formula (3),
R^{aa} is hydrogen, halogen, cyano, substituted or unsubstituted C₁₋₁₀ alkyl, or substituted or unsubstituted C₁₋₁₀ fluoroalkyl,
R^{bb} is hydrogen, —C(O)— forming a ring with L³, or a single bond forming a ring with Ar⁶,
L³ is a single bond or a divalent linking group comprising one or more of substituted or unsubstituted C₁₋₃₀ alkylene, substituted or unsubstituted C₃₋₃₀ cycloalkylene, substituted or unsubstituted C₁₋₃₀ heterocycloalkylene, substituted or unsubstituted C₆₋₃₀ arylene, substituted or unsubstituted C₇₋₃₀ arylalkylene, substituted or unsubstituted C₁₋₃₀ heteroarylene, or substituted or unsubstituted C₃₋₃₀ heteroarylalkylene, —O—, —C(O)—, —C(O)—O—, —C(O)—N(R^{2a}), —S—, —S(O)₂—, —N(R^{2a})—S(O)₂—, —C₁₋₁₂ hydrocarbylene-, —O—(C₁₋₁₂ hydrocarbylene)-, —C(O)—O—(C₁₋₁₂ hydrocarbylene)-, or —C(O)—O—(C₁₋₁₂ hydrocarbylene)-O—, wherein R^{2a} is hydrogen, C₁₋₆ alkyl, or a single bond forming the ring with R^{bb};
provided that R^{bb} is —C(O)— forming the ring with L³ when R^{2a} is the single bond forming the ring with R^{bb}, and
Ar⁶ comprises a C₆₋₃₀ aryl group, a C₃₋₃₀ heteroaryl group, or a combination thereof, each comprising at least one hydroxy substituent, and each optionally further substituted with one or more non-hydrogen substituents.

14. The method of claim 11, wherein the second component is a compound of Formula (4): 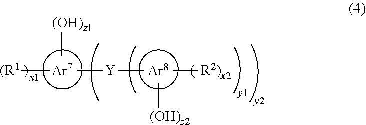
wherein, in Formula (4),
Ar⁷ and each Ar⁸ are independently C₆-30 arylene, or C₃₋₃₀ heteroarylene,
R¹ and R² are each independently a non-hydrogen substituent,
each Y is independently a single bond, or a multivalent linking group, wherein the multivalent linking group is substituted or unsubstituted,
x1 and x2 are each independently an integer from 0 to 10,
y1 is an integer from 1 to 5,
y2 is an integer from 0 to 10, and
z1 and z2 are each independently an integer from 0 to 10, provided z1+z2 is at least 2.

15. The method of claim 11, wherein the second component is a condensation polymer derived from the monomer of Formula (5): 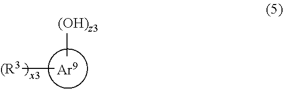
wherein, in Formula (5),
Ar⁸ is C₆₋₃₀ arylene or C₃₋₃₀ heteroarylene,
R³ is a non-hydrogen substituent,
x3 is an integer from 0 to 5, and
z3 is an integer from 1 to 10.

16. The method of claim 11, wherein the second component comprises two or more hydroxyaryl groups.

17. The method of claim 11, wherein the first component is present in the photoresist composition in an amount of 50 weight percent or greater, based on total solids of the photoresist composition.

18. The method of claim 11, further comprising a photo-decomposable quencher or a basic quencher that is different from the second component in the photoresist composition.

19. The method of claim 11, further comprising a photoacid generator in the photoresist composition.
